(19) United States
(12) Patent Application Publication
(10) Pub. No.: US 2005/0148631 A1
(43) Pub. Date: Jul. 7, 2005

(54) Amino heterocyclyl inhibitors of 11-beta-hydroxy steroid dehydrogenase type 1

(75) Inventors: Hengmiao Cheng, San Diego, CA (US); Stephan James Cripps, San Diego, CA (US); Martin Paul Edwards, San Diego, CA (US); Theodore Otto Johnson JR., San Diego, CA (US); Sajiv Krishnan Nair, Vista, CA (US); Michael Siu, San Diego, CA (US); Christopher Ronald Smith, San Diego, CA (US); Wendy Dianne Taylor, San Diego, CA (US); Yong Wang, San Diego, CA (US)

Correspondence Address: AGOURON PHARMACEUTICALS, INC., 10350 NORTH TORREY PINES ROAD, LA JOLLA, CA, 92037, US

(73) Assignee: AGOURON PHARMACEUTICALS, INC.

(21) Appl. No.: 11/016,152

(22) Filed: Dec. 17, 2004

Related U.S. Application Data
(60) Provisional application No. 60/531,186, filed on Dec. 19, 2003.
(60) Provisional application No. 60/556,921, filed on Mar. 26, 2004.

(51) Int. Cl. A61K 31/4545; A61K 31/4436; A61K 31/44; C07D 49/02; C07D 41/02; C07D 213/02

(52) U.S. Cl. 514/318; 514/339; 514/352; 546/194; 546/281.4; 546/304

(57) ABSTRACT

The present invention relates to compounds with the formula (I), or a pharmaceutically acceptable salt thereof: 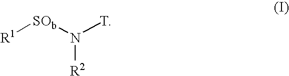 The invention also relates to pharmaceutical compositions comprising the compounds of formula (I) or formula (II) and methods of treating a condition that is mediated by the modulation of 11-β-hsd-1, the method comprising administering to a mammal an effective amount of a compound of formula (I) or formula (II).

This application claims the benefit of U.S. Application Ser. No. 60/531,186 filed Dec. 19, 2003 and U.S. Application Ser. No. 60/556,921 filed Mar. 26, 2004, hereby incorporated by reference in its entirety for all purposes.

### FIELD OF THE INVENTION

The present invention relates to novel compounds, to pharmaceutical compositions comprising the compounds, as well as to the use of the compounds in medicine and for the preparation of a medicament which acts on the human 11-β-hydroxysteroid dehydrogenase type 1 enzyme (11-β-hsd-1).

### BACKGROUND OF THE INVENTION

It has been known for more than half a century that glucocorticoids have a central role in diabetes. For example, the removal of the pituitary or the adrenal gland from a diabetic animal alleviates the most severe symptoms of diabetes and lowers the concentration of glucose in the blood (Long, C. D. and F. D. W. Leukins (1936) J. Exp. Med. 63: 465-490; Houssay, B. A. (1942) Endocrinology 30: 884-892). Additionally, it is also well established that glucocorticoids enable the effect of glucagon on the liver.

The role of 11-β-hsd-1 as an important regulator of local glucocorticoid effects and thus of hepatic glucose production is well substantiated (see e.g. Jamieson et al. (2000) J. Endocrinol. 165: p. 685-692). The hepatic insulin sensitivity was improved in healthy human volunteers treated with the non-specific 11-β-hsd-1 inhibitor carbenoxolone (Walker, B. R., et al. (1995) J. Clin. Endocrinol. Metab. 80: 3155-3159). Furthermore, the expected mechanism has been established by different experiments with mice and rats. These studies showed that the mRNA levels and activities of two key enzymes in hepatic glucose production were reduced, namely the rate-limiting enzyme in gluconeogenesis, phosphoenolpyruvate carboxykinase (PEPCK), and glucose-6-phosphatase (G6Pase) catalyzing the last common step of gluconeogenesis and glycogenolysis. Finally, the blood glucose level and hepatic glucose production was reduced in mice having the 11-β-hsd-1 gene knocked-out. Data from this model also confirms that inhibition of 11-β-hsd-1 will not cause hypoglycemia, as predicted, since the basal levels of PEPCK and G6Pase are regulated independently of glucocorticoids (Kotelevtsev, Y., et al., (1997) Proc. Natl. Acad. Sci. USA 94: 14924-14929).

Abdominal obesity is closely associated with glucose intolerance, hyperinsulinemia, hypertriglyceridemia, and other factors of the so-called Metabolic Syndrome (e.g. raised blood pressure, decreased levels of HDL and increased levels of VLDL) (Montague & O'Rahilly, Diabetes 49: 883-888, 2000). Obesity is an important factor in Metabolic Syndrome as well as in the majority (>80%) of type 2 diabetic, and omental fat appears to be of central importance. Inhibition of the enzyme in pre-adipocytes (stromal cells) has been shown to decrease the rate of differentiation into adipocytes. This is predicted to result in diminished expansion (possibly reduction) of the omental fat depot, i.e. reduced central obesity (Bujalska, I. J., Kumar, S., and Stewart, P. M. (1997) Lancet 349: 1210-1213).

The discussion of the background to the invention herein is included to explain the context of the invention. This is not to be taken as an admission that any of the material referred to was published, known or part of the common general knowledge in the United States as at the priority date of any of the claims.

The compounds of the present invention are 11 β-hsd-1 inhibitors, and are therefore believed to be useful in the treatment of diabetes, obesity, glaucoma, osteoporosis, cognitive disorders, immune disorders, depression, hypertension, and metabolic diseases.

### SUMMARY OF THE INVENTION

The present invention relates to a compound of formula (I): 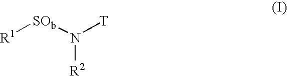
wherein:
R¹ is selected from the group consisting of (C₁-C₆)alkyl, —(CR³R⁴)ₜ(C₃-C₁₂)cycloalkyl, —(CR³R⁴)ₜ(C₆-C₁₂)aryl, and —(CR³R⁴)ₜ(4-10)-membered heterocyclyl wherein said —(CR³R⁴)ₜ(4-10)-membered heterocyclyl is optionally substituted on a nitrogen atom by a substituent selected from the group consisting of —CF₃, —CHF₂, —CH₂F, (C₁-C₆)alkyl, (C₂-C₆)alkenyl, (C₂-C₆)alkynyl, —(C═O)—R³, —(C═O)—O—R³, —(CR³R⁴)ₜ(C₆-C₁₂ aryl), —(CR³R⁴)ₜ(4-10)-membered heterocyclyl, —(CR³R⁴)ₜ—(C═O)(CR³R⁴)ᵤ(C₆-C₁₂)aryl, and —(CR³R⁴)ₜ—(C═O)(CR³R⁴)ᵤ(4-10)- membered heterocyclyl;
b and k are each independently selected from 1 and 2;
j is selected from the group consisting of 0, 1, and 2;
t, u, p, q, and v are each independently selected from the group consisting of 0, 1, 2, 3, 4, and 5;
T is a (6-10)-membered heterocyclyl containing at least one nitrogen atom;
R² is selected from the group consisting of H, (C₁-C₆)alkyl, —(CR³R⁴)ₜ(C₃-C₁₂)cycloalkyl, —(CR³R⁴)ₜ(C₆-C₁₂)aryl, and —(CR³R⁴)ₜ(4-10)-membered heterocyclyl;
each R³ and R⁴ is independently selected from H and (C₁-C₆)alkyl;
the carbon atoms of T, R¹, R², R³ and R⁴ may each be optionally substituted by 1 to 5 R⁵ groups;
each R⁵ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(C═O)—R⁶, —(C═O)—O—R⁶, —R⁶—O—R⁷, —R⁶—(C═O)—R⁷, —R⁶—(C═O)—OR⁷, —O—R⁶, —O—(C═O)—R⁷, —O—(C═O)—NR⁷R⁸, —NR⁸—(C═O)—R⁹, —R⁷—(C═O)—NR⁸R⁹, —(C═O)—NR⁸R⁹, —R⁶—(C═O)—NR⁸R⁹, —NR⁸R⁹, —NR⁸OR⁹, —S(O)ₖNR⁸R⁹, —S(O)ⱼ(C₁-C₆)alkyl, —O—S(O)ₖ—R⁹, —NR⁸—S(O)ₖ—R⁹, —(CR⁸R⁹)ᵥ(C₃-C₁₂)cycloalkyl, —(CR¹⁰R¹¹)ᵥ(C₆-C₁₂ aryl), —(CR¹⁰R¹¹)ᵥ(4-10)-membered heterocyclyl, —(CR¹⁰R¹¹)_{q}—(C═O)(CR¹⁰R¹¹)ᵥ(C₆-C₁₂)aryl —(CR¹⁰OR¹¹)_{q}—(C═O)(CR¹⁰R¹¹)ᵥ(4-10)-membered heterocyclyl, —(CR¹⁰R¹¹)O(CR¹⁰OR¹¹)_{q}(C₆-C₁₂)aryl, —(CR¹⁰R¹¹)ᵥO(CR¹⁰R¹¹)_{q}(4-10)-membered heterocyclyl, —(CR¹⁰R¹¹)_{q}S(O)ⱼ (CR¹⁰OR¹¹)ᵥ(C₆-C₁₂)aryl, and —(CR¹⁰R¹¹)_{q}S(O)ⱼ (CR¹⁰OR¹¹)ᵥ(4-10)-membered heterocyclyl;
any 1 or 2 carbon atoms of any (4-10)-membered heterocyclyl of the foregoing R⁵ groups are optionally substituted with an oxo (═O);
any nitrogen atom of any (4-10)-membered heterocyclyl of the foregoing R⁵ group is optionally substituted with (C₁-C₆)alkyl or (CR¹⁰OR¹¹)ᵥ(C₆-C₁₂)aryl;
any carbon atom of any (C₁-C₆)alkyl, any (C₆-C₁₂)aryl, and any (4-10)-membered heterocyclyl of the foregoing R⁵ groups are optionally substituted with 1 to 3 substituents independently selected from halo, hydroxy, cyano, nitro, —CF₃, —CFH₂, —CF₂H, trifluoromethoxy, azido, —OR¹², —R¹²—O—R¹³, —(C═O)—R¹², —(C═O)—O—R¹³, —O—(C═O)—R¹³, —NR¹³—(C═O)—R¹⁴, —(C═O)—NR¹⁵R¹⁶, —NR¹⁷R¹⁸, —SR¹⁴, —S(O)ⱼ(C₁-C₆)alkyl, —NR¹⁴OR¹⁵, (C₁-C₆)alkyl, (C₂-C₆)alkenyl, (C₂-C₆)alkynyl, —(CR¹⁶R¹⁷)ᵤ(C₆-C₁₂)aryl, and —(CR¹⁶R¹⁷)ᵤ(4-10)-membered heterocyclyl;
each R⁶, R⁷, R⁸, R⁹, R¹⁰, R¹¹, R¹², R¹³, R¹⁴, R¹⁵, R¹⁶ and R¹⁷ group is independently selected from the group consisting of H, (C₁-C₆)alkyl, —(C═O)N(C₁-C₆)alkyl, —(CR¹⁸R¹⁹)ₚ(C₆-C₁₂)aryl, —(CR¹⁸R¹⁹)ₚ(C₃-C₁₂)cycloalkyl, and —(CR¹⁸R¹⁹)ₚ(4-10)-membered heterocyclyl;
any 1 or 2 carbon atoms of the (4-10)-membered heterocyclyl of each said R⁶, R⁷, R⁸, R⁹, R¹⁰, R¹¹, R¹², R¹³, R¹⁴, R¹⁵, R¹⁶, R¹⁷ group is optionally substituted with an oxo (═O);
any carbon atom of any (C₁-C₆)alkyl, any (C₆-C₁₂)aryl, and any (4-10)-membered heterocyclyl of the foregoing R⁶, R⁷, R⁸, R⁹, R¹⁰, R¹¹, R¹², R¹³, R¹⁴, R¹⁵, R¹⁶, R¹⁷ groups are optionally substituted with 1 to 3 substituents independently selected from the group consisting of halo, cyano, nitro, —NR²⁰R²¹, —CF₃, —CHF₂, —CH₂F, trifluoromethoxy, (C₁-C₆)alkyl, (C₂-C₆)alkenyl, (C₂-C₆)alkynyl, hydroxy, and (C₁-C₆) alkoxy;
each R¹⁸, R¹⁹, R²⁰, and R²¹ group is independently selected from H and (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from the group consisting of hydroxy, halo, (C₁-C₆)alkyl, (C₁-C₆)alkoxy, —NH₂, —NH(C₁-C₆)(alkyl) and —N((C₁-C₆)(alkyl))₂;
or a pharmaceutically acceptable salt or solvate thereof.

In another embodiment, the invention relates to a compound according to formula (I), wherein b is 2.

In yet another embodiment, the invention relates to a compound according to formula (I), wherein T is a 6-membered heterocyclyl containing at least one nitrogen atom.

In an embodiment, the invention relates to a compound according to formula (I), wherein said T a (6-10)-membered heterocyclyl selected from the group consisting of

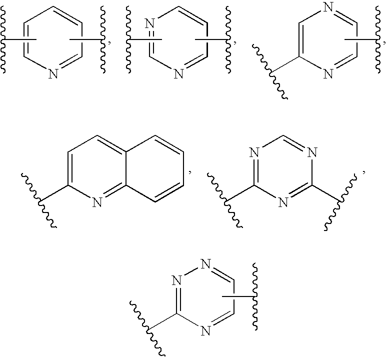

In yet another embodiment, the invention relates to a compound according to formula (I) wherein T is

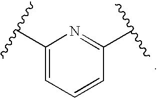

In yet another embodiment, the invention relates to a compound according to formula (I), wherein T is

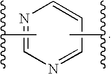

In an embodiment, the invention relates to a compound according to formula (I), wherein T is

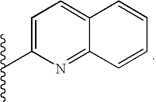

In another embodiment, the invention relates to a compound according to formula (I), wherein each R¹ is selected from the group consisting of phenyl, benzothiophenyl, thiazolyl, pyridinyl, piperazinyl, and napthyl and may optionally be substituted by 1 to 5 R⁵ groups;

wherein:

each R⁵ group is independently selected from the group consisting of halo, cyano, —CF₃, hydroxy, (C₁-C₆)alkoxy, (C₁-C₆)alkyl, (C₂-C₆)alkenyl, —(CR¹⁰OR¹¹)ᵥ(4-10)-membered heterocyclyl, —(C═O)—R⁶, —(C═O)—13 R⁶, —O—(C═O)—R⁷, —NR⁸—(C═O)—R⁹, —(C═O)—NR⁸R⁹, —NR⁸R⁹, —NR⁸OR⁹, —(CR¹⁰OR¹¹)ᵥ—O—(CR¹⁰R¹¹)ₚ(C₆-C₁₂)aryl, and —(CR¹⁰R¹¹)ₚ—O—(CR¹⁰OR¹¹)ₚ(4-10)-membered heterocyclyl.

The invention relates to a compound according to formula (II): 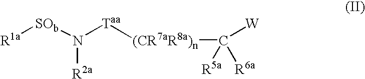
wherein:
R^{1a} is (C₁-C₆)alkyl, —(CR^{7a}R^{8a})ₜ(C₃-C₁₀)cycloalkyl, —(CR^{7a}R^{8a})ₜ(C₆-C₁₀)aryl, or —(CR^{7a}R^{8a})ₜ(4-10)-membered heterocyclyl;
b and k are each independently selected from 1 and 2;
n and j are each independently selected from the group consisting of 0, 1, and 2;
t, u, p, q and v are each independently selected from the group consisting of 0, 1, 2, 3, 4, and 5;
T^{aa} is a (6-10)-membered heterocyclyl containing at least one nitrogen atom;
W is selected from the group consisting of: 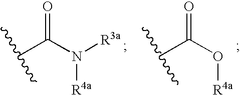
   (C₁-C₆) alkyl; and a 5-membered heterocyclyl;
each R^{2a}, R^{3a}, and R^{4a} is independently selected from the group consisting of H, (C₁-C₆)alkyl, (C₂-C₆)alkenyl, (C₂-C₆)alkynyl, —(CR^{7a}R^{2a})ₜ(C₃-C₁₀)cycloalkyl, —(CR^{7a}R^{8a})ₜ(C₆-C₁₀)aryl, and —(CR^{7a}R^{8a})ₜ(4-10)-membered heterocyclyl;
or each R^{3a} and R^{4a} may optionally be taken together with the nitrogen to which they are attached to form a (4-10)-membered heterocyclyl, and the carbon atoms of the (4-10)-membered heterocyclyl may be optionally substituted by 1 to 5 R^{9a} groups;
or each R^{3a} and R^{4a} may optionally be taken together with the nitrogen to which they are attached to form a (4-10)-membered heterocyclyl containing at least one nitrogen atom wherein said at least one nitrogen atom is optionally substituted by at least one substituent selected from the group consisting of —CF₃, —CHF₂, —CH₂F, trifluoromethoxy, (C₁-C₆)alkoxy, (C₁-C₆)alkyl, (C₂-C₆)alkenyl, (C₂-C₆)alkynyl, —(C═O)—R^{7a}, —(C═O)—O—R^{7a}, —(CR^{7a}R^{8a})ᵥ(C₆-C₁₂)aryl, —(CR^{7a}R^{8a})ᵥ(4-10)-membered heterocyclyl, —(CR^{7a}R^{8a})_{q}—(C═O)(CR^{7a}R^{8a})ᵥ(C₆-C₁₂)aryl, and —(CR^{7a}R^{8a})_{q}—(C═O)(CR^{7a}R^{8a})ᵥ(4-10)-membered heterocyclyl;
each R^{5a} and R^{8a} are independently selected from the group consisting of H, (C₁-C₆) alkyl, —(CR^{7a}R^{8a})ₜ(C₃-C₁₀)cycloalkyl, —(CR^{7a}R^{8a})ₜ(C₆-C₁₀)aryl, and —(CR^{7a}R^{8a})ₜ(4-10)-membered heterocyclyl;
or R^{5a} and R^{6a} may optionally be taken together with the carbon to which they are attached to form a (C₃-C₆)cycloalkyl or a (3-7)-membered heterocyclyl;
each R^{7a} and R^{8a} is independently selected from H and (C₁-C₆)alkyl;
the carbon atoms of T^{aa}, R^{1a}, R^{2a}, R^{3a}, R^{4a}, R^{5a}, R^{6a}, R^{7a}, R^{8a}, and said W 5-membered heterocyclyl are optionally substituted by 1 to 5 R^{9a} groups;
each R^{9a} group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(C═O)—R^{10a}, —(C═O)—O—R^{11a}, —O—(C═O)—R^{11a}, —NR^{11a}—(C═O)—R^{12a}, —(C═O)—NR^{11a}R^{12a}, —NR^{11a}R^{12a}, —NR^{11a}OR^{12a}, —S(O)ₖNR^{11a}R^{12a}, —S(O)ⱼ(C₁-C₆)alkyl, —O—SI₂—R^{10a}, —NR^{11a}—S(O)ₖ—R^{12a}, —(CR^{13a}R^{14a})ᵥ(C₆—C₁₀)aryl, —(CR^{13a}R^{14a})ᵥ(4-10)-membered heterocyclyl, —(CR^{13a}R^{14a})_{q}—(C═O)(CR^{13a}R^{14a})ᵥ(C₆-C₁₀)aryl, —(CR^{13a}R^{14a})_{q}—(C═O)(CR^{13a}R^{14a})ᵥ(4-10-membered heterocyclyl, —(CR^{13a}R^{14a})ᵥO(CR^{13a}R^{14a})_{q}(C₆-C₁₀)aryl, —(CR^{13a}R^{14a})ᵥO(CR^{13a}R^{14a})_{q}(4-10)-membered heterocyclyl, —(CR^{13a}R^{14a})_{q}S(O)ⱼ(CR^{13a}R^{14a})ᵥ(C₆-C₁₀)aryl, and —(CR^{13a}R^{14a})_{q}S(O)ⱼ(CR^{13a}R^{14a})ᵥ(4-10)-membered heterocyclyl;
any 1 or 2 carbon atoms of any (4-10)-membered heterocyclyl of the foregoing R^{9a} groups are optionally substituted with an oxo (═O);
any carbon atom of any (C₁-C₆)alkyl, any (C₆-C₁₀)aryl and any (4-10)-membered heterocyclyl of the foregoing R^{9a} groups are optionally substituted with 1 to 3 substituents independently selected from the group consisting of halo, cyano, nitro, —CF₃, —CFH₂, —CF₂H, trifluoromethoxy, azido, —OR^{15a}, —(C═O)—R^{15a}, —(C═O)—O—R^{15a}, —O—(C═O)—R^{15a}, —NR^{15a}—(C═O)—R^{16a}, —(C═O)—NR^{15a}R^{16a}, —NR^{15a}R^{16a}, —NR^{15a}—O—R^{16a}, (C₁-C₆)alkyl, (C₂-C₆)alkenyl, (C₂-C₆)alkynyl, —(CR^{17a}R^{18a})ᵤ(C₆-C₁₀)aryl, and —(CR^{17a}R^{18a})ᵤ(4-10)-membered heterocyclyl;
each R^{10a}, R^{11a}, R^{12a}, R^{13a}, R^{14a}, R^{15a}, R^{16a}, R^{17a}, and R^{18a} group is independently selected from the group consisting of H, (C₁-C₆)alkyl, —(CR^{19a}R^{20a})ₚ(C₆-C₁₀)aryl, and —(CR^{19a}R^{20a})ₚ(4-10)-membered heterocyclyl;
any 1 or 2 carbon atoms of the (4-10)-membered heterocyclyl of said each R^{10a}, R^{11a}, R^{12a}, R^{13a}, R^{14a}, R^{15a}, R^{16a}, R^{17a}, and R^{18a} group is optionally substituted with an oxo (═O);
any carbon atom of any (C₁-C₆)alkyl, any (C₆-C₁₀)aryl and any (4-10)-membered heterocyclyl of the foregoing R^{10a}, R^{11a}, R^{12a}, R^{13a}, R^{14a}, R^{15a}, R^{16a}, R^{17a}, and R^{18a} groups are optionally substituted with 1 to 3 substituents independently selected from the group consisting of halo, cyano, nitro, —NR^{21a}R^{22a}, —CF₃, —CHF₂, —CH₂F, trifluoromethoxy, (C₁-C₆)alkyl, (C₂-C₆)alkenyl, (C₂-C₆)alkynyl, hydroxy, and (C₁-C₆)alkoxy;
each R^{19a}, R^{20a}, R^{21a}, and R^{22a} group is independently selected from H and (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O, or S atom optionally bears on said group a substituent independently selected from the group consisting of hydroxy, halo, (C₁-C₆)alkyl, (C₁-C₆)alkoxy, amino, —NH(C₁-C₆)(alkyl) or —N((C₁-C₆)(alkyl))₂;
or a pharmaceutically acceptable salt or solvate thereof.

In an embodiment, the invention relates to a compound according to formula (II), wherein W is

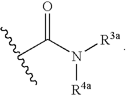

In another embodiment, the invention relates to a compound according to formula (II), wherein W is

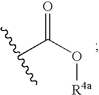

In yet another embodiment, the invention relates to a compound according to formula (II), wherein W is a 5-membered heterocyclyl.

In yet another embodiment, the invention relates to a compound according to formula (II), wherein said 5-membered heterocyclyl is selected from the group consisting of oxazolyl, thiazolyl, pyrazolyl, triazolyl, and oxadiazolyl.

In another embodiment, the invention relates to a compound according to formula (II), wherein b is 2.

In another embodiment, the invention relates to a compound according to formula (II), wherein T is a 6-membered heterocyclyl containing at least one nitrogen atom.

In another embodiment, the invention relates to a compound according to formula (II), wherein said 6-membered heterocyclyl is selected from the group consisting of

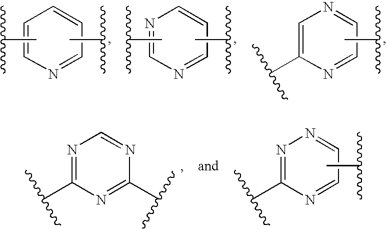

In yet another embodiment, the invention relates to a compound according to formula (II), wherein T^{aa} is

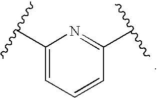

In yet another embodiment, the invention relates to a compound according to formula (II), wherein each R^{1a} is phenyl or napthyl substituted by 1 to 5 R^{9a} groups; wherein: each R^{9a} is independently selected from the group consisting of halo, cyano, —CF₃, hydroxy, (C₁-C₆)alkoxy, (C₁-C₆)alkyl, (C₂-C₆)alkenyl, —(C═O)—R^{10a}, —(C═O)—O—R^{10a}, —O—(C═O)—R^{11a}, —O—(C═O)—R^{11a}, —NR^{11a}—(C═O)—R^{12a}, —(C═O)—NR^{11a}R^{12a}, —NR^{11a}R^{12a}, and —NR^{11a}OR¹².

In an embodiment, the invention relates to a compound according to formula (II), wherein R^{3a} and R^{4a} are each independently selected from H and (C₁-C₆)alkyl;

wherein:

said (C₁-C₆) alkyl is optionally substituted by (C₂-C₆) alkenyl or —(CR^{7a}R^{8a})ₜ(C₃-C₁₀)cycloalkyl.

In another embodiment, the invention relates to a compound according to formula (II), wherein R^{3a} and R^{4a} are taken together with the nitrogen to which they are attached to form a (4-10)-membered heterocyclyl.

In yet another embodiment, the invention relates to a compound according to formula (II), wherein said (4-10)-membered heterocyclyl is selected from the group consisting of:

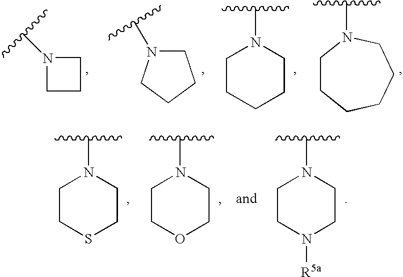

In another embodiment, the invention relates to a compound according to formula (II), wherein R^{4a} is (C₁-C₆)alkyl.

In an embodiment, the invention relates to a compound according to formula (II), wherein n is 0 and at least one of R^{5a} and R^{6a} is H.

In another embodiment, the invention relates to a compound selected from the group consisting of: 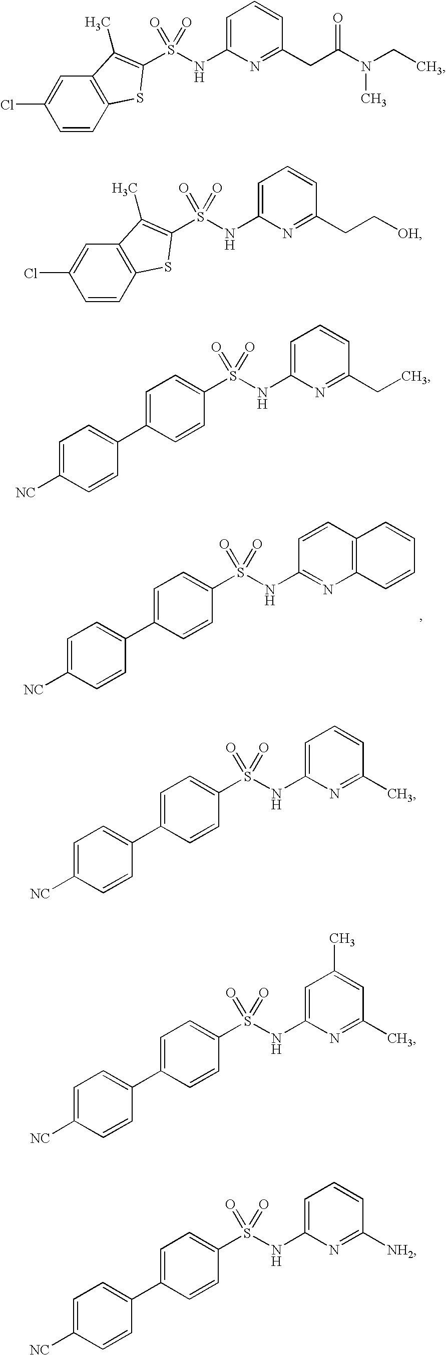 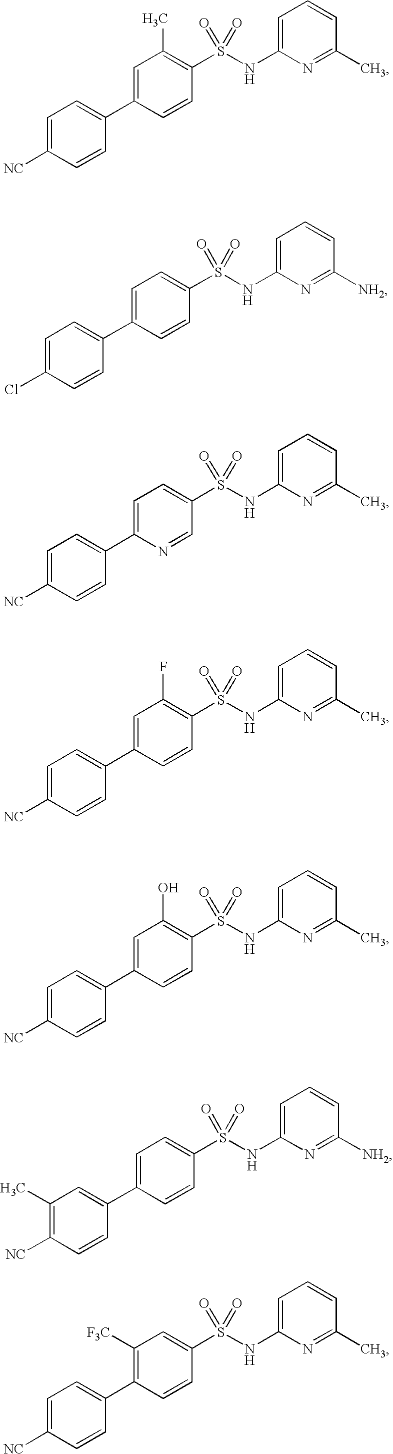 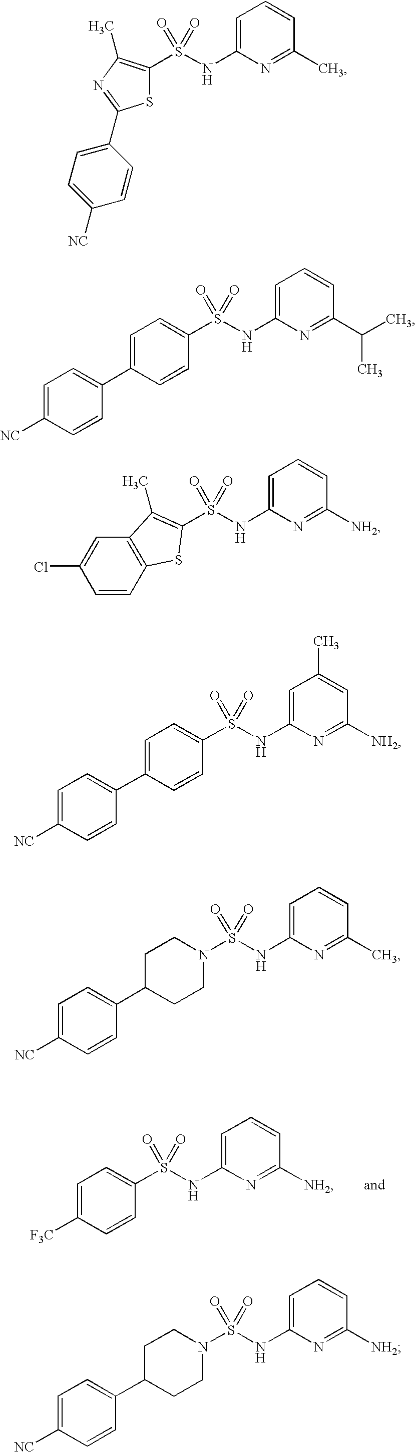
or a pharmaceutically acceptable salt or solvate thereof.

An embodiment of the invention is a compound of formula 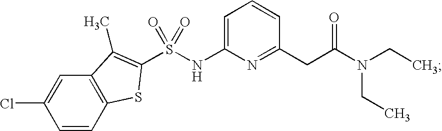
or a pharmaceutically acceptable salt thereof.

In another embodiment of the invention is a compound of formula 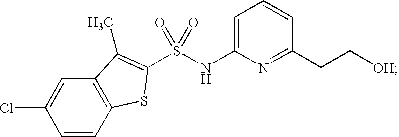
or a pharmaceutically acceptable salt thereof.

In another embodiment of the invention including is a compound of formula 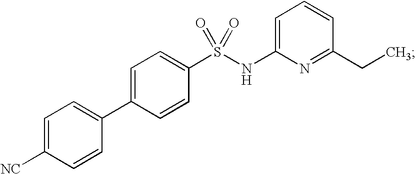
or a pharmaceutically acceptable salt thereof.

In yet another embodiment of the invention is a compound of formula 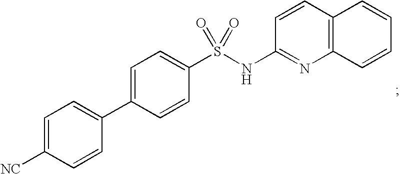
or a pharmaceutically acceptable salt thereof.

In yet another embodiment of the invention is a compound of formula 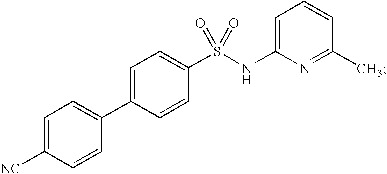
or a pharmaceutical acceptable salt thereof.

An embodiment of the invention is a compound of formula 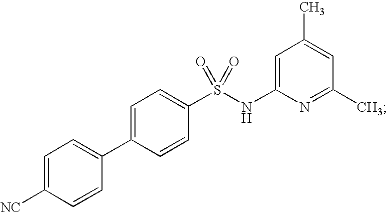
or a pharmaceutically acceptable salt thereof.

In another embodiment of the invention is a compound of formula 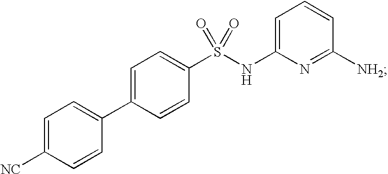
or a pharmaceutical acceptable salt thereof.

In yet another embodiment of the invention is a compound of formula 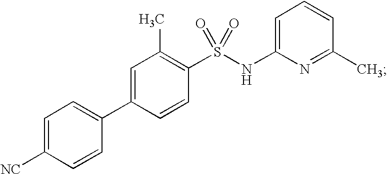
or a pharmaceutically acceptable salt thereof.

In yet another embodiment of the invention is a compound of formula 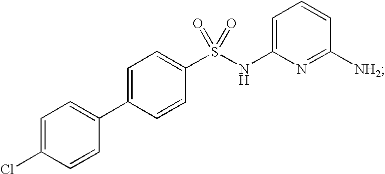
or a pharmaceutically acceptable salt thereof.

In yet another embodiment of the invention is a compound of formula 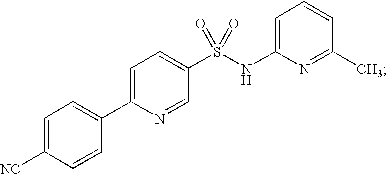
or a pharmaceutically acceptable salt thereof.

An embodiment of the invention is a compound of formula 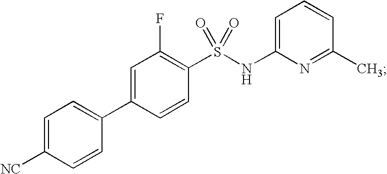
or a pharmaceutical acceptable salt thereof.

In another embodiment of the invention is a compound of formula 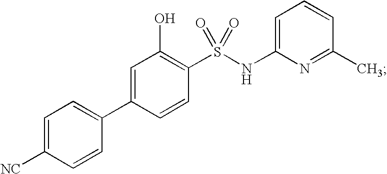
or a pharmaceutically acceptable salt thereof.

In yet another embodiment of the invention is a compound of formula 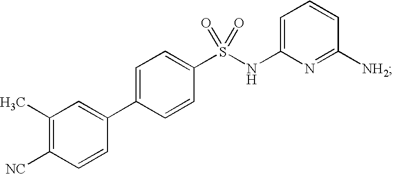
or a pharmaceutically acceptable salt thereof.

In yet another embodiment of the invention is a compound of formula 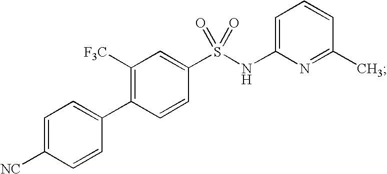
or a pharmaceutically acceptable salt thereof.

In yet another embodiment of the invention is a compound of formula 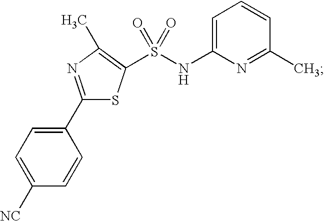
or a pharmaceutically acceptable salt thereof.

An embodiment of the invention is a compound of formula 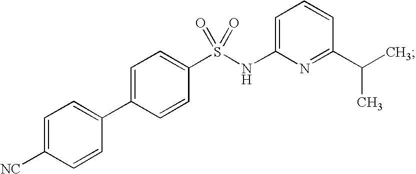
or a pharmaceutically acceptable salt thereof.

In another embodiment of the invention is a compound of formula 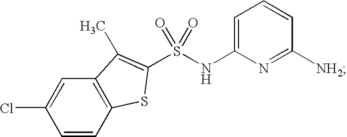
or a pharmaceutically acceptable salt thereof.

In yet another embodiment of the invention is a compound of formula 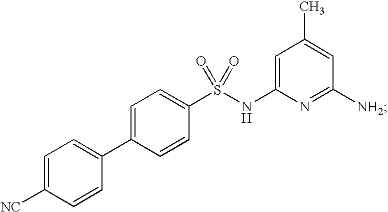
or a pharmaceutically acceptable salt thereof.

In yet another embodiment of the invention is a compound of formula 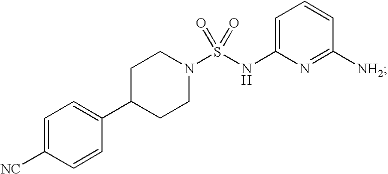
or a pharmaceutically acceptable salt thereof.

In yet another embodiment of the invention is a compound of formula 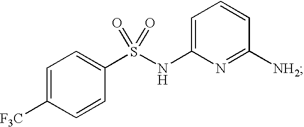
or a pharmaceutically acceptable salt thereof.

In another embodiment of the invention is a compound of formula 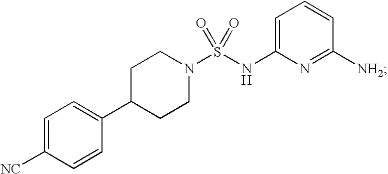
or a pharmaceutically acceptable salt thereof.

An embodiment of the invention relates to a compound of formula (III): 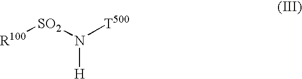
wherein:
R¹⁰⁰ is selected from the group consisting of benzothiophenyl, phenyl, pyridinyl, piperidinyl, and thiazolyl;
the carbon atoms of R¹⁰⁰ may be optionally substituted by 1 to 3 R³⁰⁰ groups;
R³⁰⁰ is selected from the group consisting of hydroxy, (C₁-C₃)alkyl, phenyl, halo, and —CF₃;
the phenyl of the foregoing R³⁰⁰ group may be optionally substituted with 1 to 3 substituents independently selected from the group consisting of halo, cyano, and (C₁-C₃)alkyl;
T⁵⁰⁰ is selected from pyridinyl or quinolinyl;
the carbon atoms of T⁵⁰⁰ may be optionally substituted by 1 to 3 R⁴⁰⁰ groups;
R⁴⁰⁰ is selected from the group consisting of CH₂CH₂—OH, —NH₂, (C₁-C₃)alkyl, and —(C₁-C₃)alkyl-(C═O)—N((C₁-C₃)alkyl)₂;
or a pharmaceutically acceptable salt thereof.

Another embodiment of the invention relates to a compound according to formula (III), wherein R⁴⁰⁰ is —NH_{2.}

In yet another embodiment of the invention, a compound according formula (III), wherein R⁴⁰⁰ is —CH_{3.}

An embodiment of the invention relates to a pharmaceutical composition comprising an effective amount of a compound according formula (I) or formula (II), or a pharmaceutically acceptable salt or solvate thereof, and a pharmaceutically acceptable carrier.

In yet another embodiment, the invention relates to a method of treating a condition that is mediated by the modulation of 11-β-hsd-1, the method comprising administering to a mammal an effective amount of a compound according formula (I), formula (II), or formula (III) or a pharmaceutically acceptable salt or solvate thereof.

In yet another embodiment, the invention relates to a method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which the liver is a target organ, the method comprising administering to a mammal an effective amount of a compound according to formula (I), formula (II), or formula (III) or a pharmaceutically acceptable salt or solvate thereof.

### DEFINITIONS

As used herein, the terms “comprising” and “including” are used in their open, non-limiting sense.

The term “alkyl”, as used herein, unless otherwise indicated, includes saturated monovalent hydrocarbon radicals having straight or branched moieties.

The term “alkenyl”, as used herein, unless otherwise indicated, includes alkyl moieties having at least one carbon-carbon double bond wherein alkyl is as defined above and including E and Z isomers of said alkenyl moiety.

The term “alkynyl”, as used herein, unless otherwise indicated, includes alkyl moieties having at least one carbon-carbon triple bond wherein alkyl is as defined above.

The term “alkoxy”, as used herein, unless otherwise indicated, includes O-alkyl groups wherein alkyl is as defined above.

The term “amino”, as used herein, is intended to include the —NH₂ radical, and any substitutions of the N atom.

The terms “halogen” and “halo,” as used herein represent chlorine, fluorine, bromine or iodine.

The term “trifluoromethyl,” as used herein, is meant to represent a —CF₃ group.

The term “trifluoromethoxy,” as used herein, is meant to represent a —OCF₃ group.

The term “cyano,” as used herein, is meant to represent a —CN group.

The term, “OMs” as used herein, is intended to mean, unless otherwise indicated methanesulfonate.

The term “Me” as used herein, unless otherwise indicated, is intended to mean means methyl.

The term “MeOH” as used herein, unless otherwise indicated, is intended to mean means methanol.

The term “Et” as used herein, unless otherwise indicated, is intended to mean means ethyl.

The term “Et₂O” as used herein, unless otherwise indicated, is intended to mean means diethylether.

The term “EtOH” as used herein, unless otherwise indicated, is intended to mean means ethanol.

The term “Et₃N” as used herein, unless otherwise indicated, is intended to mean means triethylamine.

The term “EtOAc” as used herein, unless otherwise indicated, is ethyl acetate.

The term “AlMe₂Cl” as used herein, unless otherwise indicated, is intended to mean dimethyl aluminum chloride.

The term “Ph” as used herein, unless otherwise indicated, is intended to mean phenyl.

The term “Ac” as used herein, unless otherwise indicated, is intended to mean means acetyl.

The term “TFA” as used herein, unless otherwise indicated, is intended to mean trifluoroacetic acid.

The term “TEA”, as used herein, unless otherwise indicated, is intended to mean triethanolamine.

The term “HATU”, as used herein, unless otherwise indicated, is intended to mean N,N,N′,N′-tetramethyluronium hexafluorophosphate.

The term “THF”, as used herein, unless otherwise indicated, is intended to mean tetrahydrofuran.

The term “TIOH”, as used herein, unless otherwise indicated, is intended to mean thallium(I) hydroxide.

The term “TIOEt”, as used herein, unless otherwise indicated, is intended to mean thallium(I) ethoxide.

The term “PCy₃” as used herein, is intended to mean tricyclohexylphosphine.

The term “Pd₂(dba)₃”, as used herein, unless otherwise indicated, is intended to mean tris(dibenzylideneacetone)dipalladium(0).

The term “Pd(OAc)₂”, as used herein, unless otherwise indicated, is intended to mean palladium(II) acetate.

The term “Pd(PPh₃)₂Cl₂”, as used herein, unless otherwise indicated, is intended to mean dichlorobis(triphenylphosphine)palladium(II).

The term “Pd(PPh₃)₄”, as used herein, unless otherwise indicated, is intended to mean tetrakis(triphenylphophine)palladium(0).

The term “Pd(dpp)Cl₂” as used herein, is intended to mean (1,1′-bis(diphenylphosphino)ferrocene)dichloropalladium(II), complex with dichloromethane (1:1).

The term “G6P”, as used herein, unless otherwise indicated, is intended to mean glucose-6-phosphate.

The term “NIDDM, as used herein, unless otherwise indicated, is intended to mean non insulin dependent diabetes mellitus.

The term “NAHMDS”, as used herein unless otherwise indicated, is intended to mean sodium bis(trimethylsilyl)amide.

The term “NADPH”, as used herein, unless otherwise indicated, is intended to mean nicotinamide adenine dinucleotide phosphate, reduced form.

The term “CDCl₃ or CHLORFORM-D” as used herein, is intended to mean deuterochloroform.

The term “CD₃OD” as used herein, is intended to mean deuteromethanol.

The term “CD₃CN” as used herein, is intended to mean deuteroacetonitrile.

The term “DEAD” as used herein, is intended to mean diethyl azodicarboxylate.

The term “TsCH₂NC” as used herein, is intended to mean tosylmethyl isocyanide.

The term “CISO₃H” as used herein, is intended to mean chlorosulfonic acid.

The term “DMSO-d₆ or DMSO-D₆” as used herein, is intended to mean deuterodimethyl sulfoxide.

The term “DME” as used herein, is intended to mean 1,2-dimethoxyethane.

The term “DMF” as used herein, is intended to mean N,N-dimethylformamide.

The term “DMSO”, as used herein, is intended to mean, unless otherwise indicated dimethylsulfoxide.

The term “DI”, as used herein, is intended to mean deionized.

The term “KOAc” as used herein, is intended to mean potassium acetate.

The term “neat” as used herein, is meant to represent an absence of solvent.

The term “mmol” as used herein, is intended to mean millimole.

The term “equiv” as used herein, is intended to mean equivalent.

The term “mL” as used herein, is intended to mean milliliter.

The term “U” as used herein, is intended to mean units.

The term “mm” as used herein, is intended to mean millimeter.

The term “g” as used herein, is intended to mean gram.

The term “kg” as used herein, is intended to mean kilogram.

The term “h” as used herein, is intended to mean hour.

The term “min” as used herein, is intended to mean minute.

The term “μL” as used herein, is intended to mean microliter.

The term “μM” as used herein, is intended to mean micromolar.

The term “μm” as used herein, is intended to mean micrometer.

The term “M” as used herein, is intended to mean molar.

The term “N” as used herein, is intended to mean normal.

The term “nm” as used herein, is intended to mean nanometer.

The term “nM” as used herein, is intended to mean nanoMolar.

The term “amu” as used herein, is intended to mean atomic mass unit.

The term “° C.” as used herein, is intended to mean Celsius.

The term “m/z, as used herein, is intended to mean, unless otherwise indicated, mass/charge ratio.

The term “wt/wt” as used herein, is intended to mean weight/weight.

The term “v/v” as used herein, is intended to mean volume/volume.

The term “mL/min” as used herein, is intended to mean milliliter/minute.

The term “UV” as used herein, is intended to mean ultraviolet.

The term “APCI-MS” as used herein, is intended to mean atmospheric pressure chemical ionization mass spectroscopy.

The term “HPLC” as used herein, is intended to mean high performance liquid chromatograph.

The term “LC” as used herein, is intended to mean liquid chromatograph.

The term “LCMS” as used herein, is intended to mean liquid chromatography mass spectroscopy.

The term “SFC” as used herein, is intended to mean supercritical fluid chromatography.

The term “sat” as used herein, is intended to mean saturated.

The term “aq” as used herein, is intended to mean aqueous.

The term “ELSD” as used herein, is intended to mean evaporative light scattering detection.

The term “MS” as used herein, is intended to mean mass spectroscopy.

The term “HRMS (ESI)” as used herein, is intended to mean high resolution mass spectrometry (electrospray ionization).

The term “Anal.” as used herein, is intended to mean analytical.

The term “Calcd”, as used herein, is intended to mean calculated.

The term “NT”, as used herein, unless otherwise indicated, is intended to mean not tested.

The term “NA”, as used herein, unless otherwise indicated, is intended to mean not tested.

The term “RT”, as used herein, unless otherwise indicated, is intended to mean room temperature.

The term “Mth.”, as used herein, unless otherwise indicated, is intended to mean Method.

The term “Celite®”, as used herein, unless otherwise indicated, is intended to mean a white solid diatomite filter agent commercially available from World Minerals located in Los Angeles, Calif. USA.

The term “Eg.”, as used herein, unless otherwise indicated, is intended to mean example.

Terms such as —(CR³R⁴)ₜ or —(CR¹⁰R¹¹)ᵥ, for example, are used, R³, R⁴, R¹⁰ and R¹¹ may vary with each iteration of t or v above 1. For instance, where t or v is 2 the terms —(CR³R⁴)ᵥ or —(CR¹⁰OR¹¹)ₜ may equal —CH₂CH₂—, or —CH(CH₃)C(CH₂CH₃)(CH₂CH₂CH₃)—, or any number of similar moieties falling within the scope of the definitions of R³, R⁴, R¹⁰ and R¹¹.

The term “Kⱼ”, as used herein, is intended to mean values of enzyme inhibition constant.

The term “Kᵢ” app, as used herein, is intended to mean Kᵢ apparent.

The term “IC₅₀”, as used herein, is intended to mean concentrations required for at least 50% enzyme inhibition.

The term “substituted,” means that the specified group or moiety bears one or more substituents. The term “unsubstituted,” means that the specified group bears no substituents.

The term “optionally substituted” means that the specified group is unsubstituted or substituted by one or more substituents.

In accordance with convention, in some structural formula herein, the carbon atoms and their bound hydrogen atoms are not explicitly depicted e.g., 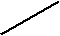
represents a methyl group, 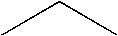
represents an ethyl group, 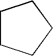
represents a cyclopentyl group, etc.

The term “cycloalkyl”, as used herein, unless otherwise indicated, refers to a non-aromatic, saturated or partially saturated, monocyclic or fused, spiro or unfused bicyclic or tricyclic hydrocarbon referred to herein containing a total of from 3 to 10 carbon atoms, suitably 5-8 ring carbon atoms. Exemplary cycloalkyls include rings having from 3-10 carbon atoms, such as cyclopropyl, cyclobutyl, cyclopentyl, cyclohexyl, cycloheptyl, and adamantyl. Illustrative examples of cycloalkyl are derived from, but not limited to, the following:

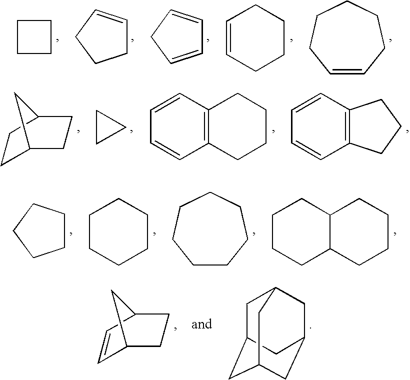

The term “aryl”, as used herein, unless otherwise indicated, includes an organic radical derived from an aromatic hydrocarbon by removal of one hydrogen, such as phenyl or naphthyl.

The term “(3-7)-membered heterocyclyl”, “(6-10)-membered heterocyclyl”, or “(4-10)-member heterocyclyl”, as used herein, unless otherwise indicated, includes aromatic and non-aromatic heterocyclic groups containing one to four heteroatoms each selected from O, S and N, wherein each heterocyclic group has from 3-7, 6-10, or 4-10 atoms, respectively, in its ring system, and with the proviso that the ring of said group does not contain two adjacent O or S atoms. Non-aromatic heterocyclic groups include groups having only 3 atoms in their ring system, but aromatic heterocyclic groups must have at least 5 atoms in their ring system. The heterocyclic groups include benzo-fused ring systems. An example of a 3 membered heterocyclic group is aziridine, an example of a 4 membered heterocyclic group is azetidinyl (derived from azetidine). An example of a 5 membered heterocyclic group is thiazolyl, an example of a 7 membered ring is azepinyl, and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following:

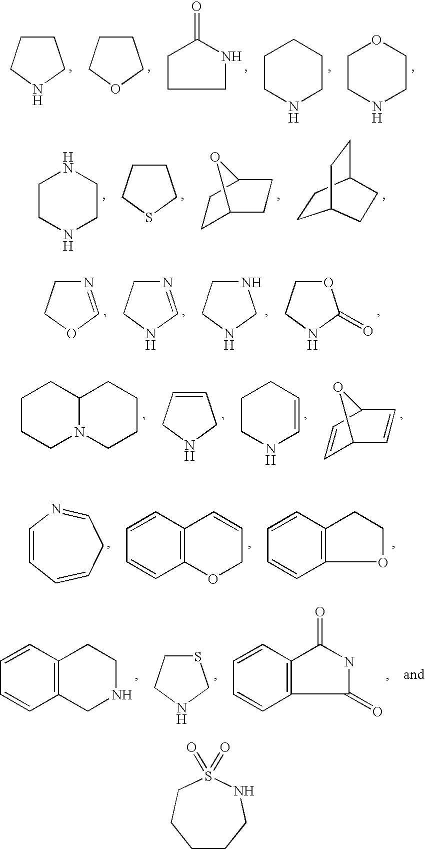

Unless otherwise indicated, the term “oxo” refers to ═O.

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO (dimethylsulfoxide), ethyl acetate, acetic acid, or ethanolamine.

The phrase “pharmaceutically acceptable salt(s)”, as used herein, unless otherwise indicated, includes salts of acidic or basic groups which may be present in the compounds of formula (I) or formula (II). The compounds of formula (I) or formula (II )that are basic in nature are capable of forming a wide variety of salts with various inorganic and organic acids. The acids that may be used to prepare pharmaceutically acceptable acid addition salts of such basic compounds of formula (I) or formula (II) are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

The term “diseases in which the liver is a target organ”, as used herein, unless otherwise indicated means diabetes, hepatitis, liver cancer, liver fibrosis, and malaria.

The term “Metabolic syndrome”, as used herein, unless otherwise indicated means psoriasis, diabetes mellitus, wound healing, inflammation, neurodegenerative diseases, galactosemia, maple syrup urine disease, phenylketonuria, hypersarcosinemia, thymine uraciluria, sulfinuria, isovaleric acidemia, saccharopinuria, 4-hydroxybutyric aciduria, glucose-6-phosphate dehydrogenase deficiency, and pyruvate dehydrogenase deficiency.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The term “modulate” or “modulating”, as used herein, refers to the ability of a modulator for a member of the steroid/thyroid superfamily to either directly (by binding to the receptor as a ligand) or indirectly (as a precursor for a ligand or an inducer which promotes production of ligand from a precursor) induce expression of gene(s) maintained under hormone expression control, or to repress expression of gene(s) maintained under such control.

The term “obesity” or “obese”, as used herein, refers generally to individuals who are at least about 20-30% over the average weight for his/her age, sex and height. Technically, “obese” is defined, for males, as individuals whose body mass index is greater than 27.8 kg/m², and for females, as individuals whose body mass index is greater than 27.3 kg/m². Those of skill in the art readily recognize that the invention method is not limited to those who fall within the above criteria. Indeed, the method of the invention can also be advantageously practiced by individuals who fall outside of these traditional criteria, for example, by those who may be prone to obesity.

The term “inflammatory disorders”, as used herein, refers to disorders such as rheumatoid arthritis, ankylosing spondylitis, psoriatic arthritis, psoriasis, chondrocalcinosis, gout, inflammatory bowel disease, ulcerative colitis, Crohn's disease, fibromyalgia, and cachexia.

The phrase “therapeutically effective amount”, as used herein, refers to that amount of drug or pharmaceutical agent that will elicit the biological or medical response of a tissue, system, animal, or human that is being sought by a researcher, veterinarian, medical doctor or other.

The phrase “amount . . . effective to lower blood glucose levels”, as used herein, refers to levels of compound sufficient to provide circulating concentrations high enough to accomplish the desired effect. Such a concentration typically falls in the range of about 10 nM up to 2 μM; with concentrations in the range of about 100 nM up to 500 nM being preferred. As noted previously, since the activity of different compounds which fall within the definition of formula (I) or formula (II) as set forth above may vary considerably, and since individual subjects may present a wide variation in severity of symptoms, it is up to the practitioner to determine a subject's response to treatment and vary the dosages accordingly.

The phrase “insulin resistance”, as used herein, refers to the reduced sensitivity to the actions of insulin in the whole body or individual tissues, such as skeletal muscle tissue, myocardial tissue, fat tissue or liver tissue. Insulin resistance occurs in many individuals with or without diabetes mellitus.

The phrase “insulin resistance syndrome”, as used herein, refers to the cluster of manifestations that include insulin resistance, hyperinsulinemia, NIDDM, arterial hypertension, central (visceral) obesity, and dyslipidemia.

Certain compounds of formula (I) or formula (II) may have asymmetric centers and therefore exist in different enantiomeric forms. All optical isomers and stereoisomers of the compounds of formula (I) or formula (II), and mixtures thereof, are considered to be within the scope of the invention. With respect to the compounds of formula (I) or formula (II), the invention includes the use of a racemate, one or more enantiomeric forms, one or more diastereomeric forms, or mixtures thereof. The compounds of formula (I) or formula (II) may also exist as tautomers. This invention relates to the use of all such tautomers and mixtures thereof.

Certain functional groups contained within the compounds of the present invention can be substituted for bioisosteric groups, that is, groups which have similar spatial or electronic requirements to the parent group, but exhibit differing or improved physicochemical or other properties. Suitable examples are well known to those of skill in the art, and include, but are not limited to moieties described in Patini et al., Chem. Rev, 1996, 96, 3147-3176 and references cited therein.

The subject invention also includes isotopically-labelled compounds, which are identical to those recited in formula (I) or formula (II), but for the fact that one or more atoms are replaced by an atom having an atomic mass or mass number different from the atomic mass or mass number, usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention and pharmaceutically acceptable salts or solvates of said compounds which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula (I) or formula (II) of this invention thereof can generally be prepared by carrying out the procedures disclosed in the Schemes and/or in the Examples below, by substituting a readily available isotopically labeled reagent for a non-isotopically labeled reagent.

Other aspects, advantages, and features of the invention will become apparent from the detailed description below.

### DETAILED DESCRIPTION AND EMBODIMENTS OF THE INVENTION

The following reaction Schemes illustrate the preparation of the compounds of the present invention. Unless otherwise indicated, R¹—R²¹; R^{1a}—R^{22a}, T, T^{aa}, and W in the reaction schemes and the discussion that follows are as defined above.

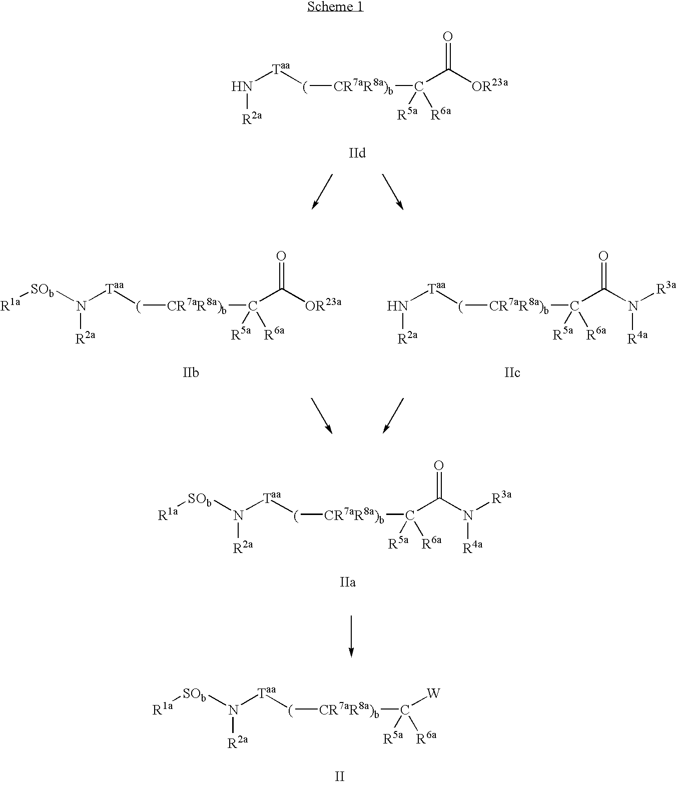

Referring to Scheme 1 above, the compound of formula IIa may be prepared by reacting a compound of formula IIb, wherein the group CO₂R^{23a} (wherein R^{23a} is selected from H and (C₁-C₆)alkyl) is an ester group such as methyl ester (CO₂—CH₃) or ethyl ester (CO₂—CH₂CH₃), with aluminum amides (Me₂Al—NR^{3a}R^{4a}) or (MeAl(Cl)—NR^{3a}R^{4a}) in a suitable solvent (e.g. dichloromethane or toluene) advantageously, from room temperature to the boiling point of the solvent, typically from about 20 degrees Celsius to about 100 degrees Celsius. The compound of formula IIa may also be prepared by reacting a compound of formula IIb, wherein the group CO₂R^{23a} is a carboxylic acid (CO₂H) with an amine of formula HNR^{3a}R^{4a} using standard amide coupling chemistry. Compounds of formula IIb may be prepared by reacting a compound of formula IId, wherein the group CO₂R^{23a} is an ester group such as methyl ester (CO₂—CH₃) or ethyl ester (CO₂—CH₂CH₃), with a R^{1a}-sulfonyl halide or R^{1a}-sulfinyl halide. Alternatively, the compound of formula IIa may be prepared by reacting a compound of formula IIc with a R^{1a}-sulfonyl halide or R^{1a}-sulfinyl halide. Compounds of formula IIc may be prepared by reacting a compound of formula IId, wherein the group CO₂R^{23a} is an ester group such as methyl ester (CO₂—CH₃) or ethyl ester (CO₂—CH₂CH₃), with aluminum amides (Me₂Al—NR^{3a}R^{4a}) or (MeAl(Cl)—NR^{3a}R^{4a}) in a suitable solvent (e.g. dichloromethane or toluene) at a temperature from room temperature to the boiling point of the solvent, typically from about 20 degrees Celsius to about 100 degrees Celsius. The compound of formula II may be obtained by cyclodehydration of suitable amide IIa.

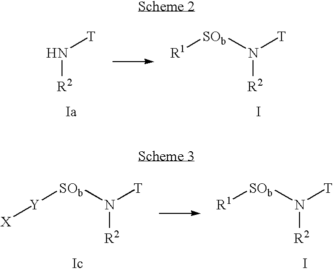

Referring to Scheme 2 above, the compound of formula I may be prepared by reacting la with an R¹-sulfonyl halide, R¹-sulfinyl halide, or R¹-sulfinate in the presence of a base such as an amine. Suitable bases include pyridine, triethylamine, and diisopropylethylamine. Suitable solvents include pyridine, dichloromethane, or THF. The aforementioned reaction can be conducted at room temperature or heated for an appropriate time period, such as 2 to 16 hours, depending on the solvent system used. After the reaction is substantially completed, the base may be removed in vacuo and the resulting residue may be purified using conventional purification techniques.

Referring to Scheme 3, an alternative method of synthesis is shown for compounds where R¹ is a non-fused ring system of more than one ring of either an aryl or heterocyclyl. The compound of formula I, may be prepared by a palladium-catalyzed coupling reaction of Ic where X is a halo or trifluoromethylsulfonyl with a reagent Y—N where Y is aryl or heterocyclyl, N is boronic acid, boronate ester, stannane, or zincate. Suitable palladium sources for this reaction include Pd(PPh₃)₄, Pd₂(dba)₃, Pd(PPh₃)₂Cl₂ or Pd(OAc)₂. Ligands such as diphenylphosphinoethane, diphenylphosphinoferrocene, or triphenylphosphine may also be added. Suitable solvents for the palladium-catalyzed coupling reaction include dimethylformamide, tetrahydrofuran, or toluene. The aforementioned reaction can be conducted at a temperature of about 50° C. to about 150° C. with or without microwave heating for a time period of about 15 min to about 16 hours. For couplings using boronic acids, base additives such as Na₂CO₃, Cs₂CO₃, TIOH, TIOEt may be added.

Any of the above compounds of formula IIa, IIb, IIc, IId, II, I, Ia and Ic can be converted into another analogous compound by standard chemical manipulations. All starting materials, regents, and solvents are commercially available and are known to those of skill in the art unless otherwise stated. These chemical manipulations are known to those skilled in the art and include (a) removal of a protecting group by methods outlined in T. W. Greene and P. G. M. Wuts, “Protective Groups in Organic Synthesis”, Second Edition, John Wiley and Sons, New York, 1991; (b) displacement of a leaving group (halide, risylate, tosylate, etc) with a primary or secondary amine, thiol or alcohol to form a secondary or tertiary amine, thioether or ether, respectively; (c) treatment of primary and secondary amines with an isocyanate, acid chloride (or other activated carboxylic acid derivative), alkyl/aryl chloroformate or sulfonyl chloride to provide the corresponding urea, amide, carbamate or sulfonamide; (d) reductive amination of a primary or secondary amine using an aldehyde.

The compounds of the present invention may have asymmetric carbon atoms. Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

The compounds of formula (I) or formula (II) that are basic in nature are capable of forming a wide variety of different salts with various inorganic and organic acids. Although such salts must be pharmaceutically acceptable for administration to animals, it is often desirable in practice to initially isolate the compound of formula (I) or formula (II) from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula (I) or formula (II) that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula (I) or formula (II). Such non-toxic base salts include those derived from such pharmacologically acceptable cations as sodium, potassium, calcium, and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The compounds of the present invention may be modulators of 11-β-hsd-1. The compounds of the present invention may modulate processes mediated by 11-β-hsd-1, which refer to biological, physiological, endocrinological, and other bodily processes which are mediated by receptor or receptor combinations which are responsive to the 11-β-hsd-1 inhibitors described herein (e.g., diabetes, hyperlipidemia, obesity, impaired glucose tolerance, hypertension, fatty liver, diabetic complications (e.g. retinopathy, nephropathy, neurosis, cataracts and coronary artery diseases and the like), arteriosclerosis, pregnancy diabetes, polycystic ovary syndrome, cardiovascular diseases (e.g. ischemic heart disease and the like), cell injury (e.g.) brain injury induced by strokes and the like) induced by atherosclerosis or ischemic heart disease, gout, inflammatory diseases (e.g. arthrosteitis, pain, pyrexia, rheumatoid arthritis, inflammatory enteritis, acne, sunburn, psoriasis, eczema, allergosis, asthma, GI ulcer, cachexia, autoimmune diseases, pancreatitis and the like), cancer, osteoporosis and cataracts. Modulation of such processes can be accomplished in vitro or in vivo. In vivo modulation can be carried out in a wide range of subjects, such as, for example, humans, rodents, sheep, pigs, cows, and the like.

The compounds, according to the present invention may be used in several indications which involve modulations of 11-β-hsd-1 enzyme. Thus, the compounds according to the present invention may be used against dementia (See WO97/07789), osteoporosis (See Canalis E 1996, “Mechanisms of Glucocorticoid Action in Bone: Implications to Glucocorticoid-Induced Osteoporosis”, Journal of Clinical Endocrinology and Metabolism, 81, 3441-3447) and may also be used disorders in the immune system (see Franchimont, et. al, “Inhibition of Th1 Immune Response by Glucocorticoids: Dexamethasone Selectively Inhibits IL-12-induced Stat 4 Phosphorylation in T Lymphocytes”, The Journal of Immunology 2000, February 15, vol 164 (4), pages 1768-74) and also in the above listed indications.

Inhibition of 11-β-hsd-1 in isolated murine pancreatic β-cells improves the glucose-stimulated insulin secretion (Davani, B., et al. (2000) J. Biol. Chem. Nov. 10, 2000; 275(45): 34841-4). Glucocorticoids were previously known to reduce pancreatic insulin release in vivo (Billaudel, B. and B. C. J. Sutter (1979) Horm. Metab. Res. 11: 555-560). Thus, inhibition of 11-β-hsd-1 is predicted to yield other beneficial effects for diabetes treatment, besides effects on liver and fat.

Recent data suggests that the levels of the glucocorticoid target receptors and the 11-β-hsd-1 enzymes determine the susceptibility to glaucoma (Stokes, J., et al., (2000) Invest. Ophthalmol. 41:1629-1638). Further, inhibition of 11-β-hsd-1 was recently presented as a novel approach to lower the intraocular pressure (Walker E. A., et al, poster P3-698 at the Endocrine society meeting Jun. 12-15, 1999, San Diego). Ingestion of carbenoxolone, a non-specific inhibitor of 11-β-hsd-1, was shown to reduce the intraocular pressure by 20% in normal subjects. In the eye, expression of 11-β-hsd-1 is confined to basal cells of the corneal epithelium and the non-pigmented epithelialium of the cornea (the site of aqueous production), to ciliary muscle and to the sphincter and dilator muscles of the iris. In contrast, the distant isoenzyme 11 beta-hydroxysteroid dehydrogenase type 2 is highly expressed in the non-pigmented ciliary epithelium and corneal endothelium. None of the enzymes is found at the trabecular meshwork, the site of drainage. Thus, 11-β-hsd-1 is suggested to have a role in aqueous production, rather than drainage, but it is presently unknown if this is by interfering with activation of the glucocorticoid or the mineralocorticoid receptor, or both.

Bile acids inhibit 11-β-hydroxysteroid dehydrogenase type 2. This results in a shift in the overall body balance in favor of cortisol over cortisone, as shown by studying the ratio of the urinary metabolites (Quattropani C, Vogt B, Odermatt A, Dick B, Frey B M, Frey F J. 2001. J Clin Invest. Nov; 108(9): 1299-305. “Reduced Activity of 11-beta-hydroxysteroid dehydrogenase in Patients with Cholestasis”). Reducing the activity of 11-β-hsd-1 in the liver by a selective inhibitor is predicted to reverse this imbalance, and acutely counter the symptoms such as hypertension, while awaiting surgical treatment removing the biliary obstruction.

The compounds of the present invention may also be useful in the treatment of other metabolic disorders associated with impaired glucose utilization and insulin resistance include major late-stage complications of NIDDM, such as diabetic angiopathy, atherosclerosis, diabetic nephropathy, diabetic neuropathy, and diabetic ocular complications such as retinopathy, cataract formation and glaucoma, and many other conditions linked to NIDDM, including dyslipidemia glucocorticoid induced insulin resistance, dyslipidemia, polycysitic ovarian syndrome, obesity, hyperglycemia, hyperlipidemia, hypercholesteremia, hypertriglyceridemia, hyperinsulinemia, and hypertension. Brief definitions of these conditions are available in any medical dictionary, for instance, Stedman's Medical Dictionary (10^{th} Ed.).

### Assay

The 11β-hsd-1 assay was performed in a 100 mM Triethanolamine buffer pH 8.0, containing 200 mM NaCl, 0.02% n-dodecyl β-D-maltoside, 5% glycerol, 5 mM β-mercaptoethanol. A typical reaction for the determination of Kᵢₚₚ values was carried at R.T. in a Corning® u-bottom 96-well plate and is described as follows: 11β-hsd-1 enzyme (5 nM, final concentration) was pre-incubated in the presence of the inhibitor and NADPH (500 μM, final concentration) for at least 30 minutes in the assay buffer. When pre-incubation was completed, the reaction was initiated by adding the regenerating system (2 mM Glucose-6-Phosphate, 1 U/mL Glucose-6-Phosphate dehydrogenase, and 6 mM MgCl₂, all the concentration reported are final in the assay buffer), and 3H-cortisone (200 nM, final concentration). After 60 minutes, 60 μL of the assay mixture was transferred to a second 96-well plate and mixed with an equal volume of dimethylsulfoxide to stop the reaction. A 15 μL aliquot from the reaction mixture was loaded into a C-18 column (Polaris C18-A, 50×4.6 mm, 5μ, 180 Angstrom from Varian) connected to an automated High-throughput Liquid Chromatography instrument developed by Cohesive Technologies, commercially available from Franklin, Mass. USA, with a β-RAM model 3 Radio-HPLC detector from IN/US, commercially available from Tampa, Fla. USA. The substrate and product peaks were separated by using an isocratic mixture of 43:57 methanol to water (v/v) at a flow rate of 1.0 mL/min.

The initial reaction velocities were measured by stopping the reaction at 60 min and by measuring the area of product formation in the absence and the presence of various concentrations of inhibitors. The Kᵢₐₚₚ values were determined using the equation for tight-binding inhibitor developed by Morrison, J F. (Morrison J F. Biochim Biophys Acta. 1969; 185: 269-86).

The radiolabeled [1,2-3H]-cortisone is commercially available from American Radiolabeled Chemicals Inc of St. Louis, Mo. USA. NADPH, Glucose-6-Phosphate, and Glucose-6-Phosphate dehydrogenase were purchased from Sigma®.

The Kᵢₐₚₚ values of the compounds of the present invention for the 11-β-hsd-1 enzyme may lie typically between about 10 nM and about 10 μM. The compounds of the present invention that were tested all have Kᵢₐₚₚ's in at least one of the above SPA assays of less than 1 μM, preferably less than 100 nM. Certain preferred groups of compounds possess differential selectivity toward the various 11-β-hsd's. One group of preferred compounds possesses selective activity towards 11-β-hsd-1 over 11β-hsd-2. Another preferred group of compounds possesses selective activity towards 11β hsd-2 over 11-β-hsd-1. (Morrison J F. Biochim Biophys Acta. 1969; 185: 269-86).

Percentage of inhibition was determined in a 100 mM Triethanolamine buffer, pH 8.0, 200 mM NaCl, 0.02% n-dodecyl β-D-maltoside and 5 mM β-ME. A typical reaction was carried on a Corning® u-bottom 96-well plate and is described as follows: 11β-hsd-1 enzyme (5 nM, final concentration) was pre-incubated in the presence of the inhibitor and NADPH (500 μM, final concentration) for at least 30 minutes in the assay buffer. When pre-incubation was completed, the reaction was initiated by adding the regenerating system (2 mM Glucose-6-Phosphate, 1 U/mL Glucose-6-Phosphate dehydrogenase, and 6 mM MgCl₂, all the concentration reported are final in the assay buffer), and 3H-cortisone (200 nM, final concentration). After 60 minutes, 60 μL of the assay mixture was transferred to a second 96-well plate and mixed with an equal volume of dimethylsulfoxide to stop the reaction. A 15 μL aliquot from the reaction mixture was loaded into a C-18 column (Polaris C18-A, 50×4.6 mm, 5μ, 180 Angstrom from Varian) connected to an automated High-throughput Liquid Chromatography instrument developed by Cohesive Technologies commercially available from Franklin, Mass., with a β-RAM model 3 Radio-HPLC detector from IN/US commercially available from Tampa, Fla. The substrate and product peaks were separated by using an isocratic mixture of 43:57 methanol to water (v/v) at a flow rate of 1.0 mL/min.

Percent Inhibition was calculated based on the following equation: (100-(3H-Cortisol peak area with inhibitor/3Hcortisol peak area without inhibitor)×100). Certain groups of compounds possess differential selectivity toward the various 11-β-hsd enzymes. One group of compounds possesses selective activity towards 11-β-hsd-1enzyme over 11β-hsd-2 enzyme. While another group of compounds possesses selective activity towards 11β hsd-2 enzymes over 11-β-hsd-1 enzymes.

[1,2-3H]-cortisone is commercially available from American Radiolabeled Chemicals Inc. of St. Louis, Mo. USA. NADPH while Glucose-6-Phosphate and Glucose-6-Phosphate dehydrogenase was purchased from Sigma®.

### Pharmaceutical Compositions/Formulations, Dosaging and Modes of Administration

Methods of preparing various pharmaceutical compositions with a specific amount of active compound are known, or will be apparent, to those skilled in this art. In addition, those of ordinary skill in the art are familiar with formulation and administration techniques. Such topics would be discussed, e.g. in Goodman and Gilman's The Pharmaceutical Basis of Therapeutics, current edition, Pergamon Press; and Remington's Pharmaceutical Sciences, current edition. Mack Publishing, Co., Easton, Pa. These techniques can be employed in appropriate aspects and embodiments of the methods and compositions described herein. The following examples are provided for illustrative purposes only and are not meant to serve as limitations of the present invention.

The compounds of formula (I) or formula (II) may be provided in suitable topical, oral and parenteral pharmaceutical formulations for use in the treatment of 11-β-hsd-1 mediated diseases. The compounds of the present invention may be administered orally as tablets or capsules, as oily or aqueous suspensions, lozenges, troches, powders, granules, emulsions, syrups or elixirs. The compositions for oral use may include one or more agents for flavoring, sweetening, coloring and preserving in order to produce pharmaceutically elegant and palatable preparations. Tablets may contain pharmaceutically acceptable excipients as an aid in the manufacture of such tablets. As is conventional in the art these tablets may be coated with a pharmaceutically acceptable enteric coating, such as glyceryl monostearate or glyceryl distearate, to delay disintegration and absorption in the gastrointestinal tract to provide a sustained action over a longer period.

Formulations for oral use may be in the form of hard gelatin capsules wherein the active ingredient is mixed with an inert solid diluent, for example, calcium carbonate, calcium phosphate or kaolin. They may also be in the form of soft gelatin capsules wherein the active ingredient is mixed with water or an oil medium, such as peanut oil, liquid paraffin or olive oil.

Aqueous suspensions normally contain active ingredients in admixture with excipients suitable for the manufacture of an aqueous suspension. Such excipients may be a suspending agent, such as sodium carboxymethyl cellulose, methyl cellulose, hydroxypropylmethyl cellulose, sodium alginate, polyvinylpyrrolidone, gum tragacanth and gum acacia; a dispersing or wetting agent that may be a naturally occurring phosphatide such as lecithin, a condensation product of ethylene oxide and a long chain fatty acid, for example polyoxyethylene stearate, a condensation product of ethylene oxide and a long chain aliphatic alcohol such as heptadecaethylenoxycetanol, a condensation product of ethylene oxide and a partial ester derived from a fatty acid and hexitol such as polyoxyethylene sorbitol monooleate or a fatty acid hexitol anhydrides such as polyoxyethylene sorbitan monooleate.

The pharmaceutical compositions may be in the form of a sterile injectable aqueous or oleagenous suspension. This suspension may be formulated according to know methods using those suitable dispersing or wetting agents and suspending agents that have been mentioned above. The sterile injectable preparation may also be formulated as a suspension in a non toxic perenterally-acceptable diluent or solvent, for example as a solution in 1,3-butanediol. Among the acceptable vehicles and solvents that may be employed are water, Ringers solution and isotonic sodium chloride solution. For this purpose any bland fixed oil may be employed including synthetic mono- or diglycerides. In addition fatty acids such as oleic acid find use in the preparation of injectables.

The compounds of formula (I) or formula (II) may also be administered in the form of suppositories for rectal administration of the drug. These compositions can be prepared by mixing the drug with a suitable non-irritating excipient that is solid at about 25 Celsius but liquid at rectal temperature and will therefore melt in the rectum to release the drug. Such materials include cocoa butter and other glycerides.

For topical use preparations, for example, creams, ointments, jellies solutions, or suspensions, containing the compounds of the present invention are employed.

The compounds of formula (I) or formula (II) may also be administered in the form of liposome delivery systems such as small unilamellar vesicles, large unilamellar vesicles and multimellar vesicles. Liposomes can be formed from a variety of phospholipides, such as cholesterol, stearylamine or phosphatidylcholines.

Dosage levels of the compounds of the present invention are of the order of about 0.5 mg/kg body weight to about 100 mg/kg body weight. A preferred dosage rate is between about 30 mg/kg body weight to about 100 mg/kg body weight. It will be understood, however, that the specific dose level for any particular patient will depend upon a number of factors including the activity of the particular compound being administered, the age, body weight, general health, sex, diet, time of administration, route of administration, rate of excretion, drug combination and the severity of the particular disease undergoing therapy. To enhance the therapeutic activity of the present compounds they may be administered concomitantly with other orally active antidiabetic compounds such as the sulfonylureas, for example, tolbutamide and the like.

### Ocular/Aural Administration

For administration to the eye, a compound of the present invention is delivered in a pharmaceutically acceptable ophthalmic vehicle such that the compound is maintained in contact with the ocular surface for a sufficient time period to allow the compound to penetrate the cornea and/or sclera and internal regions of the eye, including, for example, the anterior chamber, posterior chamber, vitreous body, aqueous humor, vitreous humor, cornea, iris/cilary, lens, choroid/retina and sclera. The pharmaceutically acceptable ophthalmic vehicle may be an ointment, vegetable oil, or an encapsulating material. A compound of the invention may also be injected directly into the vitreous humor or aqueous humor.

Further, a compound may be also be administered by well-known, acceptable methods, such as subtebnon and/or subconjunctival injections. The sclera and Tenon's capsule define the exterior surface of the globe of the eye. For treatment of ARMD, CNV, retinopathies, retinitis, uveitis, cystoid macular edema (CME), glaucoma, and other diseases or conditions of the posterior segment of the eye, it is preferable to dispose a depot of a specific quantity of an ophthalmically acceptable pharmaceutically active agent directly on the outer surface of the sclera and below Tenon's capsule. In addition, in cases of ARMD and CME it is most preferable to dispose the depot directly on the outer surface of the sclera, below Tenon's capsule, and generally above the macula.

Alternatively, the active ingredient may be in powder form for constitution with a suitable vehicle, e.g., sterile pyrogen-free water, before use. The compounds may also be formulated in rectal compositions such as suppositories or retention enemas, e.g, containing conventional suppository bases such as cocoa butter or other glycerides.

In addition to the formulations described above, the compounds may also be formulated as a depot preparation. Such long-acting formulations may be administered by implantation (for example, subcutaneously or intramuscularly) intramuscular injection or by the above mentioned subtenon or intravitreal injection.

Within particular embodiments of the invention, the compounds may be prepared for topical administration in saline (combined with any of the preservatives and antimicrobial agents commonly used in ocular preparations), and administered in eyedrop form. The solution or suspension may be prepared in its pure form and administered several times daily. Alternatively, present compositions, prepared as described above, may also be administered directly to the cornea.

Within alternative embodiments, the composition is prepared with a muco-adhesive polymer which binds to cornea. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

Within further embodiments, the present compositions may be utilized as an adjunct to conventional steroid therapy.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5 W) contains VPD diluted 1:1 with a 5% dextrose in water solution. This co-solvent system dissolves hydrophobic compounds well, and itself produces low toxicity upon systemic administration. The proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

### EXAMPLES

The examples and preparations provided below further illustrate and exemplify the compounds of the present invention and methods of preparing such compounds. It is to be understood that the scope of the present invention is not limited in any way by the scope of the following examples and preparations. In the following examples molecules with a single chiral center, unless otherwise noted, exist as a racemic mixture. Those molecules with two or more chiral centers, unless otherwise noted, exist as a racemic mixture of diastereomers. Single enantiomers/diastereomers may be obtained by methods known to those skilled in the art.

The structures of the compounds are confirmed by either elemental analysis or NMR, where peaks assigned to the characteristic protons in the titled compound are presented where appropriate. ¹H NMR shift (δ_{H}) are given in parts per million (pμm) down filed from an internal reference standard.

The invention will now be described in reference to the following EXAMPLES. These EXAMPLES are not to be regarded as limiting the scope of the present invention, but shall only serve in an illustrative manner.

### Method A

### Example 1

### Ethyl [6-(3-Chloro-2-methyl-benzenesulfonylamino)-pyridin-2-yl]-acetate

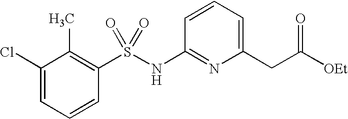

3-Chloro-2-methylbenzenesulfonyl chloride (3.4 g, 15 mmol, 1.5 equiv) was added in one portion to a solution of ethyl (6-amino-pyridin-2-yl)-acetate (Goto, J.; Sakane, K.; Nakai, Y.; Teraji, T.; Kamiya, T J. Antibiot. 1984, 37, 532) (1.8 g, 10 mmol, 1 equiv) in pyridine (75 mL) at 24° C. After 16 hours, the pyridine was removed in vacuo (<1 mm Hg), and the resulting residue was dissolved in ethyl acetate (200 mL). The organic solution was washed sequentially with water (3×100 mL) and saturated aqueous sodium chloride (100 mL). The collected organic was dried over anhydrous sodium sulfate, filtered, and concentrated. Purification by high performance flash chromatography (0→5% methanol in dichloromethane) yielded product (2.76 g, 75%).

### Method B

### Example 8

### 3-Chloro-2-methyl-N-[6-(2-morpholin-4-yl-2-oxo-ethyl)-pyridin-2-yl]-benzenesulfonamide

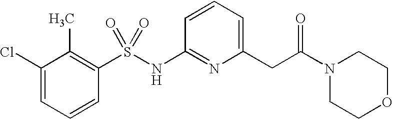

Dimethylaluminum chloride (1.36 mL, 1.36 mmol, 5.0 equiv, 1.0 M in hexanes) was added dropwise to an ice-cooled solution of morpholine (0.119 mL, 1.36 mmol, 5.0 equiv) in dichloromethane (3 mL). The resulting solution was warmed to 24° C. for 1 hour before the addition of a solution of ethyl [6-(3-chloro-2-methyl-benzenesulfonylamino)-pyridin-2-yl]-acetate (0.100 g, 0.271 mmol, 1 equiv) in dichloromethane (2 mL). After 1 hour, 20% sodium potassium tartrate aqueous solution (5 mL) was slowly added to the reaction mixture, and the resulting suspension was stirred vigorously for an additional hour. The resulting mixture was extracted with dichloromethane (2×25 mL). The collected organic was dried over anhydrous sodium sulfate, filtered, and concentrated. Purification by high performance flash chromatography (0→10% methanol in dichloromethane) yielded a light orange solid (0.107 g, 96%).

### Method C

### Example 19

### 2-[6-(5-Chloro-3-methyl-benzo[b]thiophene-2-sulfonylamino)-pyridin-2-yl]-N,N-diethyl-acetamide

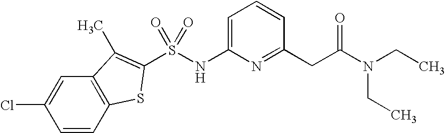

### Preparation of (2-(6-Amino-pyridin-2-yl)-N,N-diethyl-acetamide

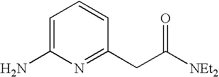

Dimethylaluminum chloride (4.3 mL, 4.3 mmol, 5.0 equiv, 1.0 M solution in hexanes) was added to an ice-cooled solution of diethylamine (445 μL, 4.30 mmol, 5.0 equiv) in dichloromethane (4 mL). After 10 min, the solution was warmed to 24° C. for 1 h. Ethyl (6-amino-pyridin-2-yl)-acetate (Goto, J.; Sakane, K.; Nakai, Y.; Teraji, T.; Kamiya, T. J. Antibiot 1984, 37, 532) (155 mg, 0.860 mmol, 1 equiv) in dichloromethane (4 mL) was added at 24° C. After 21.5 h, potassium sodium tartrate aqueous solution (20% wt/wt, 10 mL) and hexanes (20 mL) were added sequentially, and the resulting mixture was stirred vigorously overnight. Saturated aqueous sodium chloride (30 mL) was added, and the resulting mixture was extracted with ethyl acetate (3×30 mL). The collected organic was,dried over anhydrous sodium sulfate, filtered, and concentrated. Purification by high performance flash chromatography (0→4.5% methanol in dichloromethane+0.1% ammonium hydroxide) provided product (120 mg,67%).¹H NMR (400 MHz, CDCl₃), δ: 7.37 (m, 1 H), 6.66 (d, J=7.6 Hz, 1 H), 6.35 (d, J=8.1 Hz, 1 H), 4.34 (br s, 2 H), 3.69 (s, 2 H), 3.30-3.44 (m, 4 H), 1.06-1.16 (m, 6 H).

### 2-[6-(5-Chloro-3-methyl-benzo[b]thiophene-2-sulfonylamino)-pyridin-2-yl]-N,N-diethyl-acetamide

5-chloro-3-methylbenzo[B]thiophene-2-sulfonyl chloride (163 mg, 0.580 mmol, 1.1 equiv) was added to a solution of 2-(6-amino-pyridin-2-yl)-N,N-diethyl-acetamide (100 mg, 0.483 mmol, 1 equiv) in pyridine (4 mL) at 24° C. After 18 h, the reaction mixture was diluted with ethyl acetate (30 mL). The resulting solution was washed with water (60 mL). The organic phase was dried over anhydrous sodium sulfate, filtered, and concentrated. Purification by high performance flash chromatography (0→5% methanol in dichloromethane) provided the title compound (93 mg, 43%).

### Method D

### Example 26

### [6-(3-Chloro-2-methyl-benzenesulfonylamino)-pyridin-2-yl]-acetic acid

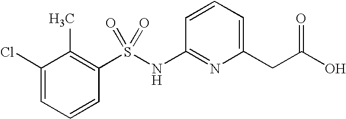

Potassium hydroxide (0.843 g, 15.0 mmol, 6.00 equiv) was added to a solution of [6-(3-chloro-2-methyl-benzenesulfonylamino)-pyridin-2-yl]-acetic acid ethyl ester (0.922 g, 2.50 mmol, 1 equiv) in 20:1 ethanol/water (21 mL) at 24° C. After 1 h, the reaction mixture was concentrated in vacuo (˜25 mm Hg), and the resulting residue was dissolved in water (50 mL). The aqueous solution was acidified by the addition of 10% hydrochloric acid aqueous solution until pH=2. The heterogeneous solution was then filtered, and the solid was rinsed sequentially with water (50 mL) and diethyl ether (2×50 mL). The solid was dried in vacuo (<1 mm Hg, 50° C.) to provide product as a tan solid (0.810 g, 71%).

### Method E

### Example 27

### N-Adamantan-1-yl-2-[6-(3-chloro-2-methyl-benzenesulfonylamino)-pyridin-2-yl]-acetamide

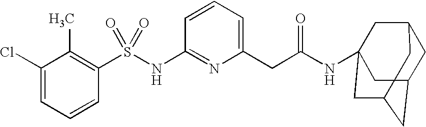

O-(7-Azabenzotriazol-1-yl-N,N,N′,N′-tetramethyluronium hexafluorophosphate (0.11 g, 0.29 mmol, 0.98 equiv) was added in one portion to an ice-cooled solution of [6-(3-chloro-2-methyl-benzenesulfonylamino)-pyridin-2-yl]-acetic acid (0.100 g, 0.293 mmol, 1 equiv), 1-adamantanamine (0.200 g, 1.32 mmol, 4.51 equiv), and N,N-diisopropylethylamine (0.462 mL, 2.65 mmol, 9.04 equiv) in dimethylformamide (5 mL). The solution was warmed to 24° C. and stirred overnight. Dimethylformamide was removed in vacuo (˜1 mm Hg), and the resulting residue was dissolved in dichloromethane (20 mL). The organic was washed sequentially with deionized water (2×20 mL) and saturated aqueous sodium chloride (20 mL). The collected organic was dried over anhydrous sodium sulfate, filtered, and concentrated. Purification of the resulting residue by high performance flash chromatography (0→2% methanol in dichloromethane) yielded desired amide (82 mg, 65%).
Alternative General Method for Amide Coupling

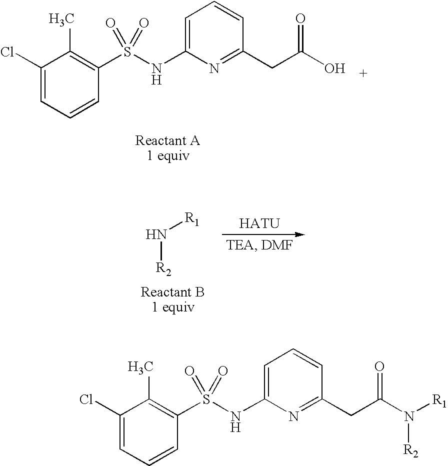

A stir bar, the amine (Reactant B, 400 μL, 80 μmol, 1.00 equiv 0.2 M in anhydrous DMF), [6-(3-chloro-2-methyl-benzenesulfonylamino)-pyridin-2-yl]-acetic acid (Reactant A 200 μL, 80 μmol, 1.00 equiv 0.2 M in anhydrous DMF), TEA (160 μL, 80 μmol, 1.00 equiv 0.5 M in anhydrous DMF), HATU (160 μL, 80 μmol, 1.00 equiv 0.5 M in anhydrous DMF) were placed into a 10×75 mm test tube. The tube was sealed with cellophane and the reaction stirred for 16 h at ambient temperature. The solvent was evaporated and the residue dissolved in DMSO containing 0.01% BHT to yield a 0.05 M solution. The solution was injected into an automated HPLC system for purification. The solvent of the product containing fraction was evaporated, the residue dissolved in DMSO, analyzed, and submitted for screening.

General Analysis and Purification Procedures

The crude reaction mixtures were analyzed by HPLC using Method 1. Prior to purification, all samples were filtered through Whatman® GF/F Unifilter (#7700-7210), commercially available from Whatman® of Clifton, N.J. USA. Purification of samples was performed by reverse phase HPLC using the method 3. Fractions were collected in 23 mL pre-tared tubes and centrifugal evaporated to dryness. Dried product was weighed and dissolved in DMSO. Products were then analyzed using Method 5 and submitted for screening.

Analytical LCMS Method 1 (Pre-Purification)

Column: Peeke Scientific® HI-Q C-18, 50×4.6 mm, commercially available from Peeke Scientific® of Redwood City, Calif., 5 μm, Eluent A: Water with 0.05% TFA, Eluent B: Acetonitrile with 0.05% TFA, Gradient: linear gradient of 0-100% B in 3.0 min, then 100% B for 0.5 min, then 100-0% B in 0.25 min, hold 100% A for 0.75 min, Flow: 2.25 mL/min, Column Temperature: 25° C., Injection Amount: 15 μl of a 286 μM crude solution in methanol/DMSO/water 90/5/5, UV Detection: 260 and 210 nm, Mass Spectrometry: APCI, positive mode, mass scan range 111.6-1000 amu.

Preparative LC Method 3 (Gilson)

Column: Peeke Scientific® HI-Q C18, 50 mm×20 mm, 5 μm, Eluent A: 0.05% TFA in Water, Eluent B: 0.05% TFA in Acetonitrile, Pre-inject Equilibration: 0.50 min, Post-inject Hold: 0.16 min, Gradient: 0-100% B in 2.55 minutes, then ramp 100% back to 0% in 0.09 min, Flow: 50.0 mL/min, Column Temp: Ambient, Injection Amount: 1200 μL of filtered crude reaction mixture in DMSO, Detection: UV at 210 nm or 260 nm.

Analytical LCMS Method 5 (Post-Purification)

Column: Peeke Scientific® HI-Q C-18, 50×4.6 mm, 5 μm, Eluent A: Water with 0.05% TFA, Eluent B: Acetonitrile with 0.05% TFA, Gradient: linear gradient of 0-100% B in 1.75 min, then 100% B for 0.35 min, then 100-50% B for 0.5 min, Flow: 3.00 mL/min, Column Temperature: 25° C., Injection Amount: 15 μl of a 300 μM solution in methanol/DMSO 99/1, UV Detection: 260 nm, Mass Spectrometry: APCI, positive mode, mass scan range 100-1000 amu, ELSD: gain=9, temp 40° C., nitrogen pressure 3.5 bar.

### Method F

### Example 33

### 4′-Cyano-biphenyl-4-sulfonic acid (6-methyl-pyridin-2-yl)-amide

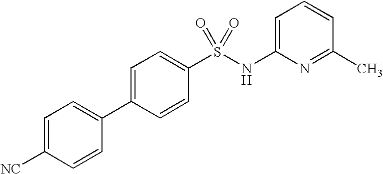

A solution of 4′-cyano-biphenyl-4-sulfonyl chloride (32.00 g, 115 mmol) and 2-amino-6-picoline (13.70 g, 127 mmol) in pyridine was stirred at room temperature for 18 h. The solvent was removed and the residue was poured into water (500 mL). The product was extracted with ethyl acetate (4×200 mL). The combine organic extracts were washed with brine and concentrated. Purification by flash silica chromatography on silica gel (40% ethyl acetate in hexanes→ethyl acetate) gave the title compound (28.80 g, 72%).

### Preparation of Sodium 4′-Cyanobiphenyl-4-sulfonate

(Modification of Himmelsbach, F.; Austel, V.; Pieper, H.; Eisert, W.; Mueller, T.; Weisenberger, J.; Linz, G.; Krueger, G. Eur. Pat Appl 1992, EP 483667 A2) Chlorosulfonic acid (116.5 mL, 1.744 mmol) was added to a solution of 4-biphenylcarbonitrile (156.2 g, 0.872 mol) in dichloromethane (3 L) at −14° C. while maintaining the reaction temperature below −10° C. The mixture was warmed to 10° C. over 1 h and maintained at 8-10° C. for 6 h. Triethylamine was added while maintaining the temperature below 12° C. The mixture was stirred for 15 min until all black/brown solids were dissolved and a while precipitate formed. Water (300 mL) was added, and the slurry was stirred for 10 min and concentrated. A solution of sodium hydroxide (2 L, 15%) was added, and the reaction mixture was concentrated until at least half of the volume was distilled. Concentrated hydrochloric acid (300 mL) was added until a pH of 7 was reached, and the final volume was adjusted to 2.2 L by the addition of water. A saturated solution of sodium chloride (2.2 L) was added, and the resulting mixture was stirred for 10 min. The solids were filtered and dried in a vacuum oven (80° C.) to afford 251.0 g of the product as a white to yellow solid. The product contains a substantial amount of sodium chloride.

### Preparation of 4′-Cyanobiphenyl-4-sulfonyl chloride

(Modification of Himmelsbach, F.; Austel, V.; Pieper, H.; Eisert, W.; Mueller, T.; Weisenberger, J.; Linz, G.; Krueger, G. Eur. Pat. Appl 1992, EP 483667 A2). A mixture of sodium 4′-cyanobiphenyl-4-sulfonate (251 g) and phosphorous oxychloride was refluxed for 16 h. The reaction mixture was poured into a large quantity of ice/water and the resulting slurry was extracted with dichloromethane (1×1.8 L). The organic extract was washed with brine, dried over magnesium sulfate, filtered, and concentrated to approximately 200 mL. Hexanes (200 mL) was added. The slurry was stirred for 30 min, filtered, washed with 1:1 dichloromethane/hexanes, and dried to give 82.1 g of product. The mother liquor was concentrated and further purified by flash chromatography on silica gel (40→70% dichloromethane/hexanes) to give an additional 16.2 g of white solid. ¹H NMR (300 MHz, CDCl₃) δ: 8.13-8.19 (m, 2 H), 7.80-7.86 (m, 4 H), 7.72-7.77 (m, 2 H). ¹³C NMR (75 MHz, CDCl₃) δ: 146.2, 144.2, 143.0, 133.2, 128.7, 128.4, 128.0, 118.5, 113.1.
Alternative General Method for Sulfonamide Formation

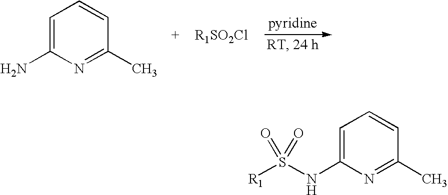

The sulfonyl chloride (104 μmol, 1.3 equiv 400 μL of a 0.26 M solution in anhydrous pyridine) and 2-amino-6-picoline (80 μmol, 1.0 equiv 400 μL of a 0.2 M solution in anhydrous pyridine) were placed into a test tube (75×10 mm, dried by heating at 110° C. for 16 h) equipped with a stir bar. The test tube was covered with Parafilm® and the reaction was stirred for 24 h at ambient temperature. The solvent was evaporated and the residue was dissolved in EtOAc (1 mL). After dissolution was completed or a fine suspension had formed, NaHCO₃ (0.5 mL of a sat aq. solution) was added. The reaction mixture was vortexed and the phases were separated by centrifugation. The organic layer was transferred into a new test tube (95×10 mm) and the aq. phase was extracted with EtOAc (2×0.8 mL). The organic phases were combined, the solvent was evaporated, and the residue was dissolved in DMSO (1.340 mL).

General Analysis and Purification Procedures

The crude reaction mixtures were analyzed by SFC using Method 2. Prior to purification, all samples were filtered through Whatman® GF/F Unifilter (#7700-7210). Purification of samples was performed by SFC using the method 4. Fractions were collected in 23 mL pre-tared tubes and centrifugal evaporated to dryness. Dried product was weighed and dissolved in DMSO. Products were then analyzed using Method 5 and submitted for screening.

Analytical SFC Method 2 (Pre-Purification)

Column: Zymor Pegasus, 150×4.6 mm i.d., 5 μm, Gradient: 5% methanol-modified CO2 ramped to 50% methanol @ 18%/min and held for 0.1 min, Flow rate: 5.6 mL/min, Column Temp.=50 C, Isobaric pressure: 140 bar, UV Detection=260 nm.

Preparative SFC Method 4

Column: Zymor Pegasus, 150×21.2 mm i.d., 5 μm semi-preparative column, Lot 2174, Column Temp: 35° C., Gradient: 5% methanol-modified CO₂ held for 0.1 minute, ramped to 60% methanol @ 10%/min and held for 1.0 minute, Flow Rate: 53 mL/min, Isobaric pressure: 140 bar, UV Detection: 260 nm.

Analytical LCMS Method 5 (Post-Purification)

Column: Peeke Scientific® HI-Q C-18, 50×4.6 mm, 5 μm, Eluent A: Water with 0.05% TFA, Eluent B: Acetonitrile with 0.05% TFA, Gradient: linear gradient of 0-100% B in 1.75 min, then 100% B for 0.35 min, then 100-50% B for 0.5 min, Flow: 3.00 mL/min, Column Temperature: 25° C., Injection Amount: 15 μl of a 300 μM solution in methanol/DMSO 99/1, UV Detection: 260 nm, Mass Spectrometry: APCI, positive mode, mass scan range 100-1000 amu, ELSD: gain=9, temp 40° C., nitrogen pressure 3.5 bar.

### Method G

### Example 110

### 4′-Cyano-biphenyl-4-sulfonic acid methyl-(6-methyl-pyridin-2-yl)-amide

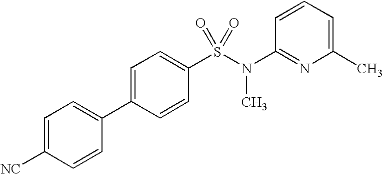

To a solution of N,6-dimethylpyridin-2-amine (0.15 g, 1.24 mmol) in THF (5 ml) was added NaHMDS (1.56 mL, 1.56 mmol) at R.T. After 15 min, 4′-cyanobiphenyl-4-sulfonyl chloride (0.28 g, 1.03 mmol) was added to the reaction mixture and stirred for 1 hour. The reaction mixture was diluted with ethyl acetate (30 mL) and washed with saturated aqueous sodium bicarbonate (2×30 mL). The collected organic layer was dried over anhydrous sodium sulfate, filtered, and concentrated. The resulting residue was purified with radial chromatography (2 mm silica plate, 2:1 hexanes/ethyl acetate) to yield a clear oil. The product was converted to a HCl salt by dissolving in 5 mL diethyl ether and adding 1 N HCl in diethyl ether dropwise. The solid was triturated with additional ether and dried on high vacuum to afford the product (0.11 g, 29.5%).

### Method H

### Example 111

### 4′-Cyano-biphenyl-4-sulfonic acid (6-isopropyl-pyridin-2-yl)-amide

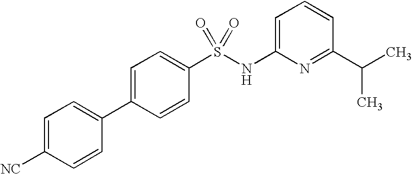

### Preparation of N-(6-Bromo-pyridin-2-yl)-2,2-dimethyl-propionamide

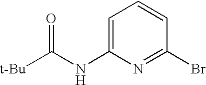

To an ice-cooled solution of 6-bromopyridin-2-amine (7.0 g, 40.5 mmol) in 60 mL of CH₂Cl₂ was added 2,2-dimethylpropanoyl chloride (5.23 mL, 42.48 mL) and diisopropylethylamine (13.6 mL, 82.9 mmol) sequentially. The solution was stirred for 1 h then diluted with 50 mL of diethyl ether. The mixture was washed with saturated aqueous sodium bicarbonate (2×50 mL). The organic layer was dried over Na₂SO₄, filtered, and concentrated. The residue was dissolved in ethyl acetate (10 mL) and hexane (20 mL) and allowed to stand for 3 h. The product was filtered, rinsed with 1:1 hexanes/ethyl acetate, and dried in vacuo to afford the title compound as a white solid (9.56 g, 93%). ¹H NMR (400 MHz, CD₃CN), δ: 8.22 (d, J=8.4 Hz, 1 H), 7.99 (bs, 1 H), 7.55 (t, J=8.1 Hz, 1 H), 7.22 (d, J=7.3 Hz, 1 h), 1.31 (s, 9 H); LCMS (ESI): m/z: 258.0.

### Preparation of N-(6-isopropyl-pyridin-2-yl)-2,2-dimethyl-propionamide

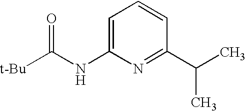

Cul (7.40 g, 38.8 mmmol) was added to a solution of N-(6-bromopyridin-2-yl)-2,2-dimethylpropanamide (5.0 g, 19.4 mmol) in THF (100 mL) at −78° C. After 0.5 hours, isopropylmagnesium chloride (48.5 mL, 1M in THF) was added dropwise at −78° C., and the resulting solution was warmed to 25° C. for 2 hours. The reaction was quenched with saturated aqueous ammonium chloride (50 mL) then diluted with ethyl acetate (100 mL). The solids were removed by filtration. The solution was washed sequentially with saturated aqueous ammonium chloride (2×50 mL) and saturated aqueous sodium bicarbonate (2×50 mL). The organic layer was dried over Na₂SO₄, filtered, and concentrated. Purification by flash column chromatography (2:1 hexane/ethyl acetate) afforded the title product as an amber oil (2.60 g, 60.4%). ¹H NMR (400 MHz, CD₃CN), δ: 8.04 (d, J=7.8 Hz, 1 H), 7.97 (bs, 1 H), 7.63 (t, J=7.8 Hz, 1 H), 6.90 (d, J=7.5 Hz, 1 H), 2.95-2.88 (m, 1 H), 1.34 (s, 9 H), 1.28 (d, J=7.1 Hz, 6 H); LCMS (ESI): m/z: 221.2.

### Preparation of 6-isopropyl-pyridin-2-ylamine

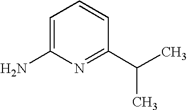

To a solution of N-(6-isopropylpyridin-2-yl)-2,2-dimethylpropanlamide (2.0 g, 9.08 mmol) in dioxane (5 mL) was added HCl (9N, 10 mL). The mixture was stirred for 18 hours at 80° C. After cooling to 25° C., the pH of the reaction mixture was adjusted with NaOH to achieve pH 9. The solution was diluted with ethyl acetate (120 mL) and washed with saturated aqueous sodium bicarbonate (2×30 mL). Next, the organic layer was azeotroped with toluene (10 mL) to afford 6-isopropylpyridin-2-amine as clear oil (0.68 g, 55%). ¹H NMR(400 MHz, CD₃CN), δ: 7.36 (t, J=7.8 Hz, 1 H), 6.64 (d, J=8.7, 1 H), 6.32 (d, J=8.1 Hz, 1 H), 1.25 (d, J=4.5 Hz, 9 H); LCMS (ESI): m/z: 137.2.

### 4′-Cyano-biphenyl-4-sulfonic acid (6-isopropyl-pyridin-2-yl)-amide

Made following the procedure described for the preparation of 4′-cyano-biphenyl4-sulfonic acid (6-methyl-pyridin-2-yl)-amide but substituting 6-isopropyl-pyridin-2-ylamine and making non-critical variations.

### Method I

### Example 112: 4′-Cyano-biphenyl-4-sulfonic acid (6-cyclopropyl-pyridin-2-yl)-amide

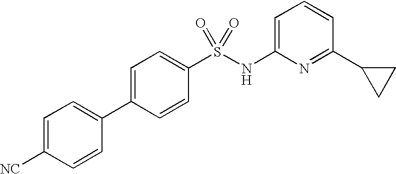

### Preparation of N-(6-Cyclopropyl-pyridin-2-yl)-2,2-dimethyl-propionamide

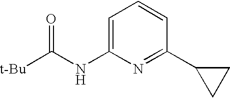

To a solution of N-(6-bromopyridin-2-yl)-2,2-dimethylpropanamide (4.20 g, 16.3 mmol), cyclopropylboronic acid (1.82 g, 21.8 mmol), Pd(OAc)₂ (0.18 g, 0.82 mmol) and PCy₃ (0.38 g, 1.62 mmol) in toluene (20 mL) was added K₃PO₄ (12.8 g, 60.3 mmol) and water (1 mL). The mixture was stirred at 95° C. for 12 h, then cooled to 25° C. The reaction mixture was diluted with Et₂O (30 mL) and washed with saturated aqueous sodium bicarbonate. The organic layer was dried over Na₂SO₄, filtered, and concentrated to give a clear oil. The residue was purified by flash column chromatography (5:1 hexanes/Et₂O) to give the title product as a clear oil (2.25 g, 63.3%). ¹H NMR (400 MHz, CDCl₃), δ: 7.98 (d, J=8.3, 1 H), 7.88 (bs, 1 H), 7.53 (t, J=7.8 Hz, 1 H), 6.85 (d, J=7.5 Hz, 1 H), 1.98-1.91 (m, 1 H)m 1.32 (s, 9 H), 0.94 (d, J=6.6 Hz, 4 H); LCMS (ESI): 219.2.

### Preparation of 6-Cyclopropyl-pyridin-2-ylamine

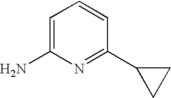

Made by following the procedure described for the preparation of 6-isopropyl-pyridin-2-ylamine but substituting N-(6-cyclopropyl-pyridin-2-yl)-2,2-dimethyl-propionamide and making non-critical variations. ¹H NMR(400 MHz, CDCl₃), δ: 7.70 (t, J=7.8, 1 H), 6.85 ((t, J=7.4, 1 H), 6.65 (d, J=7.5 Hz, 1 H), 4.79 (bs, 2 H); LCMS (ESI): m/z: 135.2.

### 4′-Cyano-biphenyl-4-sulfonic acid (6-cyclopropyl-pyridin-2-yl)-amide

Made by following the procedure described for the preparation of 4′-cyano-biphenyl-4-sulfonic acid (6-methyl-pyridin-2-yl)-amide but substituting 6-cyclopropyl-pyridin-2-ylamine and making non-critical variations.

### Method J

### Example 113

### 4′-Cyano-biphenyl-4-sulfonic acid (6-amino-4-methyl-pyridin-2-yl)-amide

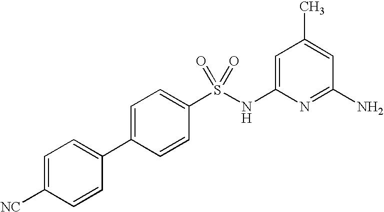

To a solution of 4-methylpyridine-2,6-diamine (J. Org. Chem. 2001, 61, 6513)(102 mg, 0.825 mmol) in THF (6 mL) was added diispropylethylamine (287 μL, 1.65 mmol) followed by 4-(dimethylamino)pyridine (5 mg, 0.04 mmol). To the resulting solution was added 4′-cyanobiphenyl-4-sulfonyl chloride in CH₂Cl₂ (3 mL). The heterogeneous mixture was stirred at R.T. overnight. By morning all solids had dissolved and the solution was concentrated in vacuo. The residue was dissolved in MeOH/CH₂Cl₂ and to the solution was added DOWEX® 50WX2A-400 ion exchange resin, commercially available from DOW Company of Midland, Mich. USA, (2 wt equiv) and the mixture was stirred at R.T. for 1 hour. The mixture was filtered and the resin was washed with MeOH and CH₂Cl₂. The resin was then cleaved by washing with 3.5 N methanolic ammonia and the mother liquor was concentrated in vacuo. To the residue was added MeOH, and the solids were filtered to afford the title compound (50 mg, 25%).

### Method K

### Example 114

### 3-Chloro-N-[6-(2-hydroxy-ethyl)-pyridin-2-yl]-2-methyl-benzenesulfonamide

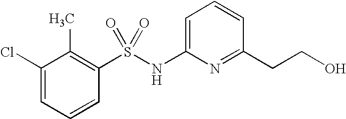

Borane-tetrahydrofuran complex (0.924 mL, 0.924 mmol, 3.0 equiv, 1.0 M tetrahydrofuran solution) was added to an ice-cooled solution of [6-(3-chloro-2-methyl-benzenesulfonylamino)-pyridin-2-yl]-acetic acid (105 mg, 0.308 mmol, 1 equiv) in tetrahydrofuran. After 1 h, the reaction mixture was warmed to 24° C. for 17.5 h. Aqueous hydrochloric acid (3 mL, 5% wt) was added, and the resulting solution was stirred vigorously. After 30 min, saturated aqueous sodium bicarbonate solution (8 mL) was added, and the mixture was extracted with dichloromethane (3×15 mL). The collected organic extracts were dried over anhydrous sodium sulfate, filtered, and concentrated. Purification by high performance flash chromatography (0→5% methanol in dichloromethane) yielded product (45.5 mg, 45%).

### Method L

### Example 115

### 5-Chloro-3-methyl-benzo[b]thiophene-2-sulfonic acid [6-(2-hydroxy-ethyl)-pyridin-2-yl]-amide

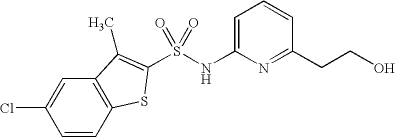

Lithium aluminum hydride (0.015 g, 0.310 mmol, 1.3 equiv) was added in one portion to an ice-cooled solution of [6-(5-Chloro-3-methyl-benzo[b]thiophene-2-sulfonylamino)-pyridin-2-yl]-acetic acid ethyl ester (0.100 g, 0.235 mmol, 1 equiv) in tetrahydrofuran (4 mL). After 5 min, the reaction mixture was warmed to 24° C. for 16 h. The reaction mixture was cooled to 0° C., and excess lithium aluminum hydride was quenched with saturated aqueous ammonium chloride solution (10 mL). The resulting solution was warmed to 24° C. and stirred for an additional 30 min. The reaction mixture was filtered through a plug of Celite®, and the resulting filtrate was extracted with dichloromethane (60 mL). The organic extract was dried over anhydrous sodium sulfate, filtered, and concentrated. Purification of the residue by high performance flash chromatography (0→1% methanol in dichloromethane) yielded product (0.0421 g, 47%).

### Method M

### Example 118

### 2-(4-Cyano-phenyl)-4-methyl-thiazole-5-sulfonic acid (6-methyl-pyridin-2-yl)-amide

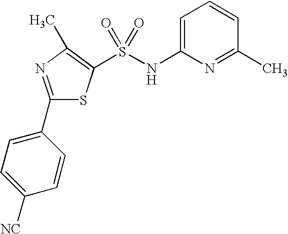

### Preparation of N-[4-Methyl-5-(6-methyl-pyridin-2-ylsulfamoyl)4hiazol-2-yl]-acetamide

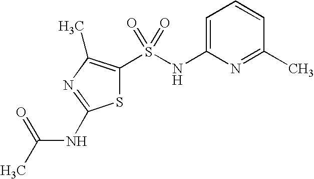

Made by following the procedure described for the preparation of 4′-cyano-biphenyl-4-sulfonic acid (6-methyl-pyridin-2-yl)-amide but substituting 2-acetamido-4-methyl-5-thiazole sulfonyl chloride and making non-critical variations. ¹H NMR (400 MHz, CDCl₃), δ: 7.56 (dd, J=8.7, 7.2 Hz, 1 H), 7.10 (d, J=8.6 Hz, 1 H), 6.58 (d, J=7.3 Hz, 1 H), 2.53 (s, 3 H), 2.47 (s, 3 H), 2.24 (s, 3 H); MS (ESI) for C₁₂H₁₅N₄O₃S₂ m/z: 327.0.

### Preparation of 2-Amino-4-methyl-thiazole-5-sulfonic acid (6-methyl-pyridin-2-yl)-amide

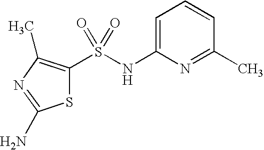

A solution of N-[4-methyl-5-(6-methyl-pyridin-2-ylsulfamoyl)-thiazol-2-yl]-acetamide (2.15 g, 6.58 mmol, 1 equiv) and aqueous hydrochloric acid (1.6 mL, 12 M) in ethanol (30 mL) was refluxed overnight. Upon cooling to 24° C., the reaction mixture was concentrated in vacuo (25 mm Hg). The resulting solid was dissolved in water (10 mL). The solution was neutralized with saturated aqueous sodium bicarbonate until pH=7. The resulting solid was collected by filtration. Lyophilization of the solid provided an off-white solid (1.67 g, 89%). ¹H NMR (400 MHz, DMSO-d₆), δ: 7.64 (t, J=8.0 Hz, 1 H), 7.44 (s, 2 H), 6.93 (m, 1 H), 6.70 (m, 1 H), 2.32 (s, 3 H), 2.27 (s, 3 H); MS (ESI) for C₁₀H₁₃N₄O₂S₂ m/z: 285.1.

### Preparation of 2-Bromo-4-methyl-thiazole-5-sulfonic acid (6-methyl-pyridin-2-yl)-amide

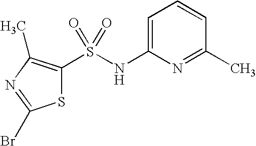

To a suspension of 2-amino4-methyl-thiazole-5-sulfonic acid (6-methyl-pyridin-2-yl)-amide (0.200 g, 0.703 mmol, 1 equiv) and copper (II) bromide (0.098 g, 0.68 mmol, 0.62 equiv) in acetonitrile (6 mL) at 65° C. was added tert-butyl nitrite (0.128 mL, 1.08 mmol, 1.5 equiv). The reaction mixture changed from green to red and gas evolution was observed. After 10 min when gas evolution ceased, the reaction mixture was cooled to 24° C. and diluted with ethyl acetate (60 mL). The resulting mixture was washed with saturated aqueous sodium chloride (2×30 mL). The collect organic was dried over anhydrous sodium sulfate, filtered, and concentrated. Purification by high performance flash chromatography (0→2% methanol in dichloromethane) provided product (0.156 g, 64%). ¹H NMR (400 MHz, CDCl₃), δ: 7.61 (dd, J=8.8, 7.1 Hz, 1 H), 7.00 (d, J=8.8 Hz, 1 H), 6.58 (d, J=7.3 Hz, 1 H), 2.65 (s, 3 H), 2.49 (s, 3 H); MS (ESI) for C₁₀H₁₁BrN₃O₂S₂ m/z: 349.9.

### 2-(4-Cyano-phenyl)-4-methyl-thiazole-5-sulfonic acid (6-methyl-pyridin-2-yl)-amide

A solution of 2-bromo-4-methyl-thiazole-5-sulfonic acid (6-methyl-pyridin-2-yl)-amide (0.080 g, 0.23 mmol, 1 equiv), 4-cyanophenylboronic acid (0.034 g, 0.23 mmol, 1.0 equiv), and cesium carbonate (0.225 g, 0.690 mmol, 3.00 equiv) in 2:1 dimethoxyethane/water (1.5 mL) was purged with nitrogen for 15 min. Dichloro[1,1′-bis(diphenylphosphine)ferrocene] palladium (II) chloride (0.008 g, 0.009 mmol, 0.04 equiv) was then added, and the resulting mixture was purged with nitrogen for another 15 minutes. The reaction mixture was heated to 80° C. for 1 h. After cooling to 24° C., the resulting solution was diluted with ethyl acetate (40 mL) and washed with saturated aqueous sodium chloride (2×30 mL). The collected organic was dried over anhydrous sodium sulfate, filtered, and concentrated. Purification by high performance flash chromatography (0→1% methanol in dichloromethane) provided the titled compound (62 mg, 73%).

### Method N

### Preparation of 4-Bromo-N-(6-methyl-pyridin-2-yl)-benzenesulfonamide

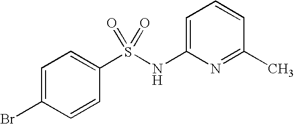

Made by following the procedure described for the preparation of 4′-cyano-biphenyl-4-sulfonic acid (6-methyl-pyridin-2-yl)-amide but substituting 4-bromobenzenesulfonyl chloride and making non-critical variations. 1H NMR (400 MHz, CDCl₃), δ ppm 7.61-7.68 (m, 2 H) 7.40-7.46 (m, 2 H) 7.36 (dd, J=8.6, 7.3 Hz, 1 H) 6.77-6.83 (d, J=8.8 Hz, 1 H), 6.42 (d, J=7.1 Hz, 1 H) 2.28 (s, 3 H).

### Preparation of 4-Bromo-2-methyl-N-(6-methyl-pyridin-2-yl)-benzenesulfonamide

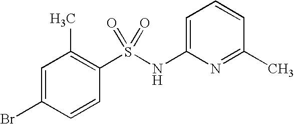

Made by following the procedure described for the preparation of 4′-cyano-biphenyl-4-sulfonic acid (6-methyl-pyridin-2-yl)-amide but substituting 4-bromo-2-methylbenzene-1-sulfonyl chloride (commercially available from ASDI, Inc. of Newark, Del. USA) and making non-critical variations. APCI⁺342 [M+H]⁺100%.

### Preparation of 4-Bromo-3-methyl-N-(6-methyl-pyridin-2-yl)-benzenesulfonamide

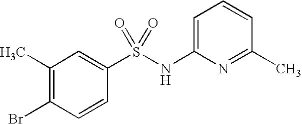

Made by following the procedure described for the preparation of 4′-cyano-biphenyl-4-sulfonic acid (6-methyl-pyridin-2-yl)-amide but substituting 4-bromo-3-methylbenzene-1-sulfonyl chloride (available from Lancaster) and making non-critical variations. APCI⁺342 [M+H]⁺100%.

General Method for Microwave Assisted Suzuki-Miyaura Cross-Coupling

This protocol discloses a procedure for the synthesis of biaryls through a Suzuki-Miyaura cross-coupling of an 4-bromobenzenesulfonamide (Reactant A) and an aryl boronic-acid (Reactant B). 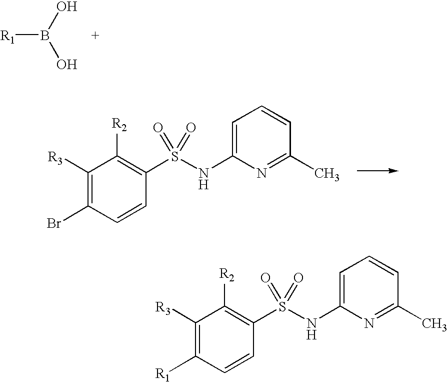
Preferred Conditions:

In a glove box, the following was added to a 2.0 mL Personal Chemistry Microwave reaction tube:
(1) one triangular stir bar,
(2) 4-Bromobenzenesulfonamide (Reactant A, 320 μL, 80 μmol, 1.0 equiv, 0.25 M in anhydrous DMF),
(3) the appropriate aromatic boronic acid (Reactant B, 320 μL, 80 μmol, 1.0 equiv, 0.25 M in anhydrous DMF),
(4) the catalyst Pd(PPh₃)₄ (320 μL, 4 μmol, 0.05 equiv, 0.0125 M in anhydrous THF), and
(5) K₂CO₃ (100 μL, 200 μmol, 2.5 equiv, 2 M in degassed DI water).
(6) The microwave tube was sealed with a septum cap.

Outside the glove box, the reaction mixtures were heated in a Personal Chemistry Microwave Synthesizer (SmithCreator™) for 15 min at 130° C. (energy-control setting for a high absorbing sample). The septum caps were removed and the reaction mixture was transferred into a 13×100 mm test tube while leaving any solid material behind. The microwave tubes were washed with DMF (1 mL) and the DMF was added to the receiving test tube.

Next, the solvent was evaporated (SpeedVac, vaccum, medium heating, 16 h). EtOAc (1 mL) and water (1.0 mL) were added and the mixture was vortexed at ambient temperature until the residue had dissolved (Note: Some of the palladium in the reaction mixture will form a small amount of a black material that will not dissolve). The test tubes were centrifuged until the phases had separated (some of the black palladium material will settle at the organic/aqueous interface). The organic layer was transferred into a new test tube (13×100 mm). The aq. layer was extracted with EtOAc (2×1 mL) and the extracts were added to the test tube with the organic layer. The combined organic phase was washed with water (1 mL) followed by brine (1 mL). The solvent was evaporated and the residue dissolved in DMSO. Purification was peformed by reverse phase preparative HPLC.

General Analysis and Purification Procedures

The crude reaction mixtures were analyzed by HPLC using Method 1. Prior to purification, all samples were filtered through Whatman® GF/F Unifilter (#7700-7210). Purification of samples was performed by reverse phase HPLC using the method 3. Fractions were collected in 23 mL pre-tared tubes and centrifugal evaporated to dryness. Dried product was weighed and dissolved in DMSO. Products were then analyzed using Method 5 and submitted for screening.

Analytical LCMS Method 1 (Pre-Purification)

Column: Peeke Scientific® HI-Q C-18, 50×4.6 mm, 5 μm, Eluent A: Water with 0.05% TFA, Eluent B: Acetonitrile with 0.05% TFA, Gradient: linear gradient of 0-100% B in 3.0 min, then 100% B for 0.5 min, then 100-0% B in 0.25 min, hold 100% A for 0.75 min, Flow: 2.25 ml/min, Column Temperature: 25° C., Injection Amount: 15 μl of a 286 μM crude solution in methanol/DMSO/water 90/5/5, UV Detection: 260 and 210 nm, Mass Spectrometry: APCI, positive mode, mass scan range 111.6-1000 amu.

Preparative LC Method 3 (Gilson)

Column: Peeke Scientific® HI-Q C18, 50×20 mm, 5 μm, Eluent A: 0.05% TFA in Water, Eluent B: 0.05% TFA in Acetonitrile, Pre-inject Equilibration: 0.50 min, Post-inject Hold: 0.16 min, Gradient: 0-100% B in 2.55 minutes, then ramp 100% back to 0% in 0.09 min, Flow: 50.0 mumin, Column Temp: Ambient, Injection Amount: 1200 μL of filtered crude reaction mixture in DMSO, Detection: UV at 210 nm or 260 nm.

Analytical LCMS Method 5 (Post-Purification)

Column: Peeke Scientific® HI-Q C-18, 50×4.6 mm, 5 μm, Eluent A: Water with 0.05% TFA, Eluent B: Acetonitrile with 0.05% TFA, Gradient: linear gradient of 0-100% B in 1.75 min, then 100% B for 0.35 min, then 100-50% B for 0.5 min, Flow: 3.00 mL/min, Column Temperature: 25° C., Injection Amount: 15 μl of a 300 μM solution in methanol/DMSO 99/1, UV Detection: 260 nm, Mass Spectrometry: APCI, positive mode, mass scan range 100-1000 amu, ELSD: gain=9, temperature 40° C., nitrogen pressure 3.5 bar.

### Method O

### Example 249

### 4′-Chloro-biphenyl-4-sulfonic acid (6-methyl-pyridin-2-yl)-amide

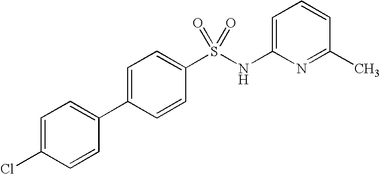

To a mixture of 4-Bromo-N-(6-methyl-pyridin-2-yl)-benzenesulfonamide (160 mg, 0.489 mmol) and 4-chlorophenylboronic acid (76.5 mg, 0.489 mmol) in DMF (2 mL) was added aqueous Na₂CO₃ (2.0 M, 0.625 mL; 1.25 mmol) followed by Pd(PPh₃)₄ (28 mg, 0.0245 mmol). The resulting mixture was heated at 130° C. for 15 min in microwave oven. The mixture was cooled and partitioned between ethyl acetate and water. The organic layer was dried over sodium sulfate, filtered and concentrated. The residue was purified by silica gel chromatography (50% EtOAc/Hexane) to yield the title compound as a yellow solid (130 mg, 74%).

### Method P

### Example 259

### N-(6-Methyl-pyridin-2-yl)-4-pyridin-2-yl-benzenesulfonamide trifuoroacetate

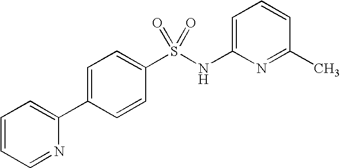

A mixture of 4-Bromo-N-(6-methyl-pyridin-2-yl)-benzenesulfonamide (117 mg, 0.358 mmol), 2-pyridyltributyltin (197 mg, 0.536 mol) and Pd (PPh₃)₂Cl₂ (13 mg, 0.018 mmol) in DMF (2 mL) was heated in a microwave oven for 1 h. DMF was removed under vacuum. The residue was purified by reverse phase preparative HPLC to yield the title compound as white solid (42 mg, 0.129 mmol; 36%).

### Method Q

### Example 262

### 4′-(6-Methyl-pyridin-2-ylsulfamoyl)-biphenyl-4-carboxylic acid amide

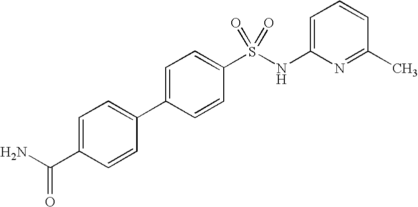

To a solution of 4′-cyano-biphenyl-4-sulfonic acid (6-methyl-pyridin-2-yl)-amide (144 mg, 0.286 mmol) in 30% H₂O₂ (1 mL) and EtOH (1 mL) was added 4N NaOH (0.2 mL). The mixture become clear. After 12 h, the mixture was partitioned between EtOAc and H₂O. The organic layer was washed with brine, dried over sodium sulphate and concentrated. The residue was chromatographed over silica gel (60% EtOAc/hexane) to give the title compound as a white solid.

### Method R

### Example 263

### 4′-(2-Amino-ethoxy)-biphenyl-4-sulfonic acid (6-methyl-pyridin-2-yl)-amide

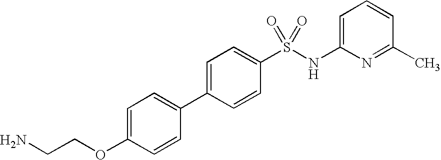

To a yellow solution of 4-hydroxy-biphenyl-4-sulfonic acid (6-methyl-pyridin-2-yl)-amide (129 mg, 0.378 mmol), N-hydroxyethylphthaliamide (80 mg, 0.416 mmol), triphenylphosphine (119 mg, 0.454 mmol) in THF (3 mL) was added DEAD (72 μL, 0.454 mmol). After stirring overnight, the mixture was concentrated. The residue was chromatographed on silica gel (40-70% EtOAc/hexane) to give the ether intermediate (152 mg, 79%). To a solution of the above ether intermediate (152 mg, 0.3 mmol) in MeOH (3 mL) was added hydrazine (74 μL, 1.5 mmol). The mixture was stirred at R.T. for 2 h and concentrated to give a residue, which was purified by preparative HPLC to give the final product as a white solid (60 mg, 52%).

### Method S

### Example 264

### N-(6-Methyl-pyridin-2-yl)-4-oxazol-5-yl-benzenesulfonamide

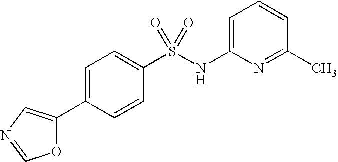

### Preparation of 4-Formyl-N-(6-methyl-pyridin-2-yl)-benzenesulfonamide

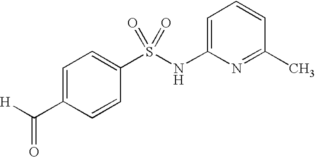

Made by following the procedure described for the preparation of 4′-cyano-biphenyl-4-sulfonic acid (6-methyl-pyridin-2-yl)-amide but substituting 4-formylbenzensulfonyl chloride.

### N-(6-Methyl-pyridin-2-yl)-4-oxazol-5-yl-benzenesulfonamide

A solution of sulfonamide from step 1 (449 mg, 1.63 mmol), TsCH₂NC (349 mg, 1.79 mmol) and K₂CO₃ (450 mg, 3.25 mmol) in MeOH (5 mL) was refluxed for 12 h. The mixture was cooled to R.T. and partitioned between EtOAc and water. The organic layer was dried over sodium sulfate and concentrated to give a residue, which was purified by flash column chromatography (60% EtOAc/hexanes) to give the title compound as a white solid (301 mg, 58% yield). ¹H NMR (400 MHz, CDCl₃), δ: 8.21 (s, 1 H), 7.90 (d, J=8.3 Hz, 1 H), 7.62 (d, J=8.3 Hz, 1 H), 7.56 (s, 1 H), 7.54 (m, 1 H), 7.04 (m, 1 H), 6.56 (m, 1 H), 2.30 (s, 3 H). Anal. Calcd for C₁₅H₁₃N₃O₃S: C, 57.13; H, 4.16, N, 13.33; Found: C, 57.31; H, 4.22; N, 12.92.

### Method T

### Example 265

### 4′-Cyano-biphenyl-4-sulfonic acid (2-dimethylamino-ethyl)-(6-methyl-pyridin-2-yl)-amide

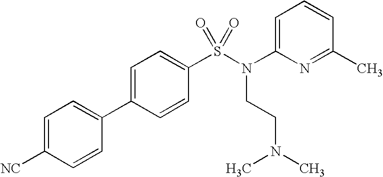

2-(Dimethylamino)ethyl chloride hydrochloride (70 mg, 0.49 mmol, 1.8 equiv) was added to a solution of 4′-cyano-biphenyl-4-sulfonic acid (6-methyl-pyridin-2-yl)-amide (93.1 mg, 0.266 mmol, 1 equiv), potassium carbonate (184 mg, 1.33 mmol, 5.00 equiv) in dimethylformamide (2.5 mL) at 24° C. The heterogenous solution was heated to 50° C. for 22 h. Upon cooling to 24° C., the reaction mixture was concentrated in vacuo (<1 mm Hg). The resulting residue was diluted with saturated aqueous sodium chloride (5 mL), saturated aqueous sodium bicarbonate (5 mL), and ethyl acetate (5 mL). The organic phase was separated, and the resulting aqueous solution was extracted with ethyl acetate (2×5 mL). The collected organic was dried over anhydrous sodium sulfate, filtered, and concentrated. Purification by high performance flash chromatography (0→5% methanol/dichloromethane+0.1% ammonium hydroxide) yielded alkylation product, which was converted to the hydrochloride salt by treatment with a methanolic hydrogen chloride solution (96.6 mg, 76%).

### Method U

### Example 266

### 4′-Cyano-biphenyl-4-sulfonic acid (2-hydroxy-ethyl)-(6-methyl-pyridin-2-yl)-amide

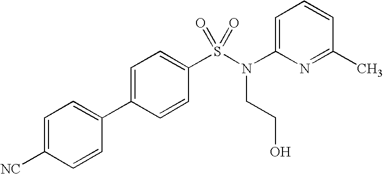

### Preparation of 4′-Cyano-biphenyl-4-sulfonic acid [2-(tert-butyl-dimethyl-silanyloxy)-ethyl]-(6-methyl-pyridin-2-yl)-amide

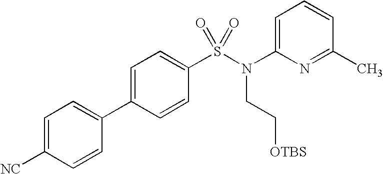

(2-Bromoethoxy)-tert-butyldimethylsilane (91 μL, 0.42 mmol, 1.5 equiv) was added to a solution of 4′-cyano-biphenyl-4-sulfonic acid (6-methyl-pyridin-2-yl)-amide (99.1 mg, 0.284 mmol, 1 equiv) and potassium carbonate (202 mg, 1.46 mmol, 5.2 equiv) in dimethylformamide (2.5 mL) at 24° C. The reaction mixture was maintained at 24° C. for 4.7 h before warming to 70° C. for 15.7 h. The reaction mixture was cooled to 24° C and concentrated in vacuo (<1 mm Hg). The resulting residue was diluted with ethyl acetate (5 mL), saturated aqueous sodium chloride (3 mL), and saturated aqueous sodium bicarbonate (3 mL). The organic layer was separated, and the resulting aqueous layer was extracted with ethyl acetate (2×5 mL). The collected organic extracts were dried over anhydrous sodium sulfate, filtered, and concentrated. Purification by high performance flash chromatography (12→50% ethyl acetate in hexanes) provided product (85.3 mg, 59%). ¹H NMR (400 MHz, CDCl₃), δ: 7.57-7.83 (m, 9 H), 7.40 (d, J=8.1 Hz, 1 H), 6.99 (d, J=7.6 Hz, 1 H), 4.00 (t, J=6.2 Hz, 2 H), 3.78 (t, J=6.2 Hz, 2 H), 2.41 (s, 3 H), 0.78 (s, 9 H), −0.03 (s, 6 H).

### 4′-Cyano-biphenyl-4-sulfonic acid (2-hydroxy-ethyl)-(6-methyl-pyridin-2-yl)-amide

Tetrabutylammonium flouride (371 mL, 0.371 mmol, 2.0 equiv, 1.0 M in tetrahydrofuran) was added dropwise to an ice-cooled solution of 4′-Cyano-biphenyl-4-sulfonic acid [2-(tert-butyl-dimethyl-silanyloxy)-ethyl]-(6-methyl-pyridin-2-yl)-amide (85.3 mg, 0.186 mmol, 1 equiv) in tetrahydrofuran (3 mL). After 50 min, saturated aqueous sodium chloride was added to the reaction mixture, and the resulting solution was extracted with ethyl acetate (3×5 mL). The collected organic extracts were dried over sodium sulfate, filtered, and concentrated. Purification by high performance flash chromatography (13% ethyl acetate in hexanes ethyl acetate) provided product which was converted to the hydrochloride salt by treatment with a methanolic hydrogen chloride solution (58 mg, 76%).

### Method V

### Example 267

### 6-(4-Cyano-phenyl)-pyridine-3-sulfonic acid (6-methyl-pyridin-2-yl)-amide

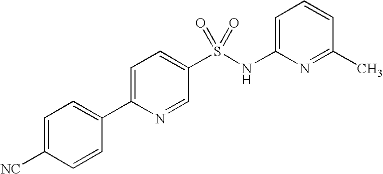

### Preparation of 6-Chloro-pyridine-3-sulfonic acid (6-methyl-pyridin-2-yl)-amide

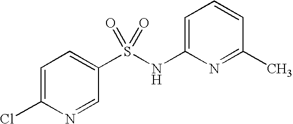

Made by following the procedure described for the preparation of 4′-cyano-biphenyl-4-sulfonic acid (6-methyl-pyridin-2-yl)-amide but substituting 6-chloro-3-pyridylsulfonyl chloride (Naegeli, C.; Kundig, W.; Brandenburger, H. Helv. Chem. Acta. 1939, 21, 1746) and making non-critical variations. APCI⁺284 [M+H]⁺100%.

### 6-(4-Cyano-phenyl)-pyridine-3-sulfonic acid (6-methyl-pyridin-2-yl)-amide

A solution of 6-chloro-pyridine-3-sulfonic acid (6-methyl-pyridin-2-yl)-amide (188 mg, 0.573 mmol), 4-cyanoboronic acid (88 mg, 0.602 mmol), Pd(PPh₃)₄ (33 mg, 0.03 mmol), aqueous Na₂CO₃ (0.72 mL, 1.43 mmol) in DMF (3 mL) was heated in microwave for 30 min. The black mixture was partitioned between EtOAc and water. The organic layer was then washed with brine, dried over Na₂SO₄ and concentrated to give an oil, which was chromatographed on silica gel to give title compound (86.3 mg, 43%) as a yellow solid.

### Method W

### Example 269

### N-(6-methylpyridin-2-yl)-6-piperidin-1-ylpyridine-3-sulfonamide

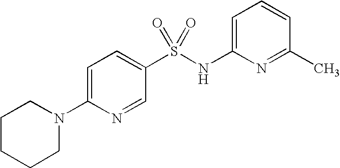

A mixture of 6-chloro-pyridine-3-sulfonic acid (6-methyl-pyridin-2-yl)-amide (233 mg, 0.823 mmol) and piperidine (4.17 mmol) in dioxane (5 mL) was heated at 100° C. in a Personal Chemistry Microwave oven for 30 min. The mixture was cooled and partitioned between EtOAc and water. The organic layer was dried over sodium sulfate, filtered, and concentrated. Purification by flash column chromatography (50 to 70% EtOAc/Hexanes) furnished the title compound as a brown solid (177 mg, 65%).

### Method X

### Example 270

### 4′-Cyano-3′-methoxy-biphenyl4-sulfonic acid (6-methyl-pyridin-2-yl)-amide

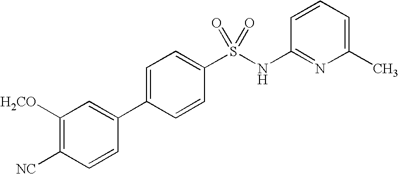

### Preparation of N-(6-Methyl-pyridin-2-yl)-4-(4,4,5,5-tetramethyl-[1,3,2]dioxaborolan-2-yl)-benzenesulfonamide

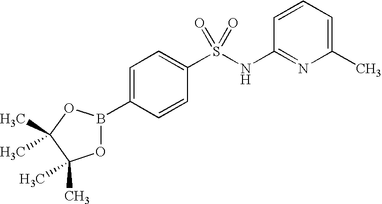

A mixture of 4-bromo-N-(6-methyl-pyridin-2-yl)-benzenesulfonamide (13.7 g, 41.9 mmol), bis(pinacolato)diboron (10.7 g, 41.9 mmol), KOAc (14 g, 143 mmol) and Pd(dppf)Cl₂ (1.7 g, 2.1 mmol) in DMSO (100 mL) was heated at 100° C. for 12 h. The mixture was cooled to room temperature, partitioned between EtOAc and water and filtered through Celite®. The organic layer was dried and concentrated. Purification by flash column chromatography (50% EtOAc/hexanes) furnished the boronate as a solid (15.5 g, 98%). ¹H NMR (400 MHz, CDCl₃) δ ppm 7.85-7.94 (m, 4 H) 7.44-7.51 (m, 1 H) 7.02 (d, J=8.6 Hz, 1 H) 6.60 (d, J=7.3 Hz, 1 H) 2.41 (s, 3 H) 1.31-1.35 (s,12 H).

### 4′-Cyano-3′-methoxy-biphenyl-4-sulfonic acid (6-methyl-pyridin-2-yl)-amide

Made by following the procedure described for the preparation of 4′chlorobiphenyl-4-sulfonic acid (6-methyl-pyridin-2-yl)-amide but substituting N-(6-methyl-pyridin-2-yl)-4-(4,4,5,5-tetramethyl-[1,3,2]dioxaborolan-2-yl)-benzenesulfonamide and 4-bromo-2-methoxybenzonitrile and making non-critical variations.

### Method Y

### Example 276

### 4′-Cyano-3-methoxy-biphenyl-4-sulfonic acid (6-methyl-pyridin-2-yl)-amide

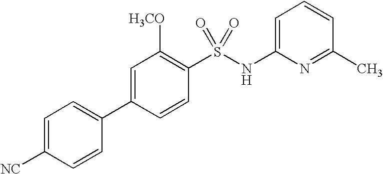

### Preparation of 4-Bromo-2-methoxy-N-(6-methyl-pyridin-2-yl)-benzenesulfonamide

To a solution of 1-bromo-3-methoxybenzene (3.1 g, 16.6 mmol) in CH₂Cl₂ at 0° C. was added ClSO₃H (3.3 mL, 48 mmol). The mixture was warmed to R.T. and stirred for 2 h. The mixture was poured into ice and water and extracted with CH₂Cl₂ (3×30 mL). The organic layer was dried over Na₂SO₄, filtered, and concentrated to give a mixture of sulfonyl chlorides as an oil, which was used for the next reaction without purification.

The above sulfonyl chloride was dissolved in pyridine (50 mL) and 2-methyl-6-aminopyridine (1.7 g, 16 mmol) was added. The mixture was stirred overnight at R.T. The mixture was partitioned between EtOAc and water. The organic layer was dried and concentrated to the mixture of sulfonamides (3 to 1 by LCMS). The residue was purified by flash column chromatography to give the desired isomer as a white solid (0.87 g, 15% for two steps).

### 4′-Cyano-3-methoxy-biphenyl-4-sulfonic acid (6-methyl-pyridin-2-yl)-amide

Made by following the procedure described for the preparation of 4′-chlorobiphenyl-4-sulfonic acid (6-methyl-pyridin-2-yl)-amide but substituting 4-bromo-2-methoxy-N-(6-methyl-pyridin-2-yl)-benzenesulfonamide and 4-cyanophenylboronic acid and making non-critical variations.

### Method Z

### Example 277

### 4′-Cyano-3-methyl-biphenyl-4-sulfonic acid (6-methyl-pyridin-2-yl)-amide

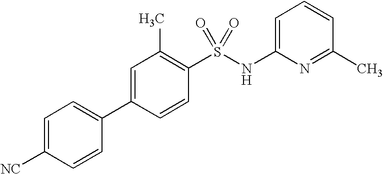

To a mixture of 4-bromo-2-methyl-N-(6-methyl-pyridin-2-yl)-benzenesulfonamide (200 mg, 0.6 mmol) 4-cyanophenyl boronic acid (102 mg, 0.7 mmol) and cesium carbonate (585 mg, 1.8 mmol) in 1,4-dioxane (6 mL) was added [2-[(D-_{κ}N)METHYL]PHENYL-_{κ}C](TRICYCLOHEXYLPHOSPHINE)(TRIFLUOROACETATO-_{κ}O—(SP-4-3)-PALLADIUM, (Bedford, R. B.; Cazin, C. S. J.; Coles, S. J.; Gelbrich, T.; Horton, P. N.; Hursthouse, M. B.; Light, M. E. Organometallics 2003, 22, 987), (2 mg, 0.5 mol %). Mixture heated at reflux for 4 hours. After such time reaction mixture was allowed to cool to ambient temperature, filtered through a pad of Celite® and concentrated in vacuo. Residue was purified by flash column chromatography (SiO₂ 2 g, dichloromenthane, methanol 0% & 1%) to return desired product as a white solid (19 mg, 0.05 mmol, 9% yield).

### Method AA

### Example 282

### 4′-Cyano-3′-methyl-biphenyl-4-sulfonic acid (6-amino-pyridin-2-yl)-amide

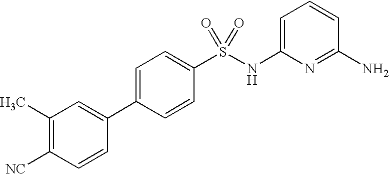

### Preparation of 2-Methyl-4-(4,4,5,5-tetramethyl-[1,3,2]dioxaborolan-2-yl)-benzonitrile

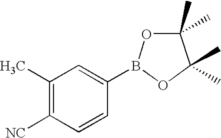

Made following the procedure described for the preparation of N-(6-methyl-pyridin-2-yl)-4-(4,4,5,5-tetramethyl-[1,3,2]dioxaborolan-2-yl)-benzenesulfonamide but substituting 4-bromo-2-methyl-benzonitrile and making non-critical variations. ¹H NMR (400 MHz, CDCl₃), δ ppm 7.63 (s, 1 H) 7.56 (d, J=7.6 Hz, 1 H) 7.45 (d, J=7.6 Hz, 1 H) 2.42 (s, 3 H), 1.24 (s, 12 H).

### 4′-Cyano-3′-methyl-biphenyl-4-sulfonic acid (6-amino-pyridin-2-yl)-amide

Made by following the procedure described for the preparation of 4′chlorobiphenyl-4-sulfonic acid (6-methyl-pyridin-2-yl)-amide but substituting 2-methyl-4-(4,4,5,5-tetramethyl-[1,3,2]dioxaborolan-2-yl)-benzonitrile and N-(6-amino-pyridin-2-yl)-4-bromo-benzenesulfonamide and making non-critical variations.

### Method AB

### Example 283

### 4′-Cyano-3-fluoro-biphenyl-4-sulfonic acid (6-methyl-pyridin-2-yl)-amide

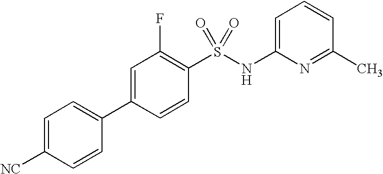

### Preparation of 4-Bromo-2-fluoro-N-(6-methyl-pyridin-2-yl)-benzenesulfonamide

Made following the procedure described for the preparation of 4′-cyano-biphenyl-4-sulfonic acid (6-methyl-pyridin-2-yl)-amide but substituting 4-bromo-2-fluorobenzenesulfonyl chloride and making non-critical variations. The crude material was carried to the next step.

### 4′-Cyano-3-fluoro-biphenyl-4-sulfonic acid (6-methyl-pyridin-2-yl)-amide

Made following the procedure described for the preparation of 4′chlorobiphenyl-4-sulfonic acid (6-methyl-pyridin-2-yl)-amide but substituting 4-bromo-2-fluoro-N-(6-methyl-pyridin-2-yl)-benzenesulfonamide and 4-cyanophenylboronic acid and making non-critical variations.

### Method AC

### Example 284

### 4′-Cyano-2-fluoro-biphenyl-4-sulfonic acid (6-methyl-pyridin-2-yl)-amide

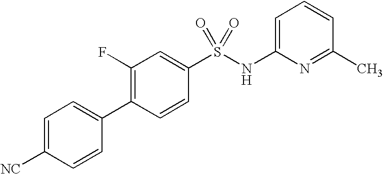

### Preparation of 4-Bromo-3-fluoro-N-(6-methyl-pyridin-2-yl)-benzenesulfonamide

Made by following the procedure described for the preparation of 4′-cyano-biphenyl-4-sulfonic acid (6-methyl-pyridin-2-yl)-amide but substituting 4-bromo-3-(trifluoromethyl)benzenesulfonyl chloride and making non-critical variations. The crude material was carried to the next step.

### 4′-Cyano-2-fluoro-biphenyl-4-sulfonic acid (6-methyl-pyridin-2-yl)-amide

Made by following the procedure described for the preparation of 4′chlorobiphenyl-4-sulfonic acid (6-methyl-pyridin-2-yl)-amide but substituting 4-bromo-3-fluoro-N-(6-methyl-pyridin-2-yl)-benzenesulfonamide and 4-cyanophenylboronic acid and making non-critical variations.

### Method AD

### Example 285

### 4′-Cyano-2-trifluoromethyl-biphenyl-4-sulfonic acid (6-methyl-pyridin-2-yl)-amide

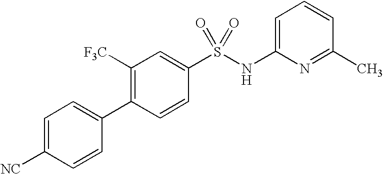

### Preparation of 4-Bromo-N-(6-methyl-pyridin-2-yl)-3-trifluoromethyl-benzenesulfonamide

Made by following the procedure described for the preparation of 4′-cyano-biphenyl-4-sulfonic acid (6-methyl-pyridin-2-yl)-amide but substituting 4-bromo-3-(trifluoromethyl)benzenesulfonyl chloride and making non-critical variations. The crude material was carried to the next step.

### 4′-Cyano-2-trifluoromethyl-biphenyl-4-sulfonic acid (6-methyl-pyridin-2-yl)-amide

Made by following the procedure described for the preparation of 4′chlorobiphenyl-4-sulfonic acid (6-methyl-pyridin-2-yl)-amide but substituting 4-bromo-N-(6-methyl-pyridin-2-yl)-3-trifluoromethyl-benzenesulfonamide and 4-cyanophenylboronic acid and making non-critical variations.

### Method AE

### Example 286

### 4′-Cyano-3-hydroxy-biphenyl-4-sulfonic acid (6-methyl-pyridin-2-yl)-amide

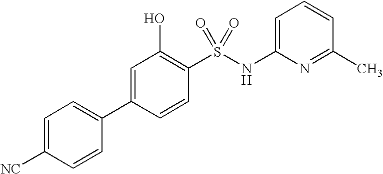

To a solution of 4′-cyano-3-methoxy-biphenyl-4-sulfonic acid (6-methyl-pyridin-2-yl)-amide (28 mg, 0.073 mmol) in CH₂Cl₂ (2 mL) was added BBr₃ (0.2 mL, 1.0 M in CH₂Cl₂) at 0° C. The mixture was warmed to 23° C. and stirred for 1 h. The mixture was then quenched with saturated NaHCO₃ and extracted with EtOAc. The organic layer was dried over sodium sulfate and concentrated to give a residue, which was purified by flash column chromatography to furnish the title compound as a white solid (17 mg, 65% yield).

### Method AF

### Example 287

### 4-Pyridin-2-yl-N-quinolin-2-yl-benzenesulfonamide

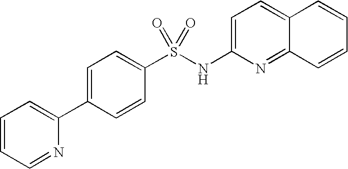

### Preparation of 4-bromo-N-quinolin-2-ylbenzenesulfonamide

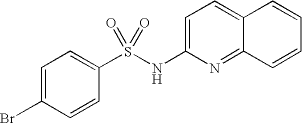

Made by following the procedure described for the preparation of 4′-cyano-biphenyl-4-sulfonic acid (6-methyl-pyridin-2-yl)-amide but substituting 6-bromophenylsulfonyl chloride and 2-aminoquinoline and making non-critical variations. 1H NMR (400 MHz, DMSO-d6), δ ppm 7.37 (t, J=7.58 Hz, 1 H) 7.44-7.51 (m, 1 H) 7.56 (d, J=8.34 Hz, 1 H) 7.64-7.70 (m, 1 H) 7.70-7.74 (m, 2 H) 7.81 (d, J=8.59 Hz, 3 H) 8.23 (d, J=9.60 Hz, 1 H); APCI MS: m/z 365.0 (M+2).

### 4-Pyridin-2-yl-N-quinolin-2-yl-benzenesulfonamide

To a solution of 4-bromo-N-quinolin-2-ylbenzenesulfonamide (50 mg) in 1,4 dioxane (2.0 ml) was added 2-bromopyridine (22 mg), tetrakis(triphenylphosphine)palladium (16 mg), hexamethylditin (50 mg). After the resulting mixture was heated in microwave at 130° C. for 30 mins, it was filtered and concentrated under reduced pressure. To the resulting residue was added 1,4 dioxane (2.0 mL), 2-bromopyridine (30 mg), tetrakis(triphenylphosphine)palladium (20 mg), hexamethylditin (50 mg). After the reaction mixture was heated in microwave at 130° C. for 90 min, it was filtered and concentrated under reduced pressure. The residue was purified using reversed phase Kromasil® C18, 0.05% TFA in water and acetonitrile to provide the titled product (5.4 mg).

### Method AG

### Example 290

### 6-(4-Cyano-phenyl)-pyridine-3-sulfonic acid quinolin-2-ylamide

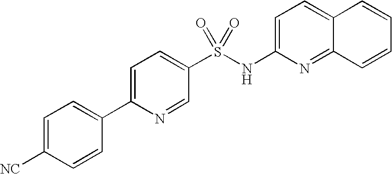

### Preparation of 6-chloro-N-quinolin-2-ylpyridine-3-sulfonamide

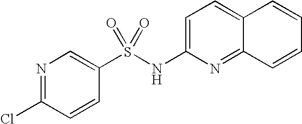

Made by following the procedure describe for the preparation of 4′-cyano-biphenyl-4-sulfonic acid (6-methyl-pyridin-2-yl)-amide but substituting 2-aminoquinoline and 2-chloro-pyridin-5-sulfonyl chloride (Naegeli, C.; Kundig, W.; Brandenburger, H. Helv. Chem. Acta. 1939, 21, 1746) and making non-critical variations. ¹H NMR (400 MHz, DMSO-D6) □ ppm 7.42 (t, J=7.45 Hz, 1 H) 7.55-7.63 (m, 2 H) 7.67-7.74 (m, 2 H) 7.87 (d, J=7.83 Hz, 1 H) 8.28 (d, J=6.82 Hz, 1 H) 8.31 (d, J=9.60 Hz, 1 H) 8.88 (s, 1 H).

### 6-(4-Cyano-phenyl)-pyridine-3-sulfonic acid quinolin-2-ylamide

To a flask containing 6-chloro-N-quinolin-2-ylpyridine-3-sulfonamide (148 mg, 0.46 mmol) and 4-cyanophenylboronic acid (136 mg, 0.92 mmol) were added DME (1.5 mL), N,N-dimethylacetamide (2.0 mL), H₂O (0.5 mL), Cs₂CO₃ (451 mg, 1.39 mmol). The reaction mixture was degassed by alternating between vacuum and nitrogen. After [1,1-bis(diphenylphosphino)-ferrocene]dichloropalladium (II)-dicholoromethane complex (16 mg) was added, the reaction mixture was degassed again. After the resulting mixture was heated at 80° C. for 19 hours, it was diluted with EtOAc (30 mL), sat NaHCO₃ (5 mL). After the resulting mixture was stirred at R.T. for 5 min, it was filtered and diluted with sat NaHCO₃ (5 mL). The layers were separated. The aqueous layer was extracted with EtOAc (2×15 mL). The combined organic extracts were dried with K₂CO₃, filtered, and concentrated to give a solid. After triturating the resulting solid with CH₂Cl₂, the desired product was obtained (59.7 mg). The mother liquor was purified using high performance flash chromatography (0→30% dichloromethane in acetone) to give an additional batch of desired product (33.3 mg).

### Method AH

### Example 293

### 6-(4-Cyano-phenyl)-pyridine-3-sulfonic acid (6-cyclopropyl-pyridin-2-yl)-amide

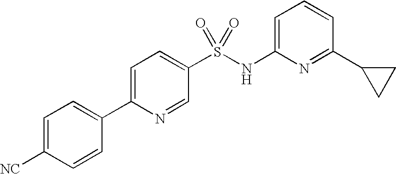

### Preparation of 6-Chloro-pyridine-3-sulfonic acid (6-cyclopropyl-pyridin-2-yl)-amide

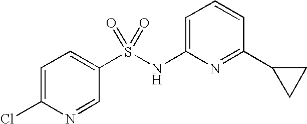

Made by following the procedure described for the preparation of 4′-cyano-biphenyl-4-sulfonic acid (6-methyl-pyridin-2-yl)-amide but substituting 6-cyclopropyl-pyridin-2-ylamine and 6-chloro-3-pyridylsulfonyl chloride (Naegeli, C.; Kundig, W.; Brandenburger, H. Helv. Chem. Acta. 1939, 21, 1746) and making non-critical variations. ¹H NMR (400 MHz, CDCl₃), δ: 8.91 (d, J=2.5 Hz, 1 H), 8.18 (dd, J=8.4, 2.5 Hz, 1 H), 7.53 (t, J=7.5 Hz, 1 H), 7.43 (d, J=8.3 Hz, 1 H), 6.89 (d, J=8.6 Hz, 1 H), 6.55 (d, J=7.3 Hz, 1 H), 6.27 (d, J=8.1 Hz 1 H), 1.98-1.92 (m, 1 H), 1.14-1.09 (m, 2 H) 0.93-0.89 (m, 2 H); LCMS (ESI): 310.1.

### 6-(4-Cyano-phenyl)-pyridine-3-sulfonic acid (6-cyclopropyl-pyridin-2-yl)-amide

Made by following the procedure described for the preparation of 4′chlorobiphenyl-4-sulfonic acid (6-methyl-pyridin-2-yl)-amide but substituting 6-chloro-pyridine-3-sulfonic acid (6-cyclopropyl-pyridin-2-yl)-amide and 4-cyanophenyl boronic acid and making non-critical variations.

### Method AI

### Example 295

### 5-Cyano-3-methyl-benzo[b]thiophene-2-sulfonic acid (6-methyl-pyridin-2-yl)-amide

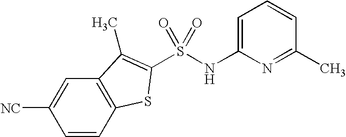

### Preparation of 5-Bromo-3-methyl-benzo[b]thiophene-2-sulfonic acid (6-methyl-pyridin-2-yl)-amide

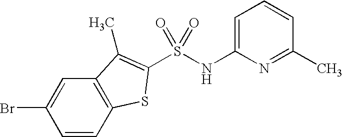

Made by following the procedure described for the preparation of 4′-cyano-biphenyl-4-sulfonic acid (6-methyl-pyridin-2-yl)-amide but substituting 5-bromo-3-methyl-benzo[b]thiophene-2-sulfonyl chloride and making non-critical variations. ¹H NMR (400 MHz, CDCl₃), δ: 7.88 (d, J=1.8 Hz, 1 H), 7.62 (d, J=8.6 Hz, 1 H), 7.47-7.58 (m, 2 H), 7.11 (d, J=9.1 Hz, 1 H), 6.54 (d, J=7.3 Hz, 1 H), 2.68 (s, 3 H), 2.51 (s, 3 H); MS (ESI) for C₁₅H₁₄BrN₂O₂S₂ m/z: 398.0.

### 5-Cyano-3-methyl-benzo[b]thiophene-2-sulfonic acid (6-methyl-pyridin-2-yl)-amide

Copper (I) cyanide (43 mg, 0.476 mmol, 1.5 equiv) was added to a solution of 5-bromo-3-methyl-benzo[b]thiophene-2-sulfonic acid (6-methyl-pyridin-2-yl)-amide (126 mg, 0.317 mmol, 1 equiv) in dimethylformamide (2.5 mL) at 24° C. The solution was heated to 250° C. by microwave for 10 min. Deionized water (5 mL), hexanes (2.5 mL), and diethyl ether (2.5 mL) were added, and the resulting tan solid was collected by filtration. Purification of the solid by preparative reverse phase HPLC (Kromasil® C18, 10 μm, 250×50.8 mm, mobile phase: water/acetonitrile/0.05% trifluoroacetic acid) provided the titled compound (30 mg, 27.5%).

### Method AJ

### Example 296

### Pyrrolidine-2-carboxylic acid [6-(3-chloro-2-methyl-benzenesulfonylamino)-pyridin-2-yl]-amide

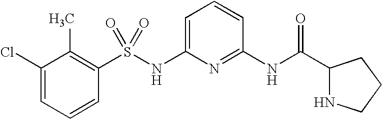

A mixture of (6-amino-pyridin-2-yl)-3-chloro-2-methyl-benzenesulfonamide (140 mg, 0.47 mmol), pyrrolidine-1,2-dicarboxylic acid 1-tert-butyl ester (106 mg, 0.50 mmol), HATU (215 mg, 0.57 mmol) and Et₃N (0.2 mL) in DMF (3 mL) was stirred at 23° C. for 12 h. The mixture was partitioned between EtOAc and water. The organic layer was dried and concentrated to give the crude amide as an oil, which was used directly in the next reaction. The amide was dissolved in CH₂Cl₂ (2 mL), and HCl (4 ml; 4 N in dioxane) was added. The mixture was stirred at 23° C. for 12 h. The mixture was concentrated and the residue was purified by reverse-phase HPLC to give the title compound as a white solid (99 mg, 53%).

### Method AK

### Example 297

### 3-Pyridin-4-yl-pyrrolidine-1-sulfonic acid (6-methyl-pyridin-2-yl)-amide

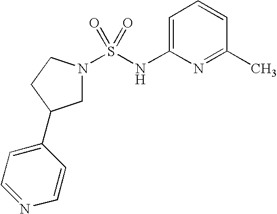

### Preparation of N-(6-methylpyridin-2-yl)-2-oxo-1,3-oxazolidine-3-sulfonamide

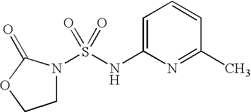

Chlorosulfonyl isocyanate (0.27 mL, 4.1 mmol) was dissolved in 40 mL of CH₂Cl₂ and cooled to 0° C. Chloroethanol (0.27 mL, 4.1 mmol) was added slowly and the reaction mixture was stirred at 0° C. for 1.5 h. A solution of 6-methyl-2-aminopyridine (444 mg, 4.1 mmol) and Et3N (1.3 ml, 12.4 mmol) in 50 mL of CH₂Cl₂ was slowly added so that the reaction temperature did not exceed 5° C. The reaction solution was slowly warmed to room temperature and stirred overnight. After acidic workup, the crude product was purified by triturating with CH₂Cl₂ and hexane. ¹H NMR (400 MHz, CDCl₃) δ: 12.34 (s, 1 H) 7.62 (dd, J=8.8, 7.3 Hz, 1 H) 6.77 (d, J=8.8 Hz, 1 H) 6.57 (d, J=7.1 Hz, 1 H) 4.39 (t, J=8.0 Hz, 2 H) 4.15 (t, J=7.8 Hz, 2 H) 2.50 (s, 3 H).

### 3-Pyridin-4-yl-pyrrolidine-1-sulfonic acid (6-methyl-pyridin-2-yl)-amide

A solution of N-(6-methylpyridin-2-yl)-2-oxo-1,3-oxazolidine-3-sulfonamide (0.23 g, 0.894 mmol), 4-pyrrolidin-3-ylpyridine (0.40 g, 2.23 mmol), and diisopropylethylamine (1 mL) in acetonitrile (3 mL) was heated to 130° C. using microwave heating for 0.5 hour. The reaction mixture was cooled to 25° C., and diluted with ethyl acetate (50 mL). The resulting mixture was washed with saturated aqueous ammonium chloride (2×30 mL) and saturated aqueous sodium bicarbonate (2×30 mL). The organic layer was concentrated to give a clear oil. The residue was purified using radial chromatography (2 mm silica plate; 1:1:0.1 dichloromethane/ethyl acetate/methanol). The product was triturated with additional diethyl ether and dried in vacuo to afford the title compound (0.19 g, 65.4%). Sulfamide formation may also occur without microwave by heating the reaction overnight to 82° C. in acetonitrile or 110° C. in dimethylformamide.

### Method AL

### Example 317

### 4-(4-Cyano-phenyl)-piperidine-1-sulfonic acid (6-amino-pyridin-2-yl)-amide

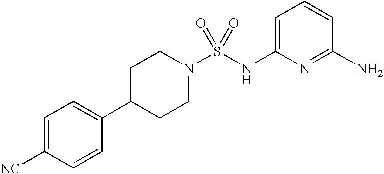

### Preparation of tert-Butyl (6-{[(2-oxo-1,3-oxazolidin-3-yl)sulfonyl]amino}pyridin-2-yl)carbamate

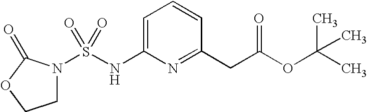

Made by following the procedure described for the preparation of N-(6-methylpyridin-2-yl)-2-oxo-1,3-oxazolidine-3-sulfonamide but substituting tert-butyl (6-aminopyridin-2-yl)carbamate (Berl, et al Chem Eur J 2001, 7, 2798) and making non-critical variations. ¹H NMR (400 MHz, CD₂Cl₂), δ: 1.50 (s, 9 H) 4.05-4.11 (m, 2 H) 4.24-4.30 (m, 2 H) 6.64 (d, J=7.83 Hz, 1 H) 7.32 (d, J=8.08 Hz, 1 H) 7.50 (t, J=8.08 Hz, 1 H).

### 4-(4-Cyano-phenyl)-piperidine-1-sulfonic acid (6-amino-pyridin-2-yl)-amide

A solution of tert-butyl (6-{[(2-oxo-1,3-oxazolidin-3-yl)sulfonyl]amino}pyridin-2-yl)carbamate (150 mg, 0.420 mmol), diisopropylethylamine (219 μL, 1.26 mmol), and 4-(4-cyanophenyl)piperidine (82 mg, 0.44 mmol) was subjected to microwaves at 110° C. for 30 min. The reaction mixture was concentrated and the crude product was purified by flash chromatography eluting with hexanes/ethyl acetate (0-25%). To a cooled (0-5° C.) solution of the afforded material in CH₂Cl₂ (1 mL) was added TFA (1 mL). After 2 hours, the reaction mixture was concentrated and the residue was partitioned between EtOAc (50 mL) and saturated NaHCO₃ (10 mL). The organic layer was separated and washed with brine (10 mL), dried (MgSO4), filtered, and concentrated in vacuo. The crude product was purified by flash chromatography eluting with CH₂Cl₂/MeOH (0-5%) to afford the title compound (30 mg, 20%).

The structure, name, physical and biological data, and Methods are further described in tabular form below in Table 1.

TABLE 1

| | | % | | | | |
|---|---|---|---|---|---|---|
| | | inh | | | | |
| | Ki | @ | | | | |
| | app | 0.1 | | | | MS |
| Eg. | (nM) | uM | Structure | Mth. | ¹H NMR | (m/z) |
| 1 | 42 | 72.3 | 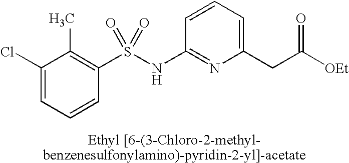 | A | (400 MHz, CDCl₃) δ: 8.02 (dd, J=7.96, 1.14 Hz, 1 H), 7.52 (dd, J=8.46, 7.45 Hz, 2 H), 7.22 (t, J=7.96 Hz, 1 H), 7.01 (d, J=8.34 Hz, 1 H), 6.80 (d, J=7.33 Hz, 1 H), 4.17 (q, J=7.07 Hz, 2 H), 3.68 (s, 2H), 2.73 (s, 3 H), 1.25 (t, J=7.07 Hz, 3H) | 369.0677 |
| 2 | 16 | 85.4 | 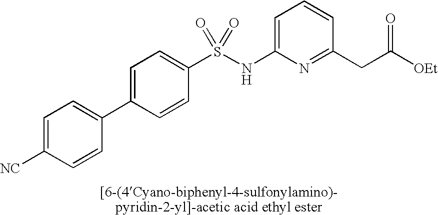 | A | (400 MHz, CDCl₃) δ: 8.02 (d, J=8.6 Hz, 2 H), 7.74 (m, 2 H), 7.66 (d, J=7.8 Hz, 4 H), 7.58 (m, 1 H), 7.20 (d, J=8.3 Hz, 1 H), 6.88 (d, J=27.3 Hz, 1 H), 4.14 (q, J=7.1 Hz, 2 H), 3.67 (s, 2 H), 1.21 (t, J =7.1 Hz, 3 H) | 422.1 |
| 3 | NA | 19.6 | 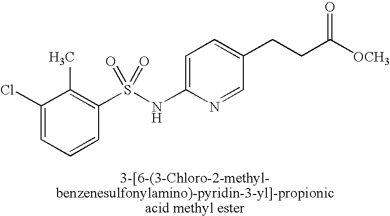 | A | (400 MHz, CDCl₃) δ: 14.18 (s, 1 H) 8.14 (d, J=1.8 Hz, 1 H) 8.08 (m, 1 H) 7.56 (dd, J=9.3, 2.3 Hz, 1 H) 7.48-7.53 (m, 1 H) 7.20-7.30 (m, 2 H) 3.64 (s, 3 H) 2.81 (t, J=7.3 Hz, 2 H) 2.66 (s, 3 H) 2.55 (t, J=7.3 Hz, 2 H) | NA |
| 4 | NA | 23.9 | 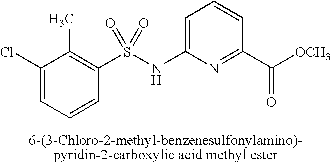 | A | (500 MHz, CDCl₃) δ: 8.04 (d, J=8.1 Hz, 1 H), 7.60-7.75 (m, 2 H), 7.55 (d, J =7.8 Hz, 1 H), 7.30 (d, J =8.4 Hz, 1 H), 7.20-7.27 (m, 1 H), 3.97 (s, 3 H), 2.74 (s, 3 H) | 341.0359 |
| 5 | NA | 0.3 | 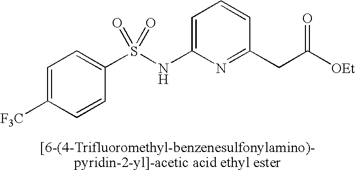 | A | (400 MHz, CDCl₃) δ: 9.03 (br s, 1 H), 8.06 (d, J=8.1 Hz, 2 H), 7.72 (d, J=8.3 Hz, 2 H), 7.58 (m, 1 H), 7.12 (d, J=8.3 Hz, 1 H), 6.83 (d, J=7.3 Hz, 1 H), 4.16 (q, J=7.2 Hz, 2 H), 3.66 (s, 2 H), 1.23 (t, J =7.1 Hz, 3 H) | 389.0789 |
| 6 | 2.8 | 100 | 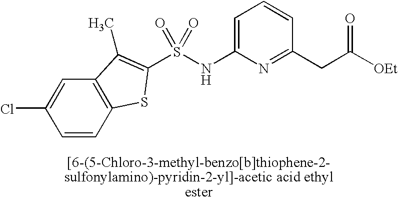 | A | (400 MHz, CDCl₃) δ: 9.31 (br s, 1 H), 7.65-7.75 (m, 2 H), 7.58 (dd, J=8.5, 7.5 Hz, 1 H), 7.40 (dd, J=8.7, 1.9 Hz, 1 H), 7.24 (m, 1 H), 6.81 (d, J=7.3 Hz, 1 H), 4.14 (q, J=7.1 Hz, 2 H), 3.68 (a, 2 H), 2.63 (s, 3 H), 1.22 (t, J = 7.1 Hz, 3 H) | 425.0 |
| 7 | NA | 7.7 | 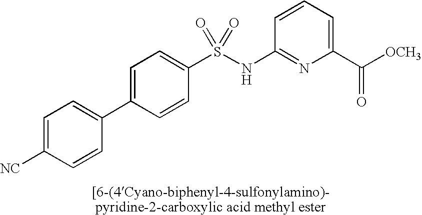 | A | NA | 393.9 |
| 8 | NA | 5.52 | 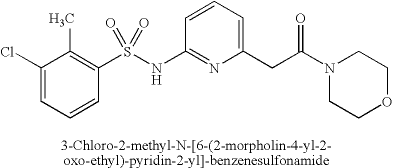 | B | (400 MHz, CDCl₃) δ: 8.01 (dd, J=8.08, 1.01 Hz, 1 H), 7.53 (t, J=8.08 Hz, 2 H), 7.23 (m, 1 H), 7.03 (d, J=8.34 Hz, 1 H), 6.84 (d, J=7.33 Hz, 1 H), 3.77 (s, 2 H), 3.63 (m, 4 H), 3.56 (m, 2 H) | 410.0936 |
| 9 | 169 | 54.8 | 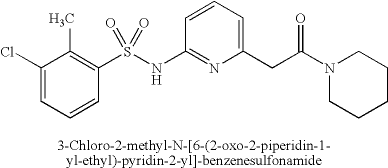 | B | (400 MHz, CDCl₃) δ: 9.57 (br s, 1 H), 8.02 (m, 1 H), 7.37-7.59 (m, 2 H), 7.21 (t, J=8.1 Hz, 1 H), 7.02 (d, J=8.6 Hz, 1 H), 6.75 (d, J=7.3 Hz, 1 H), 3.76 (s, 2 H), 3.54 (m, 2 H), 3.39 (m, 2 H), 2.73 (s, 3 H) 1.33-1.67 (m, 6 H) | 408.1169 |
| 10 | NA | 38.7 | 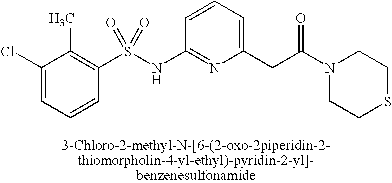 | B | (400 MHz, CDCl₃) δ: 8.01 (dd, J=8.0, 1.1 Hz, 1 H), 7.42-7.59 (m, 2 H), 7.22 (t, J=8.0 Hz, 1 H), 7.06 (d, J=8.6 Hz, 1 H), 6.79 (d, J=7.3 Hz, 1 H), 3.77-3.90 (m, 4 H), 3.72 (m, 2 H), 2.70 (s, 3 H), 2.57 (m, 2 H), 2.46 (m, 2 H) | 426.0715 |
| 11 | NA | 8.05 | 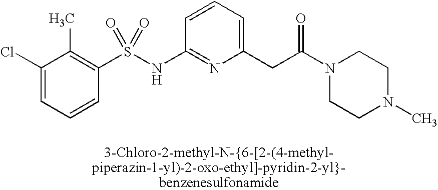 | B | (400 MHz, CDCl₃) δ: 8.02 (dd, J=8.1, 1.0 Hz, 1 H), 7.41-7.60 (m, 2 H), 7.19-7.24 (m, 1 H), 7.01 (d, J =8.3 Hz, 1 H), 6.80 (d, J =7.3 Hz, 1 H), 3.76 (s, 2 H); 3.65 (br s, 2 H), 3.53 (br s, 2 H), 2.73 (s, 3 H), 2.18-2.49 (m, 7 H) | 423.1251 |
| 12 | NA | 21.5 | 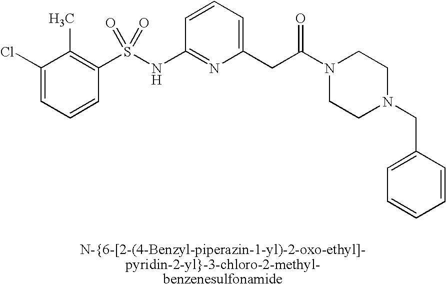 | B | (400 MHz, CDCl₃) δ: 8.00 (m, 1 H), 7.49 (t, J=8.0 Hz, 2 H), 7.22-7.34 (m, 5 H), 7.19 (t, J=7.8 Hz, 1 H), 7.04 (d, J=8.3 Hz, 1 H), 6.74 (d, J=7.1 Hz, 1 H), 3.80 (s, 2 H), 3.60 (m, 2 H), 3.48 (s, 2 H), 3.44 (m, 2 H), 3.48 (s, 2 H), (m, 2 H), 2.32 (m, 2 H) | 499.1554 |
| 13 | NA | 9.1 | 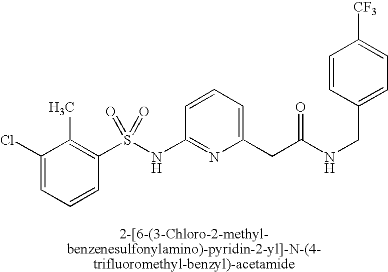 | B | NA | 498.0870 |
| 14 | NA | 25.9 | 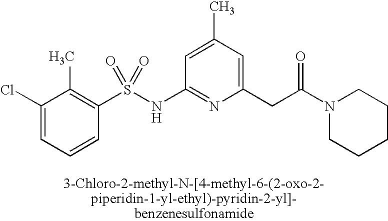 | B | (400 MHz, CDCl₃) δ: 8.03 (dd, J=8.0, 1.1 Hz, 1 H), 7.50 (dd, J=8.0, 1.1 Hz, 1 H), 7.15-7.23 (m, 1 H), 6.84 (s, 1 H), 6.52 (s, 1 H), 3.70 (s, 2 H), 3.55 (m, 2 H), 3.41 (m, 2 H), 2.75 (s, 3 H), 2.23 (s, 3 H), 1.62 (m, 2 H), 1.44-1.58 (m, 4 H) | 422.1295 |
| 15 | NA | 23.6 | 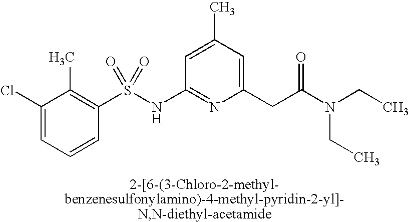 | B | (400 MHz, CDCl₃) δ: 10.29 (br s, 1 H), 8.04 (m, 1 H), 7.48 (dd, J=8.1, 1.0 Hz, 1 H), 7.19 (t, J=8.0 Hz, 1 H), 6.84 (s, 1 H), 6.46 (s, 1 H), 3.66 (s, 2 H), 3.28-3.44 (m, 4 H), 2.75 (s, 3 H), 2.22 (s, 3 H), 1.17 (t, J=7.2 Hz, 3 H), 1.12 (t, J=7.2 Hz, 3 H) | 410.1291 |
| 16 | NA | 10.9 | 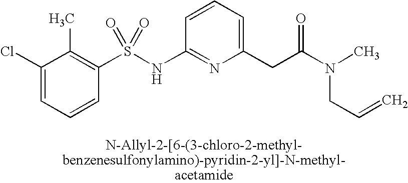 | B | (1:1 rotamer ratio, 400 MHz, CDCl₃) δ: 7.96-8.06 (m, 1 H), 7.42-7.57 (m, 2 H), 7.16 -7.23 (m, 1 H), 6.96-7.10 (m, 1 H), 6.69-6.78 (m, 1 H), 5.63-5.79 (m, 1 H), 5.03-5.25 (m, 2 H), 3.90-4.02 (m, 2 H), 3.81 (s, 2 H), 3.74 (s, 2 H), 2.96 (s, 3 H), 2.93 (s, 3 H), 2.73 (s, 3 H), 2.72 (s, 3 H) | 394.2 |
| 17 | NA | 11.4 | 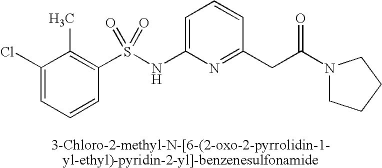 | B | (400 MHz, CDCl₃) δ: 8.02 (m, 1 H), 7.42-7.56 (m, 2 H), 7.19 (t, J=8.0 Hz, 1 H), 7.04 (d, J=8.6 Hz, 1 H), 6.69 (d, J=7.3 Hz, 1 H), 3.72 (s, 2 H), 3.46 (t, J=6.7 Hz, 4 H), 2.73 (s, 3 H), 1.78-2.02 (m, 4 H) | 394.0988 |
| 18 | NA | 34.6 | 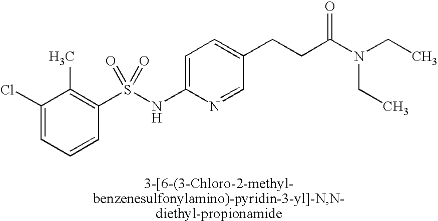 | B | (400 MHz, CDCl₃) δ: 13.56 (s, 1 H) 8.01-8.13 (m, 2 H) 7.62 (dd, J=9.1, 2.3 Hz, 1 H) 7.48-7.54 (m, 1 H) 7.15-7.30 (m, 2 H) 3.32 (q, J=7.1 Hz, 2 H) 3.21 (q, J=7.1 Hz, 2 H) 2.85 (t, J=7.2 Hz, 2 H) 2.68 (s, 3 H) 2.52 (t, J=7.2 Hz, 2 H) 1.02-1.15 (m, 6 H) | NA |
| 19 | 4.8 | 96.9 | 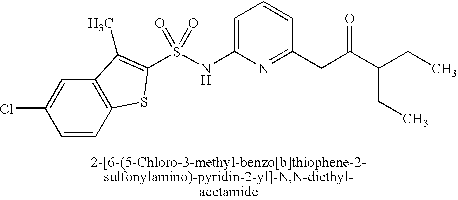 | C | (400 MHz, CDCl₃) δ: 10.72 (br s, 1 H), 7.61-7.71 (m, 2 H), 7.61-7.71 (m, 2 H), 7.55 (dd, J=8.7, 7.5 Hz, 1 H), 7.36 (dd, J=8.6, 2.0 Hz, 1 H), 7.27 (d, J=8.8 Hz, 1 H), 6.72 (d, J=H), 7.55 (dd, J=8.7, 7.5 Hz, 1 2H), 7.36 (dd, J=8.6, 2.0 Hz, 1 H), 7.27 (d, J=8.8 Hz, 1 H), 6.72 (d, J=7.1 Hz, 1 H), 3.77 (s, 2 H), | |
| # 3.28-3.40 (m, 4 H), 2.62 (s, 3 H), 1.04-1.16 (m, 6 H) | 452.1 | | | | | |
| 20 | 220 | NA | 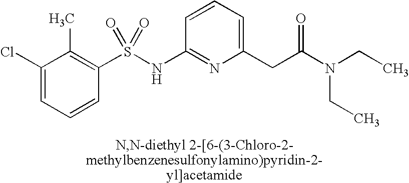 | C | (400 MHz, CDCl₃) δ: 9.80 (br s, 1 H), 8.04 (m, 1 H), 7.41-7.58 (m, 2 H), 7.20 (t, J=7.8 Hz, 1 H), 7.01 (d, J=8.6 Hz, 1 H), 6.72 (d, J=7.3 Hz, 1 H), 3.69 (s, 2 H), 3.31-3.41 (m, 4H), 2.75 (s, 3 H), 1.07-1.17 (m, 6 H) | 396.1146 |
| 21 | 480 | NA | 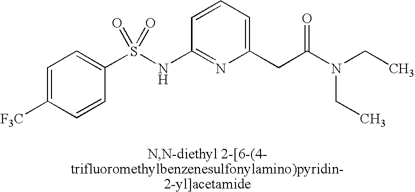 | C | (400 MHz, CDCl₃), δ: 10.28 (br s, 1 H), 8.09 (d, J=8.3 Hz, 2 H), 7.70 (d, J=8.1 Hz, 2 H), 7.54 (dd, J=8.5, 7.5 Hz, 1 H), 7.10 (d, J =8.6 Hz, 1 H), 6.70 (d, J =7.3 Hz, 1 H), 3.70 (s, 2 H), 3.39 (q, J=7.1 Hz, 2 H), 3.33 (q, J=7.2 Hz, 2 H), 1.04-1.19 (m, 6 H) | 416.3 |
| 22 | 170 | 44.7 | 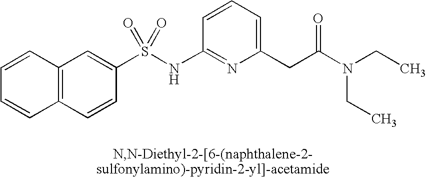 | C | (400 MHz, CDCl₃), δ: 8.60 (br s, 1 H), 8.52 (s, 1 H), 7.76-8.00 (m, 4 H), 7.43-7.69 (m, 3 H), 7.19 (d, J =8.3 Hz, 1 H), 6.84 (d, J =7.6 Hz, 1 H), 3.66 (s, 2 H), 3.32 (q, J=7.1 Hz, 2 H), 3.23 (q, J=7.2 Hz, 2 H), 1.05 (t, J=7.1 Hz, 3 H), 1.00 (t, J=7.2 Hz, 3 H) | 398.2 |
| 23 | NA | 4.9 | 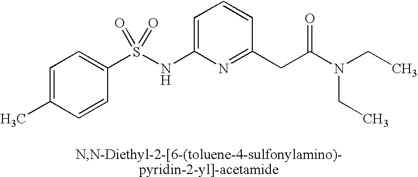 | C | (400 MHz, CDCl₃) δ: 7.78 (d, J=7.8 Hz, 2 H), 7.53 (t, J=7.8 Hz, 1 H), 7.24 (m, 2 H), 7.15 (d, J=8.3 Hz, 1 H), 6.90 (d, J=7.3 Hz, 1 H), 3.70 (s, 2 H), 3.21-3.44 (m, 4 H), 2.37 (s, 3 H), 1.00-1.14 (m, 6 H) | 362.1538 |
| 24 | NA | 3.7 | 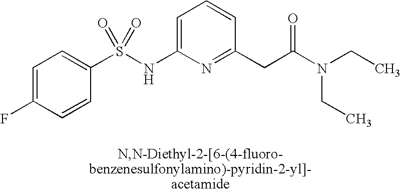 | C | (400 MHz, CDCl₃) δ: 8.88 (br s, 1 H), 7.94 (dd, J =8.7, 4.9 Hz, 2 H), 7.54 (m, 1 H), 7.02-7.19 (m, 3 H), 6.83 (d, J=7.3 Hz, 1 H), 3.69 (s, 2 H), 3.37 (q, J =7.1 Hz, 2 H), 3.31 (q, J =7.2 Hz, 2 H), 1.10 (m, 6 H) | 366.1272 |
| 25 | NA | 42.8 | 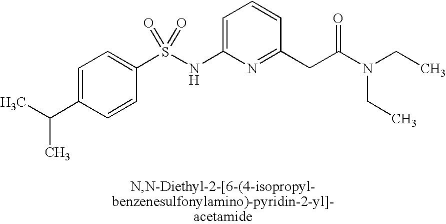 | C | (400 MHz, CDCl₃) δ: 7.87 (d, J=81 Hz, 2 H), 7.75 (m, 1 H), 7.44 (m, 1 H), 7.33 (d, J=8.1 Hz, 2 H), 7.09 (m, 1 H), 3.82 (s, 2 H), 325-3.46 (m, 4 H), 2.94 (m, 1 H), 1.23 (d, J =7.1 Hz, 6 H), 1.03-1.18 (m, 6 H) | 390.1837 |
| 26 | NA | 25.2 | 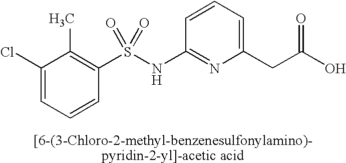 | D | (400 MHz, CDCl₃) δ: 8.05 (m, 1 H), 7.84 (m, 1 H), 7.57 (m, 1 H), 7.24 (m, 1 H), 7.10 (d, J=8.3 Hz, 1 H), 6.87 (d, J=7.3 Hz, 1 H), 3.76 (s, 2 H), 2.73 (s, 3 H) | 341.0371 |
| 27 | NA | 1.9 | 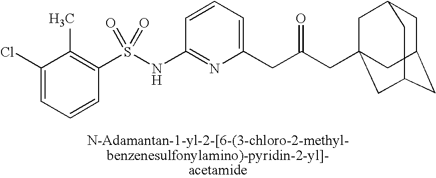 | E | (1:1 rotamer ratio, 400 MHz, CDCl₃) δ: 8.06 (m, 1 H), 7.54 (m, 2 H), 7.24 (m, 1 H), 7.09 (d, J=8.6 Hz, 1 H), 6.84 (d, J=7.3 Hz, 1 H), (s, 1 H), 3.53 (s, 2 H), 2.75 (s, 3 H), 2.03 (s, 3 H), 1.92 (d, J=2.5 Hz, 6 H), 1.64 (m, 6 H) | 474.3 |
| 28 | NA | 45.6 | 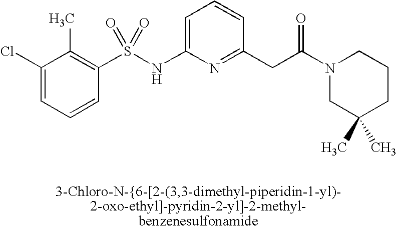 | E | NA | 436 |
| 29 | NA | 10.9 | 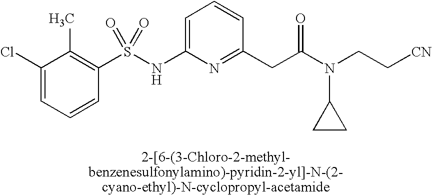 | E | NA | 433 |
| 30 | NA | 15.9 | 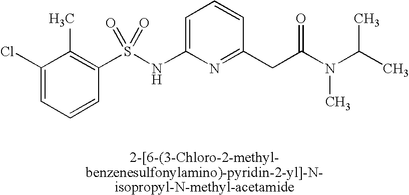 | E | NA | 396.1 |
| 31 | NA | 11.3 | 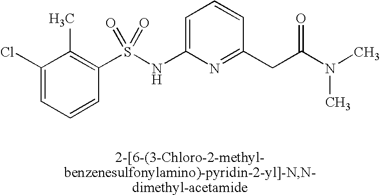 | E | NA | 368 |
| 32 | NA | 20.4 | 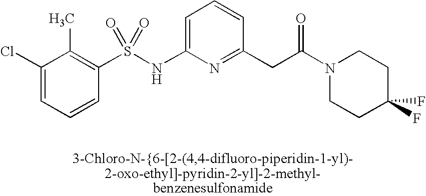 | E | NA | 443.9 |
| 33 | 6.4 | 97 | 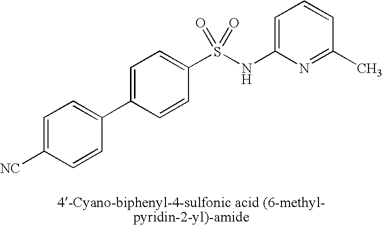 | F | (400 MHz, CDCl₃) δ: 2.42 (s, 3 H), 6.59 (d, J=6.8 Hz, 1 H), 6.97 (m, 1 H), 7.52 (dd, J=8.8, 7.73 Hz, 1 H), 7.67 (m, 4 H), 7.75 (m, 2 H), 8.05 (m, 1 H) | 350.1 |
| 34 | 169 | 48.8 | 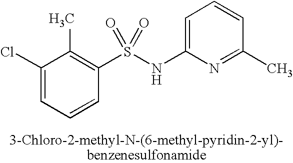 | F | (400 MHz, CDCl₃) δ: 8.02 (d, J=7.1 Hz, 1 H), 7.41-7.55 (m, 2 H), 7.20 (t, J =8.0 Hz, 1 H), 6.93 (d, J =8.8 Hz, 1 H), 6.51 (d, J =7.1 Hz, 1 H), 2.77 (s, 3 H), 2.49 (s, 3 H) | 297.2 |
| 35 | 108 | 98 | 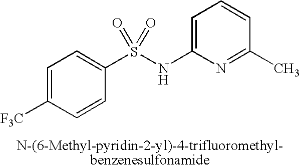 | F | (400 MHz, CDCl₃) δ: 8.06 (d, 2 H, J=8.08 Hz), 7.70 (d, 2 H, J=8.08 Hz), 7.55 (m, 1 H), 7.05 (d, 1 H, J =8.84 Hz), 6.57 (d, 1 H, J =7.07 Hz), 2.48 (s, 3H) | 317.0566 |
| 36 | 48 | 52 | 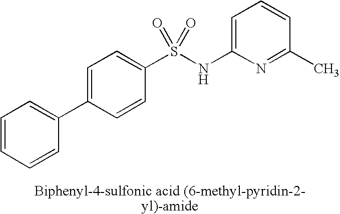 | F | (400 MHz, CDCl₃) δ: 8.00 (m, 2 H), 7.67 (m, 2 H), 7.50-7.59 (m, 3 H), 7.35-7.49 (m, 3 H), 7.09 (d, J =8.6 Hz, 1 H), 6.63 (d, J =7.3 Hz, 1 H), 2.44 (s, 3 H) | 325.1019 |
| 37 | 84 | 45 | 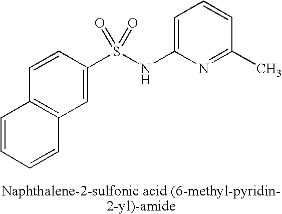 | F | (400 MHz, CDCl₃) δ: 8.51 (s, 1 H), 7.77-8.00 (m, 4 H), 7.58, (m, 2 H), 7.49 (dd, J=8.6, 7.3 Hz, 1 H), 7.13 (d, J=8.6 Hz, 1 H), 6.57 (d, J=7.3 Hz, 1 H), 2.44 (s, 3 H) | 299.0859 |
| 38 | 169 | 49 | 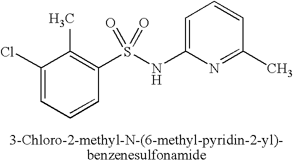 | F | (400 MHz, CDCl₃) δ: 8.02 (d, J=7.1 Hz, 1 H), 7.41-7.55 (m, 2 H), 7.20 (t, J =8.0 Hz, 1 H), 6.93 (d, J =8.8 Hz, 1 H), 6.51 (d, J =7.1 Hz, 1 H), 2.77 (s, 3 H), 2.49 (s, 3 H) | 297.0458 |
| 39 | 9 | 96 | 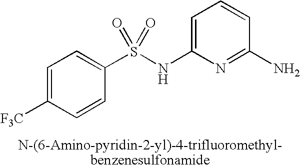 | F | (400 MHz, pyridine-d₅) δppm 5.92 (d, J=8.34 Hz, 1 H) 6.20 (d, J=8.34 Hz, 1 H) 7.27 (t, J=8.21 Hz, 1 H) 7.70 (d, J=8.34 Hz, 2 H), 7.98 (d, J=8.34 Hz) | 318.1 |
| 40 | 4.3 | 98 | 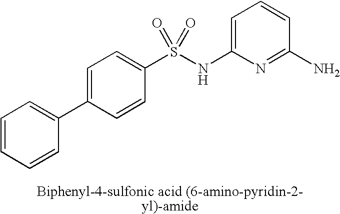 | F | (400 MHz, CDCl₃) δ: 5.93 (d, J=8.1 Hz, 1 H), 6.23 (d, J=8.1 Hz, 1 H), 7.24 (t, J=8.3 Hz, 1 H), 7.28 (m, 1 H), 7.35 (t, J=7.3 Hz, 2 H), 7.54 (d, J =7.1 Hz, 2 H), 7.64 (d, J=8.6 Hz, 2 H), 7.87 (d, J=8.6 Hz, 2 H) | 326.1 |
| 41 | 17 | 94 | 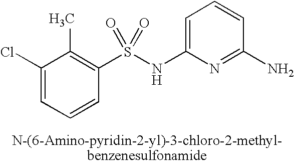 | F | (400 MHz, CDCl₃) δ: 7.90 (m, 1 H), 7.45 (d, J=7.8 Hz, 1 H), 7.24 (t, J=8.2 Hz, 1 H), 7.18 (t, J=8.1 Hz, 1 H), 6.13 (d, J=7.6 Hz, 1 H), 5.87 (d, J=8.1 Hz, 1 H), 2.62 (s, 3 H) | 298.1 |
| 42 | 4.6 | 96 | 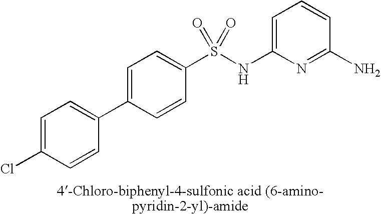 | F | (400 MHz, CDCl₃) δ: 5.93 (d, J=8.1 Hz, 1 H), 6.23 (d, J=8.1 Hz, 1 H), 7.24 (t, J=8.3 Hz, 1 H), 7.28 (m, 1 H), 7.35 (t, J=7.3 Hz, 2 H), 7.54 (d, J=7.07 Hz, 2 H), 7.64 (d, J=8.6 Hz, 2 H), 7.87 (d, J=8.6 Hz, 2 H) | NA |
| 43 | 8.1 | 98 | 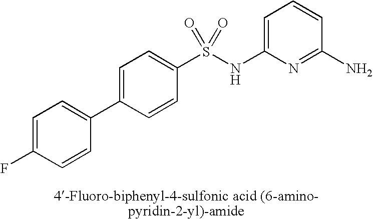 | F | (400 MHz, MeOD) δ: 7.96 (d, J=8.3 Hz, 2 H), 7.70 (d, J=8.3 Hz, 2 H), 7.60-7.68 (m, 2 H), 7.33 (t, J=8.1 Hz, 1 H), 7.18 (t, J=8.7 Hz, 2 H), 6.33 (d, J=8.1 Hz, 1 H), 6.03 (d, J=8.3 Hz, 1H) | NA |
| 44 | NA | 16 | 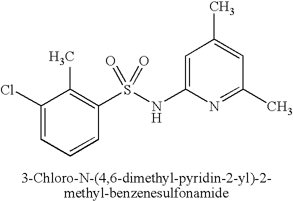 | F | (400 MHz, CDCl₃) δ: 8.01 (m, 1 H), 7.48 (m, 1 H), 7.19 (m, 1 H), 6.74 (s, 1 H), 6.32 (s, 1 H), 2.77 (s, 3 H), 2.43 (s, 3 H), 2.23 (s, 3 H) | 311.0612 |
| 45 | NA | 35 | 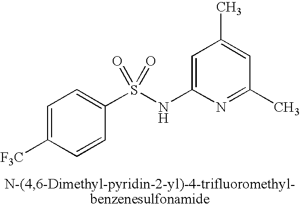 | F | (400 MHz, CDCl₃) δ: 11.59 (br s, 1 H), 8.05 (d, J ==8.3 Hz, 2 H), 7.69 (d, J=8.1 Hz, 2 H), 6.80 (s, 1 H), 6.35 (s, 1 H), 2.43 (s, 3 H), 2.25 (s, 3 H) | 331.0738 |
| 46 | 3.2 | 98 | 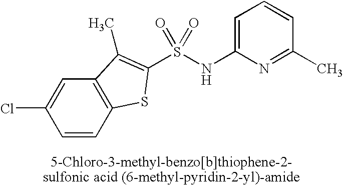 | F | (400 MHz, CDCl₃) δ: 7.65-7.73 (m, 2 H), 7.56 (dd, J =8.8, 7.3 Hz, 1 H), 7.38 (dd, J=8.6, 2.0 Hz, 1 H), 7.14 (d, J=8.8 Hz, 1 H), 6.55 (d, J=7.3 Hz, 1 H), 2.68 (s, 3 H), 2.52 (s, 3 H) | 353.0197 |
| 47 | NA | 21 | 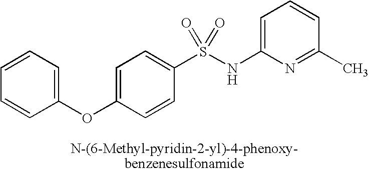 | F | (400 MHz, CDCl₃) δ: 9.65 (br s, 1 H), 7.86 (m, 2 H), 7.49 (dd, J=8.6, 7.3 Hz, 1 H), 7.37 (m, 2 H), 7.18 (t, J=7.5 Hz, 1 H), 6.91-7.06 (m, 5 H), 6.62 (d, J =7.3 Hz, 1 H), 2.41 (s, 3 H) | 341.0946 |
| 48 | 14.5 | 90 | 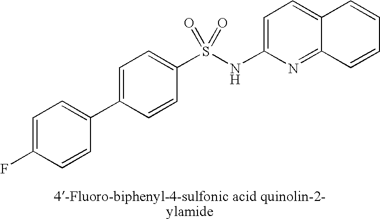 | F | (400 MHz, CDCl₃) δ: 8.02-8.10 (m, 2 H), 8.06 (d, 2 H), 7.88 (d, J=9.3 Hz, 1 H), 7.60-7.69 (m, 4 H) 7.50-7.58 (m, 2 H), 7.40-7.45 (m, 1 H), 7.35-7.40 (m, 1 H), 7.11-7.19 (m, 1 H), 6.88 (d, J=9.3 Hz, 1 H) | NA |
| 49 | NA | 82.6 | 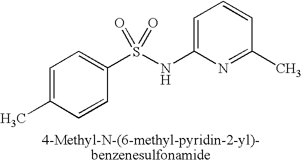 | F | (400 MHz, CDCl₃) δ: 7.80 (d, J=83 Hz, 2 H), 7.48 (dd, J=8.5, 7.4 Hz, 1 H), 7.24 (m, 2 H), 7.06 (d, J =8.6 Hz, 1 H), 6.62 (d, J =7.3 Hz, 1 H), 2.42 (s, 3 H), 2.37 (s, 3 H) | 263.0855 |
| 50 | NA | 10.1 | 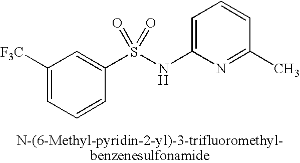 | F | (400 MHz, CDCl₃) δ: 8.22 (s, 1 H), 8.14 (d, J=8.1 Hz, 1 H), 7.75 (d, J=7.8 Hz, 1 H), 7.50-7.64 (m, 2 H), 7.06 (d, J=8.8 Hz, 1 H), 6.57 (d, J=7.3 Hz, 1 H), 2.49 (s, 3 H) | 317.0563 |
| 51 | NA | 10.2 | 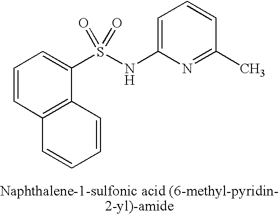 | F | (400 MHz, CDCl₃) δ: 8.88 (d, J=8.6 Hz, 1 H), 8.33 (dd, J=7.3, 1.0 Hz, 1 H), 7.99 (d, J=8.1 Hz, 1 H), 7.88 (d, J=8.1 Hz, 1 H), 7.63 (m, 1 H), 7.40-7.57 (m, 3 H), 6.98 (d, J=8.8 Hz, 1 H), 6.48 (d, J=7.3 Hz, 1 H), 2.41 (s, 3 H) | 299.0849 |
| 52 | NA | 32 | 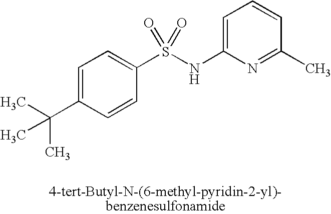 | F | (400 MHz, CDCl₃) δ: 7.84 (m, 2 H), 741-7.53 (m, 3 H), 7.11 (d, J=8.6 Hz, 1 H), 6.60 (d, J=7.3 Hz, 1 H), 2.45 (s, 3 H), 1.29 (s, 9 H) | 305.1325 |
| 53 | NA | 10.1 | 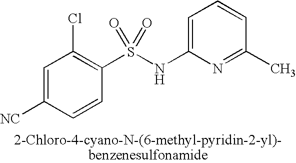 | F | (400 MHz, CDCl₃) δ: 8.33 (d, J=8.3 Hz, 1 H), 7.73 (d, J =1.5 Hz, 1 H), 7.66 (dd, J=8.2, 1.6 Hz, 1 H), 7.58 (dd, J=8.8, 7.1 Hz, 1 H), 6.84 (d, J=8.6 Hz, 1 H), 6.55 (d, J=7.1 Hz, 1 H), 2.48 (s, 3 H) | 308.0266 |
| 54 | NA | 1.0 | 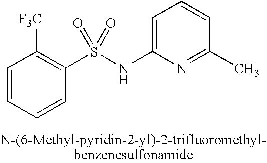 | F | (400 MHz, CDCl₃), δ: 8.37 (d, J=7.6 Hz, 1 H), 7.80 (m, 1 H), 7.57-7.70 (m, 2 H), 7.51 (dd, J=8.7, 7.2 Hz, 1 H), 6.84 (d, J=8.8 Hz, 1 H), 6.52 (d, J=7.3 Hz, 1 H), 2.44 (s, 3 H) | 317.0570 |
| 55 | NA | 7.4 | 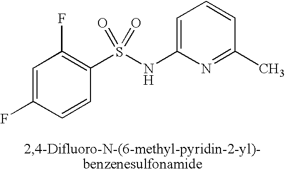 | F | (400 MHz, CDCl₃), δ: 8.02 (m, 1 H), 7.54 (dd, J=8.8, 7.1 Hz, 1 H), 6.95 (m, 1 H), 6.79-6.90 (m, 2 H), 6.55 (d, J=7.1 Hz, 1 H), 2.46 (s, 3 H) | 285.0509 |
| 56 | 47.6 | 66.8 | 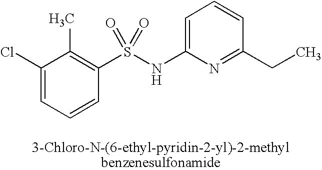 | F | (400 MHz, CDCl₃) δ: 8.02 (dd, J=8.0, 1.1 Hz, 1 H), 7.42-7.59 (m, 2 H), 7.20 (m, 1 H), 6.85 (d, J=8.8 Hz, 1 H), 6.51 (d, J=7.1 Hz, 1 H), 2.76 (s, 3 H), 2.72 (q, J=7.7 Hz, 2 H), 1.29 (t, J=7.6 Hz, 3 H) | 311.0627 |
| 57 | NA | 4.5 | 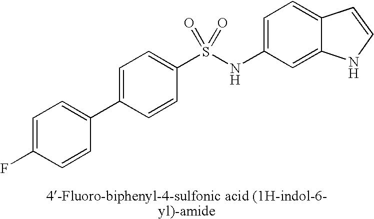 | F | (400 MHz, CDCl₃) δ: 6.43 (s, 1 H), 6.50 (m, 1 H), 6.65 (t, J=8.2, 1.9 Hz, 1 H), 7.14 (m, 2 H), 7.22 (d, J=2.53 Hz, 2 H), 7.38 (s, 1 H), 7.47 (s, 1 H), 7.50 (m, 1 H), 7.52 (d, J=3.5 Hz, 2 H), 7.55 (d, J=8.6 Hz, 2 H), 7.75 (d, J=8.34 Hz, 2 H) | 367.1 |
| 58 | NA | 4.0 | 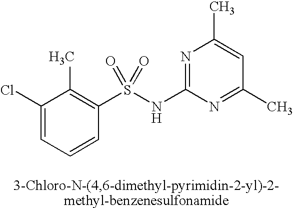 | F | (400 MHz, CDCl₃) δ: 8.76 (br s, 1 H), 8.24 (dd, J =8.1, 1.0 Hz, 1 H), 7.55 (dd, J=8.1, 1.0 Hz, 1 H), 7.26-7.31 (m, 1 H), 6.59 (s, 1 H), 2.71 (s, 3 H), 2.29 (s, 6 H) | 312.0558 |
| 59 | NA | 3.8 | 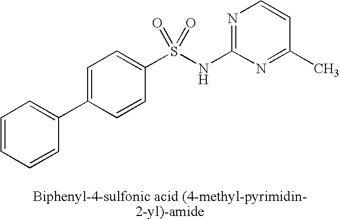 | F | (400 MHz, DMSO-d₅) δ: 11.77 (br s,1 H), 8.32 (d, J=5.3 Hz, 1 H), 8.04 (d, J=8.3 Hz,2 H), 7.85 (d, J=8.3 Hz, 2 H), 7.71 (d, J=7.3 Hz, 2 H), 7.38-7.52 (m, 3 H), 6.91 (d, J=5.1 Hz, 1 H), 2.32 (s, 3 H) | 326.0974 |
| 60 | NA | 31.9 | 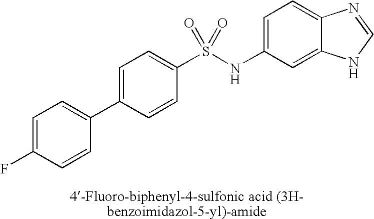 | F | (400 MHz, CDCl₃) δ: 6.78 (d, J=8.59 Hz, 1 H), 7.02 (s, 1 H), 7.14 (m, 2 H), 7.49 (m, 2 H), 7.64 (m, 3H), 7.99 (m, 2 H), 8.29 (s, 1 H) | 368.0 |
| 61 | 2.3 | 98.6 | 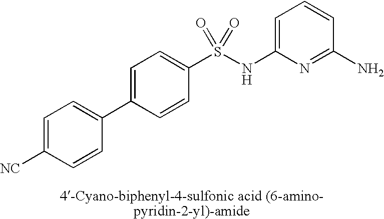 | F | (400 MHz, MeOD) δ: 6.22 (d, J=7.8 Hz, 1 H), 6.28 (d, J=8.3 Hz, 1 H), 7.45 (t, J=8.2 Hz, 1 H), 7.70-7.81 (m, 6 H), 7.90 (d, J =8.3 Hz, 2 H) | 351.1 |
| 62 | NA | 1.3 | 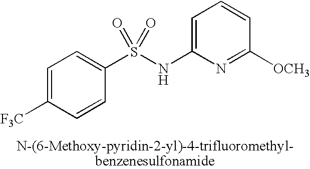 | F | (400 MHz, CDCl₃) δ ppm 3.71 (s, 3 H) 6.42 (d, J=8.08 Hz, 1 H) 6.77 (d, J=7.83 Hz, 1 H) 7.49 (t, J=7.96 Hz, 1 H) 7.74 (d, J=8.59 Hz, 2 H) 8.10 (d, J=8.08 Hz, 2 H) | 333.1 |
| 63 | NA | 32.7 | 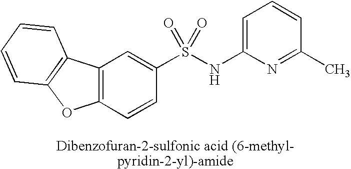 | F | (400 MHz, CDCl₃) δ: 8.58 (d, J=2.0 Hz, 1 H) 8.03 (dd, J=8.7, 1.9 Hz, 1 H) 7.97 (d, J=7.8 Hz, 1 H) 7.57-7.62 (m, 2 H) 7.45-7.54 (m, 2 H) 7.39 (t, J=7.5 Hz, 1 H) 7.01 (d, J=8.6 Hz, 1 H) 6.59 (d, J=7.6 Hz, 1 H) 2.39 (s, 3 H) | 339.0792 |
| 64 | NA | 8.9 | 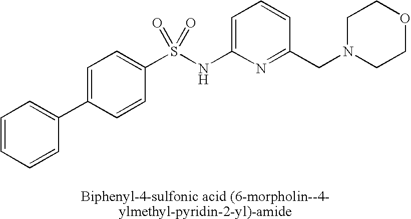 | F | (400 MHz, CDCl₃) δ: 11.21 (s, 1 H) 8.01 (d, J=8.3 Hz, 2 H) 7.65 (d, J=8.3 Hz, 2 H) 7.35-7.59 (m, 6 H) 6.98 (d, J=8.8 Hz, 1 H) 6.55 (d, J=7.1 Hz, 1 H) 3.80 (m, 4 H) 3.52 (s, 2 H) 2.51 (m, 4 H) | 410.1520 |
| 65 | 18.3 | 59.4 | 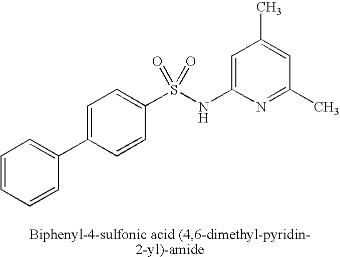 | F | (400 MHz, CDCl₃) δ: 8.00 (m, 2 H), 7.64 (m, 2 H), 7.55 (m, 2 H), 7.34-7.47 (m, 3 H), 6.95 (s, 1 H), 6.40 (s, 1 H), 2.44 (s, 3 H), 2.26 (s, 3 H) | 339.1157 |
| 66 | NA | 33.8 | 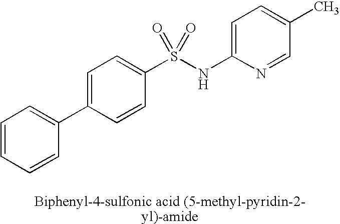 | F | (400 MHz, CDCl₃) δ: 8.13 (s, 1 H), 7.97 (d, J=8.3 Hz, 2 H), 7.64 (d, J=8.6 Hz, 2 H), 7.50-7.58 (m, 3 H), 7.36-7.48 (m, 4 H), 2.22 (s, 3 H) | 325.0997 |
| 67 | NA | 49.6 | 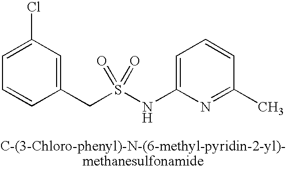 | F | (400 MHz, CDCl₃) δ: 10.35 (s, J=16.2 Hz, 1 H), 7.36-7.48 (m, 2 H), 7.23-7.31 (m, 1 H), 7.14-7.23 (m, 2 H), 6.71 (d, J=8.8 Hz, 1 H), 6.41 (d, J=7.3 Hz, 1 H), 4.33-4.41 (m, 2 H) 2.25 (s, 3 H) | 297.0451 |
| 68 | 8.3 | 100 | 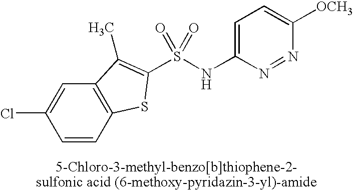 | F | (400 MHz, CDCl₃) δ: 7.66-7.78 (m, 2 H), 7.39 (dd, J =8.6, 2.0 Hz, 1 H), 7.25 (d, J=9.8 Hz, 1 H), 7.07 (d, J=9.6 Hz, 1 H), 3.89 (s, 3 H), 2.68 (s, 3 H) | 370.0 |
| 69 | 1.1 | 100 | 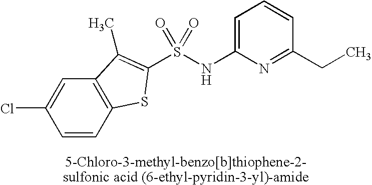 | F | (400 MHz, CDCl₃) δ: 11.43 (br s, 1 H), 7.64-7.76 (m, 2 H), 7.56 (dd, J=8.8, 7.3 Hz, 1 H), 7.38 (dd, J=8.6, 2.0 Hz, 1 H), 6.95 (d, J =8.8 Hz, 1 H), 6.53 (d, J =7.3 Hz, 1 H), 2.74 (q, J =7.6 Hz, 2 H), 2.68 (s, 3 H) 1.31 (t, J=7.6 Hz, 3 H) | 367.0 |
| 70 | 216 | 78.7 | 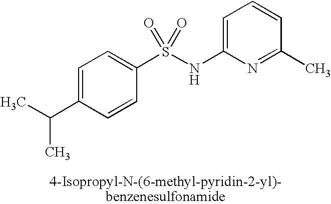 | F | (400 MHz, CDCl₃) δ: 9.73 (br s, 1 H), 7.83 (d, J=8.6 Hz, 2 H), 7.48 (dd, J=8.6, 7.3 Hz, 1 H), 7.29 (d, J =8.3 Hz, 2 H), 7.04 (d, J =8.6 Hz, 1 H), 6.61 (d, J =7.3 Hz, 1 H), 2.92 (m, 1 H), 2.41 (s, 3 H), 1.22 (d, J=71 Hz, 6 H) | 291.1158 |
| 71 | 34.6 | 66 | 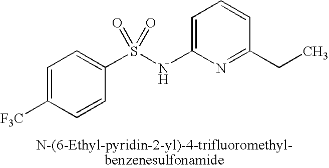 | F | (400 MHz, CDCl₃) δ: 11.17 (br s, 1 H), 8.06 (d, J =8.1 Hz, 2 H), 7.70 (d, J =8.3 Hz, 2 H), 7.55 (dd, J =8.6, 7.3 Hz, 1 H), 6.94 (d, J=8.8 Hz, 1 H), 6.55 (d, J=7.3 Hz, 1 H), 2.73 (q, J=7.6 Hz, 2 H), 1.29 (t, J=7.6 Hz, 3 H) | 331.0716 |
| 72 | 30.9 | 74.6 | 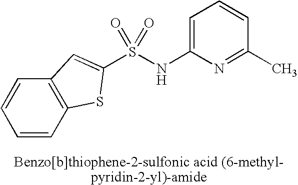 | F | (400 MHz, CDCl₃) δ: 7.90 (s, 1 H), 7.71-7.86 (m, 2 H), 7.57 (dd, J=8.8, 7.3 Hz, 1 H), 7.32-7.47 (m, 2 H), 7.21 (d, J=8.8 Hz, 1 H), 6.57 (d, J=7.3 Hz, 1 H), 2.52 (s, 3 H) | 305.0 |
| 73 | NA | 17.3 | 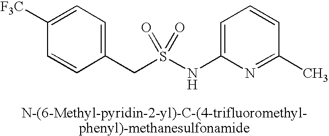 | F | (400 MHz, CDCl₃) δ: 7.51 (m, 4 H), 7.43 (dd, J=8.7, 7.2 Hz, 1 H), 6.68 (d, J =8.8 Hz, 1 H), 6.39 (d, J =7.1 Hz, 1 H), 4.45 (s, 2 H), 2.20 (s, 3 H) | 331.1 |
| 74 | NA | 28.9 | 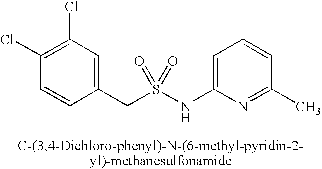 | F | (400 MHz, CDCl₃) δ: 7.42-7.51 (m, 2 H), 7.31 (m, 1 H), 7.21-7.25 (m, 1 H), 6.72 (d, J=8.8 Hz, 1 H), 6.44 (d, J=7.3 Hz, 1 H), 4.33 (s, 2 H), 2.27 (s, 3 H) | 333.0 |
| 75 | NA | 19.2 | 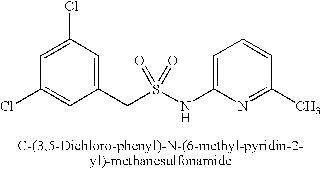 | F | (400 MHz, CDCl₃), δ: 7.47 (dd, J=8.8, 7.3 Hz, 1 H), 7.29 (d, J=1.8 Hz, 2 H), 7.22 (t, J=1.9 Hz, 1 H), 6.71 (d, J=8.6 Hz, 1 H), 6.43 (d, J=7.1 Hz, 1 H), 4.31 (s, 2 H), 2.28 s, 3 H) | 333.0 |
| 76 | NA | 15.6 | 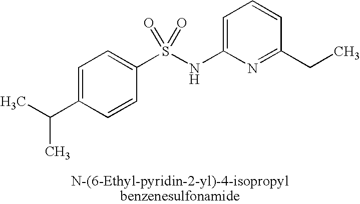 | F | (400 MHz, CDCl₃) δ: 9.75 (br s, 1 H), 7.84 (d, J=8.6 Hz, 2 H), 7.49 (dd, J=8.5, 7.5 Hz, 1 H), 7.29 (d, J =8.3 Hz, 2 H), 6.98 (d, J =8.6 Hz, 1 H), 6.60 (d, J =7.3 Hz, 1 H), 2.92 (m, 1 H), 2.67 (q, J=7.6 Hz, 2 H), 1.12-1.29 (m, 9 H) | 305.1309 |
| 77 | NA | 35.4 | 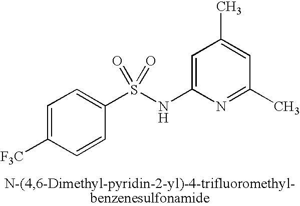 | F | NA | 331.0738 |
| 78 | NA | 8.2 | 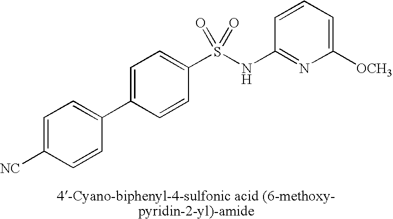 | F | (400 MHz, CDCl₃) δ: 3.74 (s, 3 H), 6.40 (d, J=8.1 Hz, 1 H), 6.81 (d, J=7.8 Hz, 1 H), 7.49 (t, J=8.0 Hz, 1 H), 7.68 (d, J=7.6 Hz, 1 H), 7.75 (m, 2 H), 8.09 (d, J=8.3 Hz, 2 H) | 366.1 |
| 79 | NA | 8.1 | 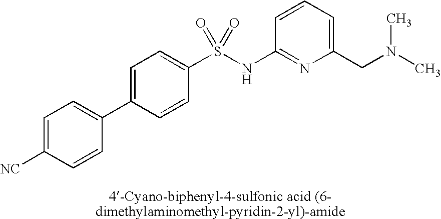 | F | (400 MHz, CDCl₃) δ: 8.07 (d, J=8.3 Hz, 2 H) 7.72-7.76 (m, 2 H) 7.62-7.66 (m, 4 H) 7.52 (dd, J=8.8, 7.1 Hz, 1 H) 7.02 (d, J=8.6 Hz, 1 H) 6.49 (d, J=7.1 Hz, 1 H) 3.47 (s, 2 H) 2.33 (s, 6 H) | 393.1377 |
| 80 | NA | 8 | 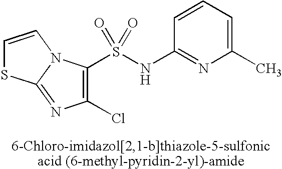 | F | (400 MHz, DMSO-d₆) δ: 13.60 (br s, 1 H), 7.91 (m, 1 H), 7.77 (m, 1 H), 7.55 (m, 1 H), 7.09 (m, 1 H), 6.71 (m, 1 H), 2.33 (s, 3 H) | 329.0 |
| 81 | NA | 23.4 | 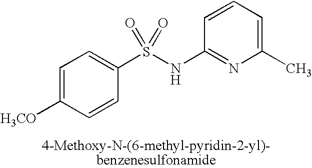 | F | (400 MHz, CDCl₃) δ: 7.90 (d, J=7.6 Hz, 2 H), 7.69 (m, 1 H), 7.34 (d, J=8.1 Hz, 1.H), 6.94 (d, J=7.8 Hz, 2 H), 6.81 (d, J=6.8 Hz, 1 H), 3.83 (s, 3 H), 2.52 (s, 3 H) | 279.0797 |
| 82 | NA | 53.4 | 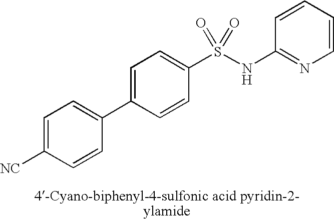 | F | (400 MHz, DMSO-d₆) δ: 12.30 (br s, 1 H), 7.84-8.04 (m, 9 H), 7.74 (m, 1 H), 7.21 (d, J=8.6 Hz, 1 H), 6.85 (t, J=6.3 Hz, 1 H) | 336.1 |
| 83 | NA | 11.1 | 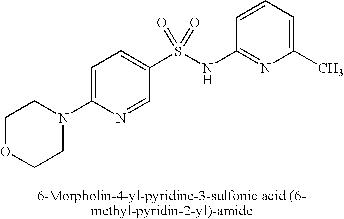 | F | (400 MHz, CDCl₃), δ: 9.45 (br s, 1 H), 8.66 (d, J=2.5 Hz, 1 H), 7.91 (dd, J=9.1, 2.5 Hz, 1 H), 7.41-7.56 (m, 1 H), 7.01 (d, J=8.6 Hz, 1 H), 6.63 (d, J=7.3 Hz, 1 H), 6.56 (d, J=9.1 Hz, 1 H), 3.70-3.83 (m, 4 H), 3.54-3.66 (m, 4 H), 2.41 (s, 3 H) | 335.0 |
| 84 | NA | 26.4 | 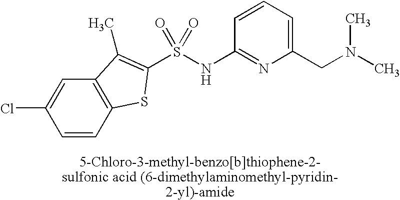 | F | (400 MHz, CDCl₃) δ: 7.72 (d, J=2.0 Hz, 1 H) 7.67 (d, J=8.6 Hz, 1 H) 7.54 (dd, J=8.8, 7.1 Hz, 1 H) 7.36 (dd, J=8.6, 1.8 Hz, 1 H) 7.04 (d, J=8.8 Hz, 1 H) 6.46 (d, J=7.1 Hz, 1 H) 3.47 (s, 2 H) 2.69 (s, 3 H) 2.33 (s, 6 H) | 396.0597 |
| 85 | 6.6 | 100 | 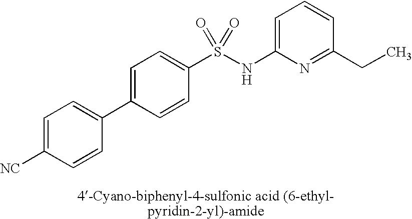 | F | (400 MHz, CDCl₃) δ: 8.10 (d, J=7.8 Hz, 2 H), 7.65-7.78 (m, 7 H), 7.33 (d, J =8.6 Hz, 1 H), 6.80 (d, J =7.1 Hz, 1 H), 2.82 (q, J =7.3 Hz, 2 H), 1.34 (t, J =7.3 Hz, 3 H) | 364.1102 |
| 86 | 14.3 | 100 | 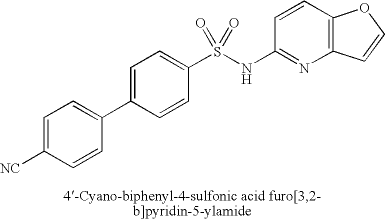 | F | (400 MHz, CDCl₃) δ: 3.74 (s, 3 H), 6.40 (d, J=8.1 Hz, 1 H), 6.81 (d, J=7.8 Hz, 1 H), 7.49 (t, J=8.0 Hz, 1 H), 7.68 (t, J=7.58 Hz, 4 H), 7.75 (m, 2 H), 8.09 (d, J=8.3 Hz, 2 H) | 376.0 |
| 87 | 3.6 | 100 | 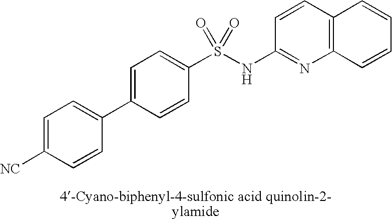 | F | (400 MHz, CDCl₃) δ: 7.20-7.34 (m, 2 H), 7.46 (t, J =7.6 Hz, 1 H), 7.58-7.85 (m, 8 H), 8.07 (d, J=9.4 Hz, 1 H), 8.13 (d, J=8.1 Hz, 2 H) | 386 |
| 88 | NA | 7.7 | 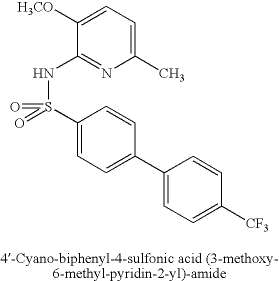 | F | (400 MHz, CDCl₃) δ: 8.27 (s, 2H), 7.63-7.84 (m, 6 H), 6.91 (d, J=7.8 Hz, 2 H), 3.81 (s, 3H), 2.38 (s, 3H) | 380.1 |
| 89 | NA | 3.7 | 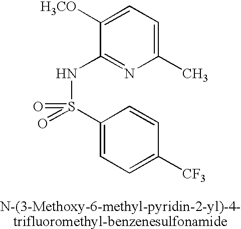 | F | (400 MHz, CDCl₃) δ: 8.28-8.26 (m, 1H), 7.75-7.73 (m, 1H), 6.93-6.91 (m, 1H), 6.86-6.84 (m, 1H), 6.61-6.59 (m, 1H), 6.45-6.43 (m, 1H), 3.81 (s, 3H), 2.35 (s, 3H) | 347.0660 |
| 90 | NA | 24.8 | 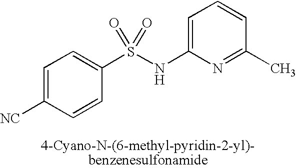 | F | (400 MHz, CDCl₃) δ: 8.06 (d, J=8.1 Hz, 2 H), 7.75 (d, J=8.4 Hz, 2 H), 7.59 (m, 1 H), 7.01 (d, J=8.9 Hz, 1 H), 6.60 (m, 1 H), 2.48 (s, 3 H) | 274.0634 |
| 91 | 2.3 | 100 | 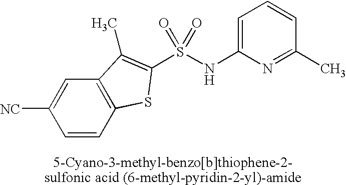 | F | (400 MHz, DMSO-d₆) δ: 13.58 (br s, 1 H), 8.43 (s, 1 H), 8.21 (d, J=8.3 Hz, 1 H), 7.82 (dd, J=8.3, 1.3 Hz, 1 H), 7.72 (m, 1 H), 7.16 (m, 1 H), 6.68 (br d, J=7.3 Hz, 1 H), 2.63 (s, 3 H), 2.34 (s, 3 H) | 344.0520 |
| 92 | NA | 29.8 | 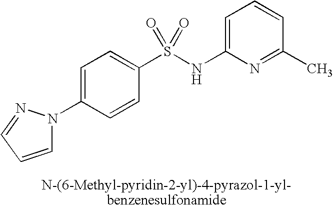 | F | (400 MHz, CDCl₃) δ: 8.01 (m, 2 H), 7.96 (d, J =2.5 Hz, 1 H), 7.79 (m, 2 H), 7.73 (d, J=1.5 Hz, 1 H), 7.51 (dd, J=8.7, 7.5 Hz, 1 H), 7.04 (d, J=8.8 Hz, 1 H), 6.60 (d, J=7.3 Hz, 1 H), 6.49 (m, 1 H), 2.43 (s, 3 H) | 315.0 |
| 93 | 42.3 | 70 | 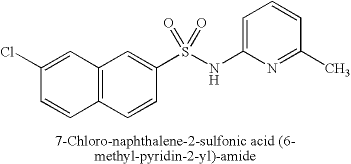 | F | (400 MHz, CDCl₃) δ: 8.40 (s, 1 H), 7.83-7.96 (m, 3 H), 7.79 (d, J=8.8 Hz, 1 H), 7.47-7.55 (m, 2 H), 7.05 (d, J=8.8 Hz, 1 H), 6.57 (d, J=7.3 Hz, 1 H), 2.44 (s, 3 H) | 333.0 |
| 94 | 32.8 | 76.3 | 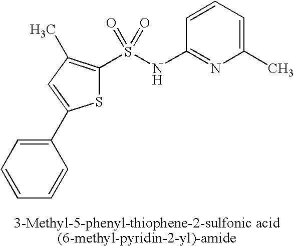 | F | (400 MHz, CDCl₃), δ: 7.86 (m, 2 H), 7.58 (dd, J=8.8, 7.3 Hz, 1 H), 7.35-7.49 (m, 3 H), 7.10 (d, J=8.8 Hz, 1 H), 6.58 (d, J=7.1 Hz, 1 H), 2.74 (s, 3 H) 2.51 (s, 3 H) | 346.0 |
| 95 | 4.4 | 100 | 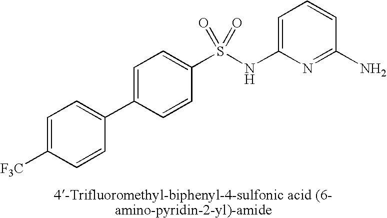 | F | (400 MHz, MeOD) δ: 4.74 (d, J=8.08 Hz, 2 H) 5.04 (d, J=8.08 Hz, 1 H) 6.06 (t, J=8.08 Hz, 1 H) 6.45-6.49 (m, 2 H) 6.50-6.54 (m, 2 H) 6.54-6.59 (m, 2 H) 6.73 (d, J=8.59 Hz, 2 H) | NA |
| 96 | 7 | 100 | 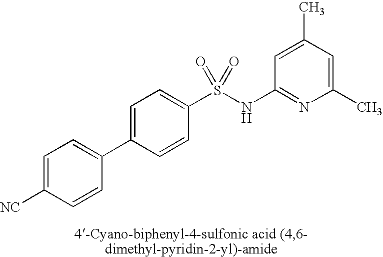 | F | (400 MHz, DMSO-d₆), δ: 12.97 (br s, 1 H), 7.84-7.97 (m, 8 H), 6.94 (s, 1 H), 6.48 (s, 1 H), 2.25 (s, 3 H), 2.19 (s, 3 H) | 364.1 |
| 97 | NA | 31.7 | 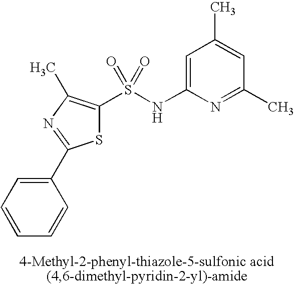 | F | (400 MHz, CDCl₃) δ: 7.79-7.91 (m, 2 H), 7.33-7.49 (m, 3 H), 6.95 (s, 1 H), 6.39 (s, 1 H), 2.75 (s, 3 H), 2.49 (s, 3 H), 2.28 (s, 3 H) | 360.1 |
| 98 | NA | 8.9 | 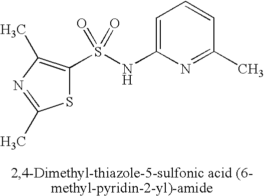 | F | (400 MHz, CDCl₃) δ: 7.57 (dd, J=8.8, 7.3 Hz, 1 H), 7.11 (d, J=8.8 Hz, 1 H), 6.56 (d, J=7.1 Hz, 1 H), 2.63 (s, 3 H), 2.62 (s, 3 H), 2.52 (s, 3 H) | 284.1 |
| 99 | NA | 15 | 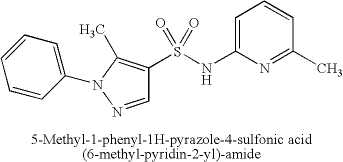 | F | (400 MHz, CDCl₃) δ: 7.86-8.01 (m, 1 H), 7.41-7.59 (m, 4 H), 733-7.41 (m, 2 H), 7.04 (d, J=6.6 Hz, 1 H), 6.67 (d, J=7.3 Hz, 1 H), 2.52 (s, 3 H), 2.44 (s, 3 H) | 329.1 |
| 100 | NA | 19.2 | 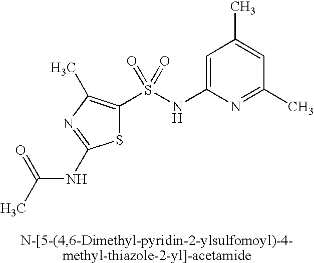 | F | (400 MHz, CDCl₃) δ: 6.97 (s, 1 H), 6.37 (s, 1 H), 4.56 (br s, 1 H), 2.52 (s, 3 H), 2.44 (s, 3 H), 2.27 (s, 3 H), 2.23 (s, 3 H) | 341.1 |
| 101 | NA | 32 | 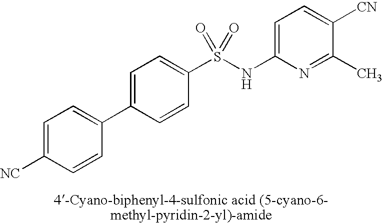 | F | (400 MHz, DMSO-d₆), δ: 7.90-8.11 (m, 10 H), 2.50 (s, 3 H) | 375.1 |
| 102 | <1 | 100 | 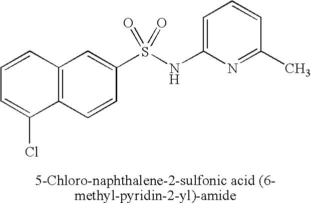 | F | NA | 332.9 |
| 103 | <1 | 100 | 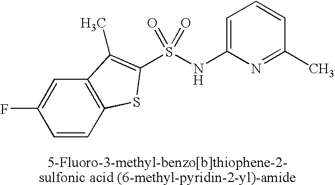 | F | NA | 337 |
| 104 | 23 | 89.6 | 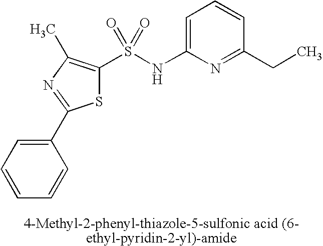 | F | (400 MHz, CDCl₃) δ: 7.86 (m, 2 H), 7.59 (dd, J =8.7, 7.2 Hz, 1 H), 7.35-7.48 (m, 3 H), 7.00 (d, J =8.8 Hz, 1 H), 6.57 (d, J =7.3 Hz, 1 H), 2.76 (m, 2 H), 2.73 (s, 3 H), 1.31 (t, J=7.6 Hz, 3 H) | 360.1 |
| 105 | NA | 16.9 | 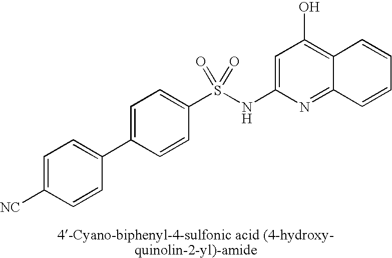 | F | (400 MHz, CDCl₃) δ: 8.11-8.14 (m, 2 H), 7.87 (d, J=8.1 Hz, 1 H), 7.81 (dd, J=8.5, 1.9 Hz, 4 H), 7.66-7.75 (m, 4 H), 7.37 (t, 1 H) 6.95 (s, 1 H) | 402.1 |
| 106 | NA | 5.4 | 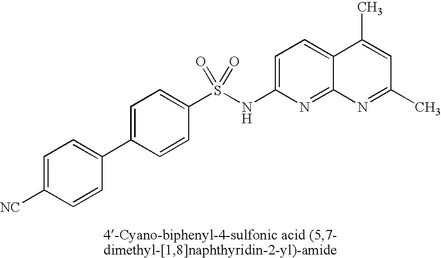 | F | (400 MHz, CDCl₃) δ: 8.56 (s, 1 H) 8.33 (d, J=7.8 Hz, 1 H) 7.88-7.94 (m, 1 H) 7.86 (s, 4 H) 7.44 (d, J=9.3 Hz, 1 H) 2.87 (s, 3 H) 2.81 (s, 3 H) | NA |
| 107 | <1 | 100 | 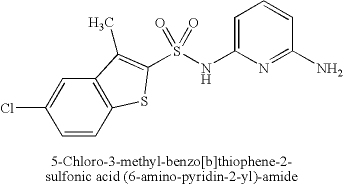 | F | (400 MHz, CDCl₃) δ: 7.63-7.69 (m, 2 H), 7.37-7.46 (m, 2 H), 6.88 (d, J=8.3 Hz, 1 H), 5.97 (d, J=8.1 Hz, 1 H), 2.61 (s, 3 H) | 354.0 |
| 108 | <1 | 100 | 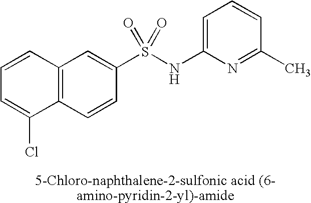 | F | (400 MHz, DMSO-d₆) δ: 8.62 (br s, 1 H), 8.28 (d, J=9.0Hz, 1 H), 8.20 (d, J=8.1 Hz, 1 H), 8.03 (d, J=8.3 Hz, 1 H), 7.64 (d, J=7.3 Hz, 1 H), 7.62 (t, J=8.0 Hz, 1 H), 7.28 (t, J=8.1 Hz, 1 H), 6.46 (bs, 2 H), 6.19 (d, J=8.3 Hz, 1 H), 5.88 (d, J=8.1 Hz, 1 H) | 334.2 |
| 109 | 270 | 57.2 | 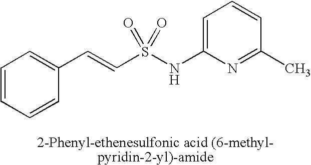 | F | (400 MHz, CDCl₃) δ: 7.69 (dd, J=8.6, 7.6 Hz, 1 H) 7.56 (d, J=15.4 Hz, 1 H) 7.39-7.45 (m, 2 H) 7.26-7.37 (m, 4 H) 6.87 (d, J=15.4 Hz, 1 H) 6.78 (d, J=7.6 Hz, 1 H) 2.49 (s, 3 H) | NA |
| 110 | NA | 8.1 | 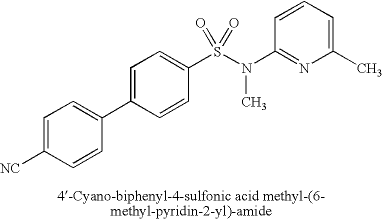 | G | (400 MHz, CD₃CN), δ: 7.83-7.75 (m, 9 H), 7.67 (d, J=8.3 Hz,2 H), 7.50-7.47 (m, 1H) , 7.32 (d, J =8.1 Hz, 1 H), 3.30 (m, 3 H), 2.57 (s, 3 H) | 364.1 |
| 111 | 1.7 | 100 | 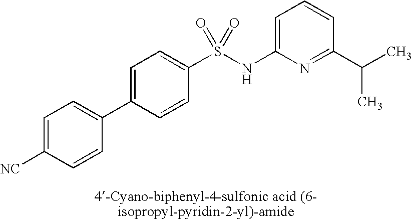 | H | (400 MHz, CD₃CN) δ: 9.45 (br s, 1 H), 8.05 (dd, J =6.6, 1.8 Hz, 1 H), 7.90-7.78 (m, 6 H), 7.62 (t, J =8.4 Hz, 1 H), 6.98 (d, J =7.8 Hz, 1 H), 6.79 (d, J =7.6 Hz, 1 H), 2.90-2.86 (m, 1 H), 1.18 (d, J=8.7 Hz, 6 H) | 364.1 |
| 112 | 1.9 | 100 | 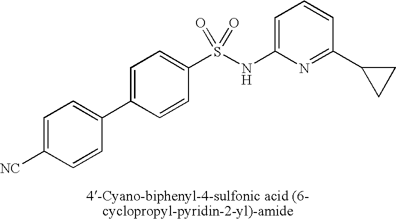 | I | (400 MHz, CDCl₃) δ: 8.04 (d, J=8.5, 1 H), 7.76 (d, J=8.3 Hz, 1 H), 7.67 (d, J=8.5 Hz, 1 H), 7.47 (t, J=7.6 Hz, 1 H), 6.94 (d, J=8.4 Hz, 1 H), 6.65 (d, J=7.6 Hz, 1 H), 1.93-1.87 (m, 1 H), 1.01-0.97 (m, 2 H), 0.88-0.85 (m, 1 H) | 376.1112 |
| 113 | NA | 100 | 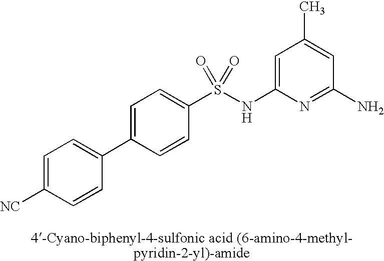 | J | (400 MHz, DMSO-D6, D₂O) δ 2.04 (s, 3 H) 5.72 (s, 1 H) 6.09 (s, 1 H) 7.83-7.68 (m, 2 H) 7.89-7.96 (m, J=8.51, 8.51, 8.51 Hz, 6 H) | 365.1 |
| 114 | NA | 11.3 | 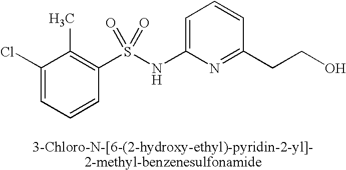 | K | (400 MHz, CDCl₃) δ: 8.07 (d, J=7.8 Hz, 1 H), 7.41-7.65 (m, 2 H), 7.20-7.26 (m, 1 H), 7.01 (d, J=8.8 Hz, 1 H), 6.61 (d, J=7.3 Hz, 1 H), 3.98 (t, J=5.4 Hz, 2 H), 2.93 (t, J=5.6 Hz, 2 H), 2.77 (s, 3 H) | 327.0573 |
| 115 | 4.8 | 93.6 | 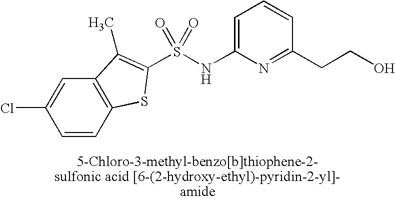 | L | (400 MHz, DMS0-d₆) δ: 8.02 (d, J=8.6 Hz, 1 H), 7.92 (m, 1 H), 7.72 (m, 1 H), 7.50 (dd, J=8.6, 2.0 Hz, 1 H), 7.15 (br s, 1 H), 6.71 (d, J=6.8 Hz, 1 H), 4.75 (br s, 1 H), 3.64 (m, 2 H), 2.76 (1, J=5.9 Hz, 2 H), 2.58 (s, 3 H) | 384.0 |
| 116 | NA | 37.2 | 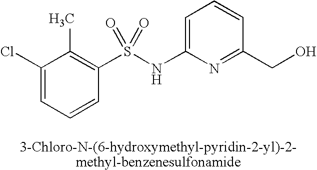 | L | (400 MHz, DMSO-d₆) δ: 7.97 (br s, 1 H); 7.50-7.80 (m, 2 H), 7.37 (br s, 1 H), 7.04 (brs, 1 H), 6.74 (br s, 1 H), 5.15-5.70 (m, 1 H), 4.20-4.50 (m, 2 H), 2.64 (s, 3H) | 313.0400 |
| 117 | 26.2 | 84.8 | 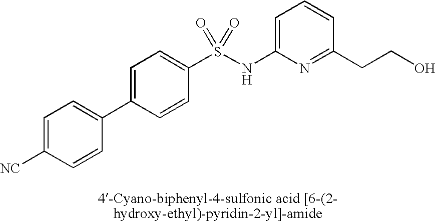 | L | (400 MHz, CDCl₃) δ: 8.08 (d, J=8.3 Hz, 2 H), 7.74 (m, 2 H), 7.63-7.68 (m, 4 H), 7.55 (dd, J=8.6, 7.3 Hz, 1 H), 7.11 (d, J=8.6 Hz, 1 H), 6.67 (d, J=47.3 Hz, 1 H), 4.00 (t, J=5.4 Hz, 2 H), 2.91 (t, J=5.4 Hz, 2 H), 1.24 (s, 1 H) | 380.0 |
| 118 | 2.5 | 100 | 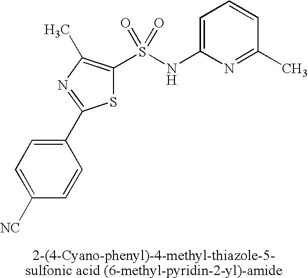 | M | (400 MHz, CDCl₃) δ: 7.98 (d, J=8.3 Hz, 2 H), 7.70 (d, J=8.3 Hz, 2 H), 7.61 (dd, J=8.7, 7.2 Hz, 1 H), 7.11 (d, J=8.8 Hz, 1 H), 6.59 (d, J=7.3 Hz, 1 H), 2.74 (s, 3 H), 2.53 (s, 3 H) | 371.2 |
| 119 | 3.1 | 95.0 | 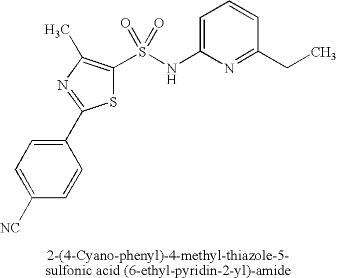 | M | (400 MHz, CDCl₃) δ: 11.64 (br s, 1 H), 7.98 (d, J=8.6 Hz, 2 H), 7.70 (d, J=8.6 Hz, 2 H), 7.62 (dd, J=9.0, 7.2 Hz, 1 H), 7.01 (d, J =8.6 Hz, 1 H), 6.58 (d, J =7.3 Hz, 1 H), 2.78 (q, J =7.6 Hz, 2 H), 2.73 (s, 3 H), 1.32 (t, J=7.6 Hz, 3 H) | NA |
| 120 | NA | 71.7 | 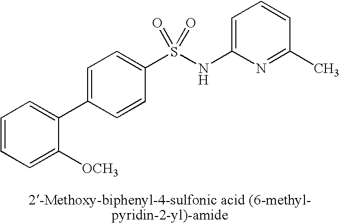 | N | NA | 355 |
| 121 | NA | 61.4 | 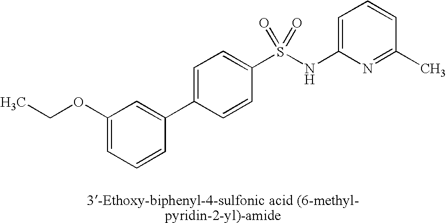 | N | NA | 369.1 |
| 122 | NA | 85.7 | 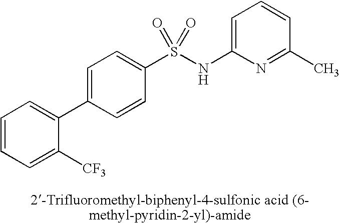 | N | NA | 393.1 |
| 123 | NA | 89.9 | 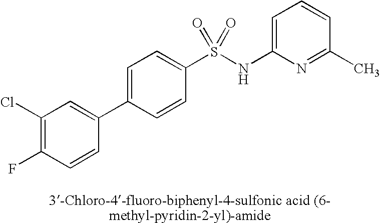 | N | NA | 377 |
| 124 | NA | 84 | 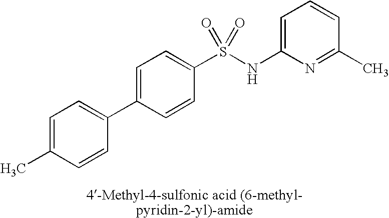 | N | NA | 339.1 |
| 125 | NA | 87.8 | 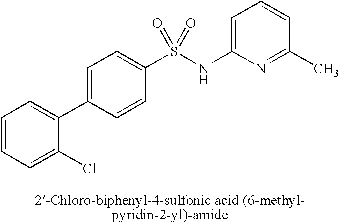 | N | NA | 359 |
| 126 | NA | 77.6 | 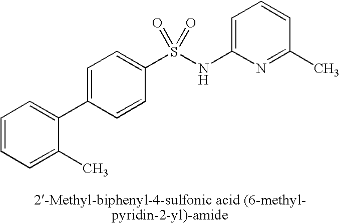 | N | NA | 339.1 |
| 127 | NA | 100 | 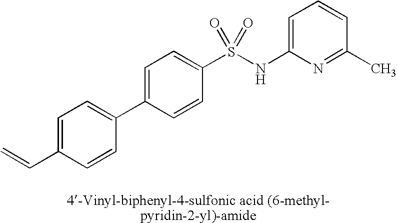 | N | NA | 351 1 |
| 128 | 19.7 | 86.8 | 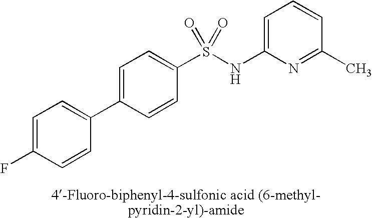 | N | NA | 343 |
| 129 | NA | 86.3 | 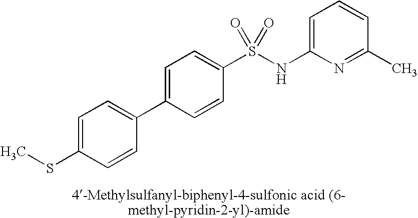 | N | NA | 371 |
| 130 | NA | 78.9 | 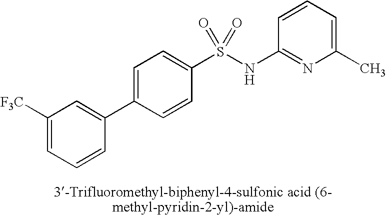 | N | NA | 393.1 |
| 131 | NA | 100 | 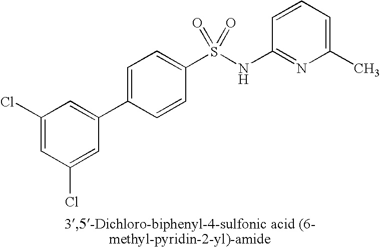 | N | NA | 392.9 |
| 132 | NA | 61.7 | 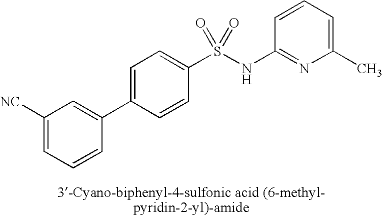 | N | NA | 350 |
| 133 | 24.7 | 84.8 | 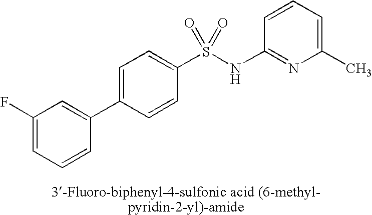 | N | NA | 343 |
| 134 | NA | 83.5 | 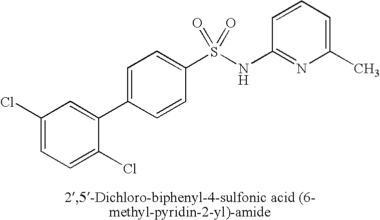 | N | NA | 392.9 |
| 135 | NA | 49.1 | 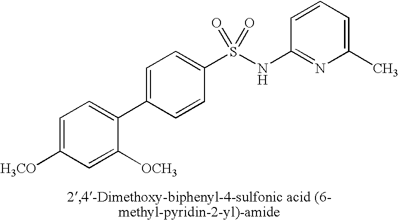 | N | NA | 385 |
| 136 | NA | 49.1 | 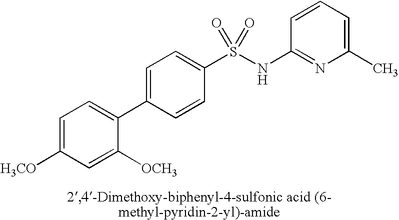 | N | NA | 385 |
| 137 | NA | 79 | 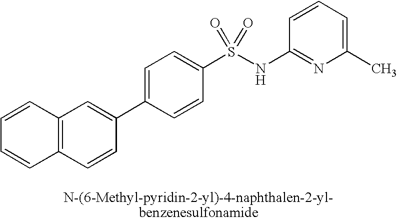 | N | NA | 375.1 |
| 138 | NA | 91.2 | 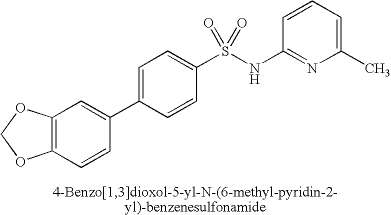 | N | NA | 368.9 |
| 139 | NA | 37.9 | 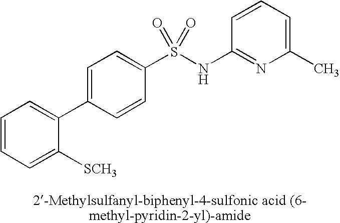 | N | NA | 371 |
| 140 | NA | 91% | 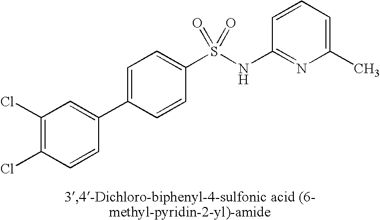 | N | NA | 392.9 |
| 141 | NA | 83.8 | 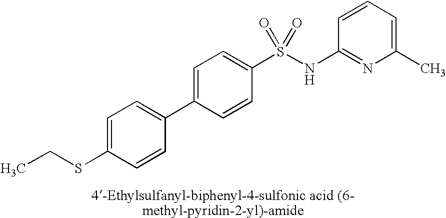 | N | NA | 385 |
| 142 | NA | 84.5 | 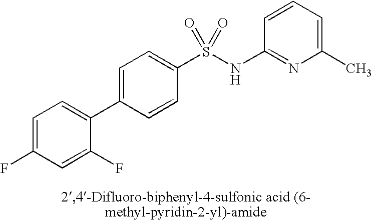 | N | NA | 361 |
| 143 | NA | 84.5 | 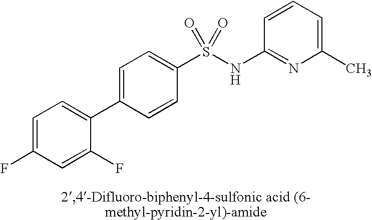 | N | NA | 381 |
| 144 | NA | 86.8 | 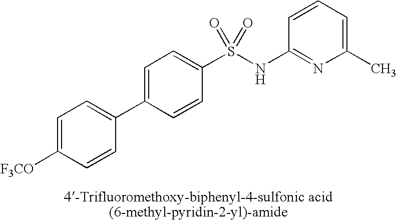 | N | NA | 409 |
| 145 | NA | 80.6 | 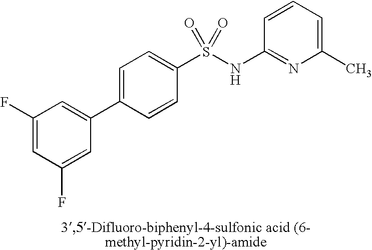 | N | NA | 361 |
| 146 | NA | 46.5 | 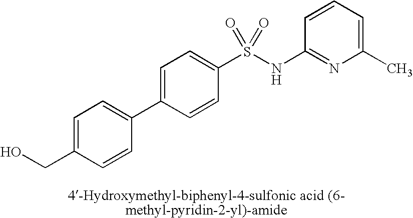 | N | NA | 355 |
| 147 | NA | 29.5 | 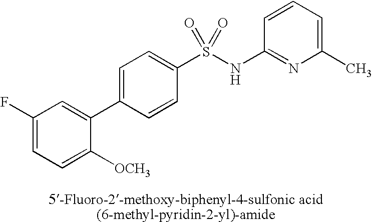 | N | NA | 373 |
| 148 | NA | 57.2 | 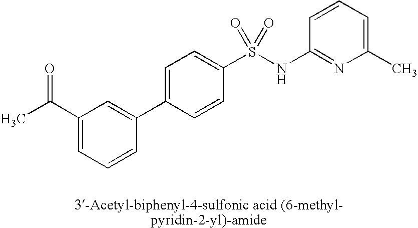 | N | NA | 367 |
| 149 | NA | 20.4 | 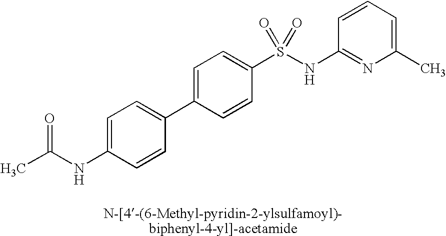 | N | NA | 382 |
| 150 | NA | 79.3 | 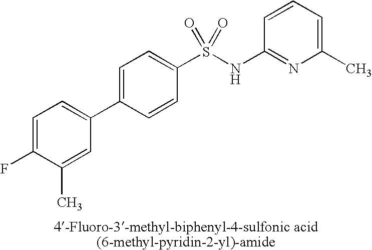 | N | NA | 357 |
| 151 | NA | 59.8 | 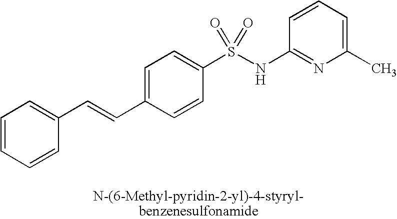 | N | NA | 351.1 |
| 152 | NA | 72.7 | 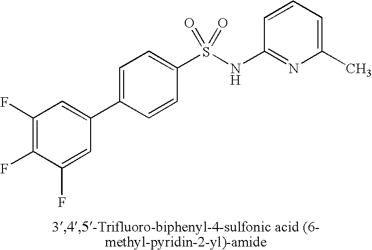 | N | NA | 379 |
| 153 | NA | 75.3 | 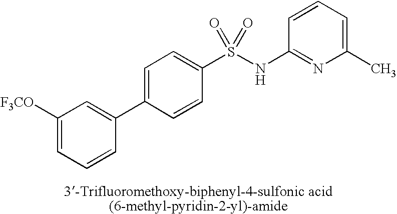 | N | NA | 409 |
| 154 | NA | 436 | 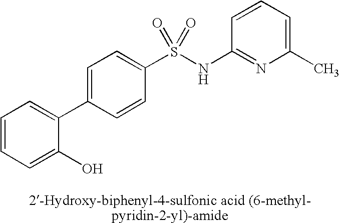 | N | NA | 341 |
| 155 | NA | 83.3 | 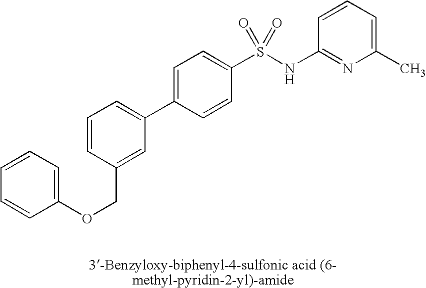 | N | NA | 431 |
| 156 | NA | 65.6 | 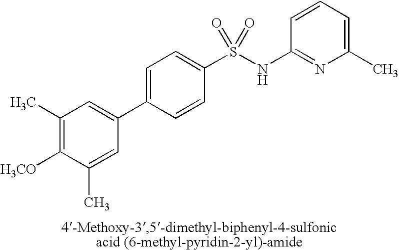 | N | NA | 383 |
| 157 | NA | 100 | 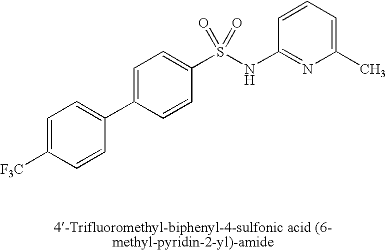 | N | NA | 392.9 |
| 158 | NA | 71 | 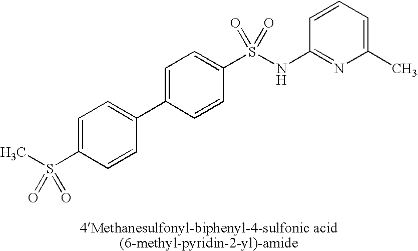 | N | NA | 403 |
| 159 | NA | 90.4 | 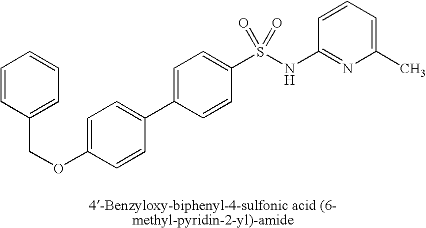 | N | NA | 431 |
| 160 | 36.2 | 66.8 | 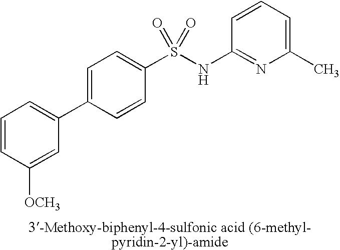 | N | NA | 355 |
| 161 | 22.5 | 90.3 | 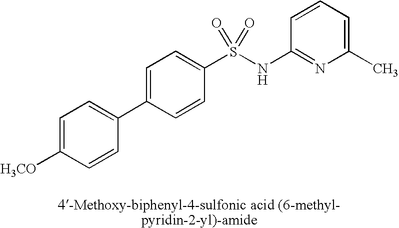 | N | NA | 355.1 |
| 162 | 21 | 89 | 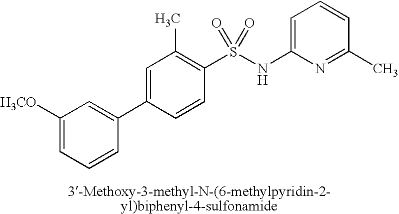 | N | NA | 369.1 |
| 163 | 8 | 97 | 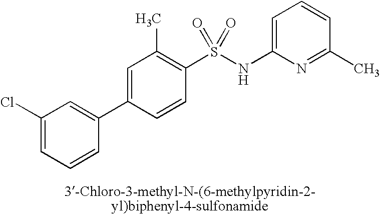 | N | NA | 373 |
| 164 | 13 | 93 | 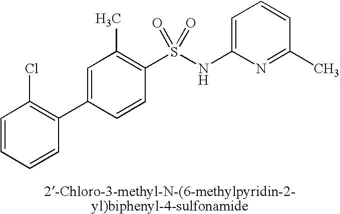 | N | NA | 373 |
| 165 | 43 | 71 | 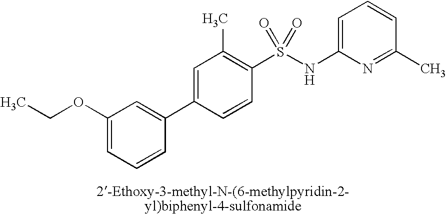 | N | NA | 383 |
| 166 | 12 | 95 | 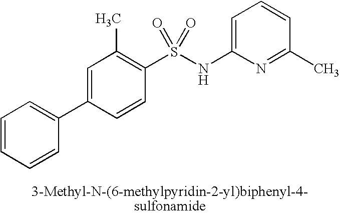 | N | NA | 339.1 |
| 167 | 36 | 100 | 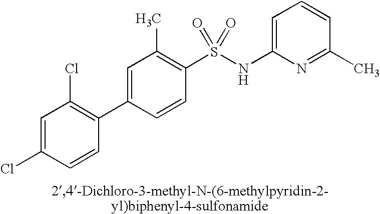 | N | NA | 406.9 |
| 168 | 2.8 | 96 | 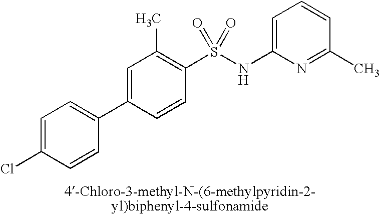 | N | NA | 373 |
| 169 | 19 | 93 | 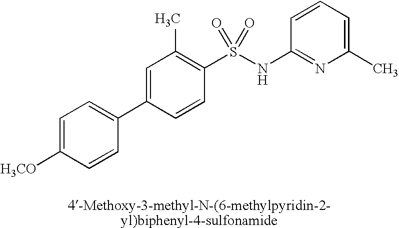 | N | NA | 369.1 |
| 170 | 8.1 | 93 | 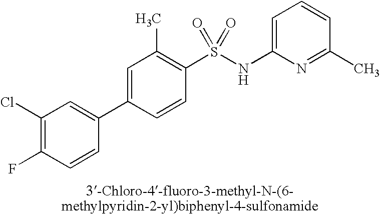 | N | NA | 391 |
| 171 | 6.9 | 93 | 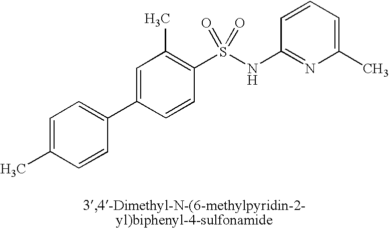 | N | NA | 353 |
| 172 | 48 | 82 | 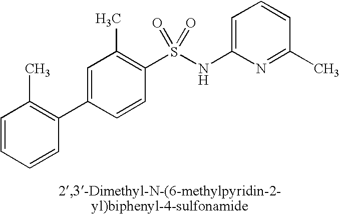 | N | NA | 353 |
| 173 | 9.6 | 97 | 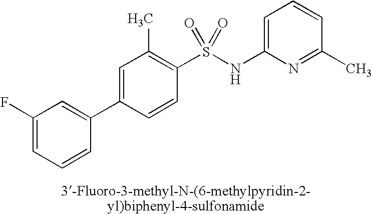 | N | NA | 356.9 |
| 174 | 3.2 | 98 | 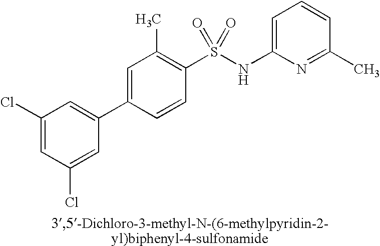 | N | NA | 406.9 |
| 175 | NA | 59 | 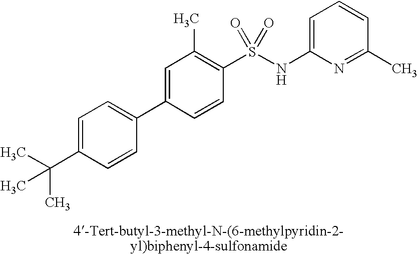 | N | NA | 395 |
| 176 | 5.2 | 100 | 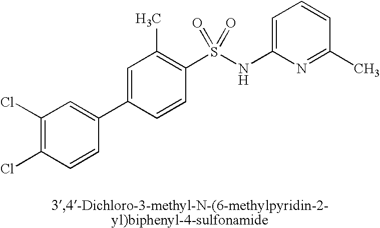 | N | NA | 406.9 |
| 177 | 20 | 85 | 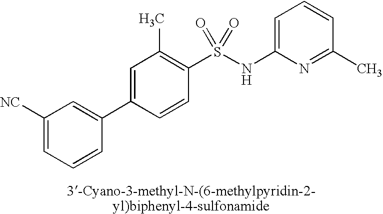 | N | NA | 364 |
| 178 | 8.2 | 95 | 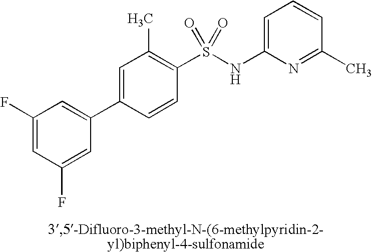 | N | NA | 374.9 |
| 179 | 6.2 | 95 | 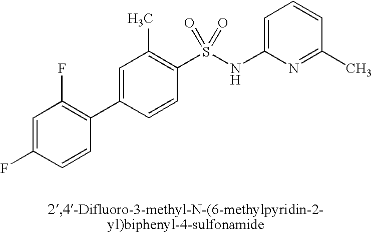 | N | NA | 374.9 |
| 180 | 75 | 65 | 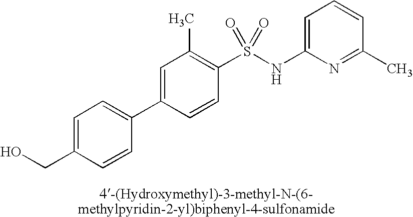 | N | NA | 369.1 |
| 181 | 53 | 83 | 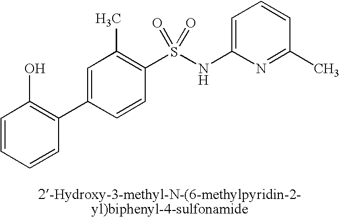 | N | NA | 355 |
| 182 | 23 | 79 | 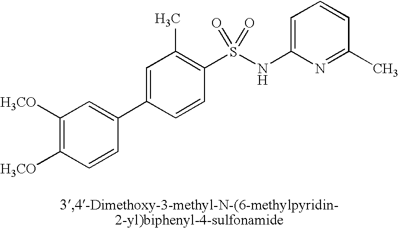 | N | NA | 399.1 |
| 183 | 14 | 91 | 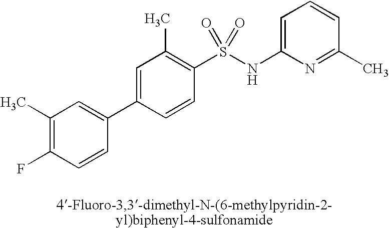 | N | NA | 371 |
| 184 | 12 | 97 | 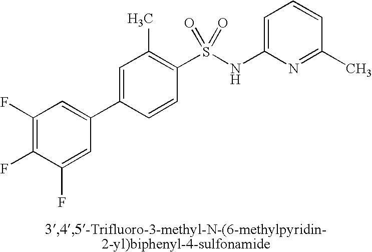 | N | NA | 392.9 |
| 185 | 46 | 71 | 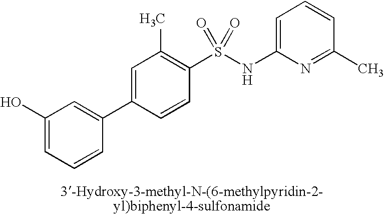 | N | NA | 355 |
| 186 | 9.3 | 95 | 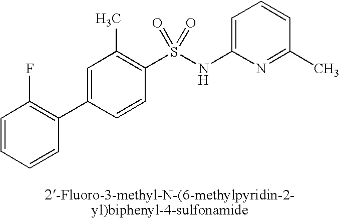 | N | NA | 356.9 |
| 187 | 9.5 | 93 | 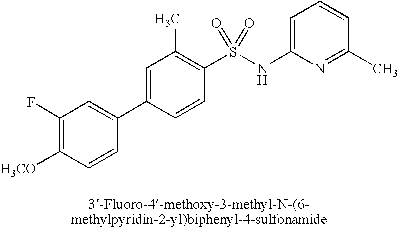 | N | NA | 386.9 |
| 188 | NA | 53 | 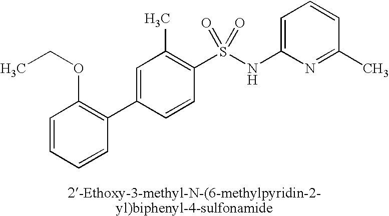 | N | NA | 383 |
| 189 | NA | 88 | 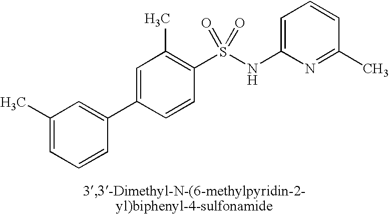 | N | NA | 353 |
| 190 | 35 | 78 | 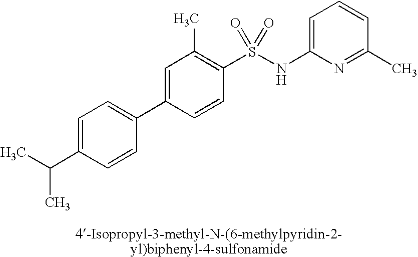 | N | NA | 381.1 |
| 191 | 11 | 100 | 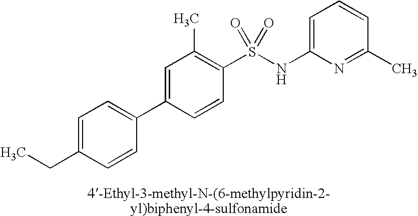 | N | NA | 367 |
| 192 | 15 | 81 | 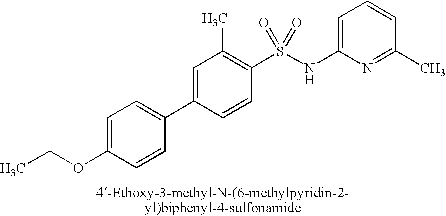 | N | NA | 383 |
| 193 | 39 | 78 | 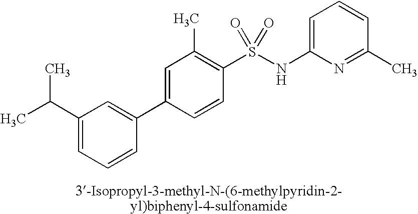 | N | NA | 381.1 |
| 194 | 47 | 74 | 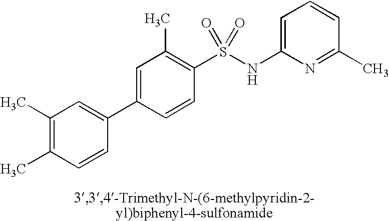 | N | NA | 387 |
| 195 | 17 | 82 | 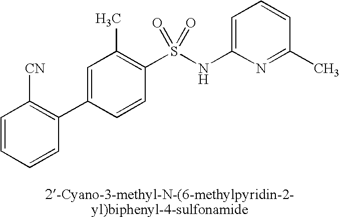 | N | NA | 364 |
| 196 | 7.1 | 90 | 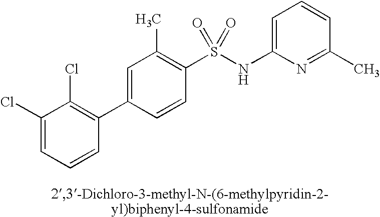 | N | NA | 406.9 |
| 197 | 29 | 89 | 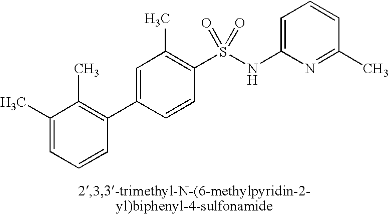 | N | NA | 367 |
| 198 | 7.5 | 100 | 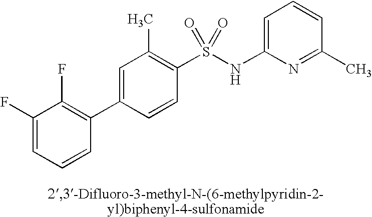 | N | NA | 374.9 |
| 199 | NA | 55 | 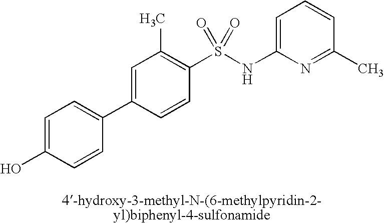 | N | NA | 355 |
| 200 | 21 | 88 | 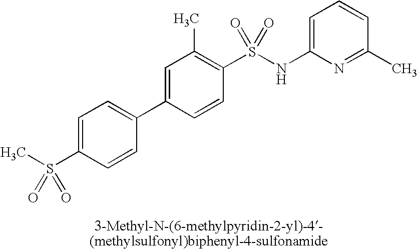 | N | NA | 416.9 |
| 201 | 43 | 75 | 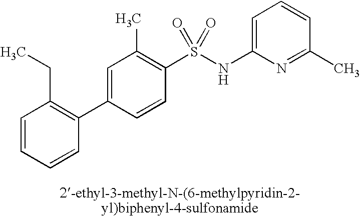 | N | NA | 367 |
| 202 | 14 | 88 | 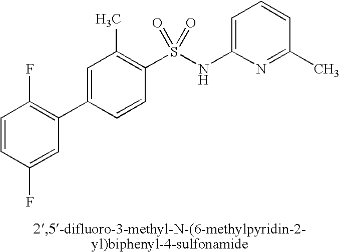 | N | NA | 374.9 |
| 203 | 19 | 86 | 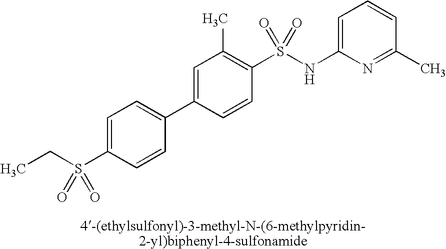 | N | NA | 431 |
| 204 | 10 | 85 | 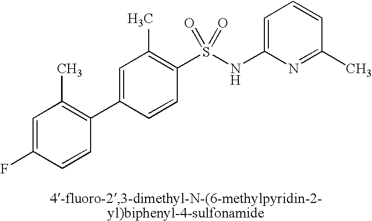 | N | NA | 371 |
| 205 | 19 | 86 | 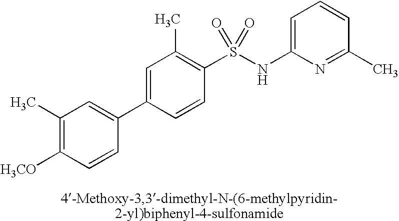 | N | NA | 383 |
| 206 | 20 | 87 | 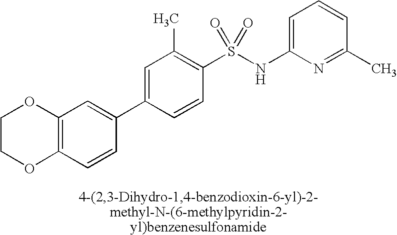 | N | NA | 397 |
| 207 | NA | 63 | 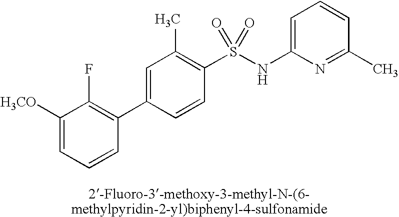 | N | NA | 396.9 |
| 208 | 6.7 | 95 | 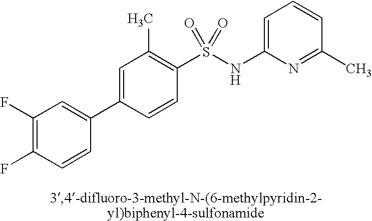 | N | NA | 374.9 |
| 209 | 18 | 88 | 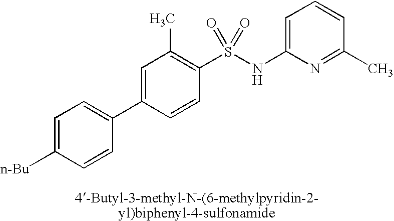 | N | NA | 395 |
| 210 | 47 | 74 | 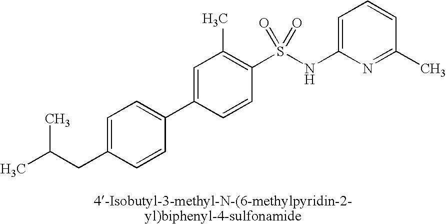 | N | NA | 395 |
| 211 | 23 | 81 | 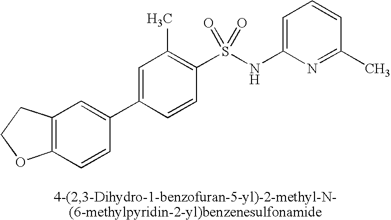 | N | NA | 380.9 |
| 212 | 47 | 75 | 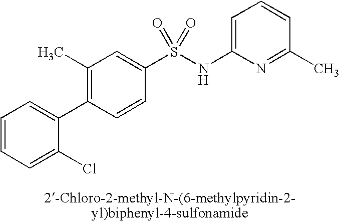 | N | NA | 373 |
| 213 | 31 | 77 | 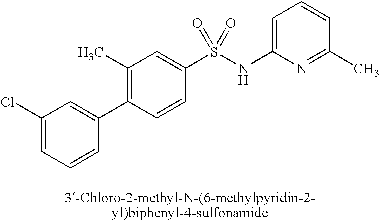 | N | NA | 373 |
| 214 | 36 | 72 | 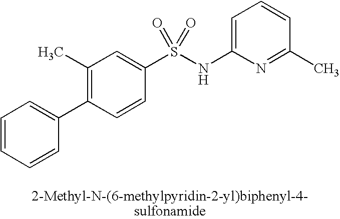 | N | NA | 338.9 |
| 215 | 16 | 90 | 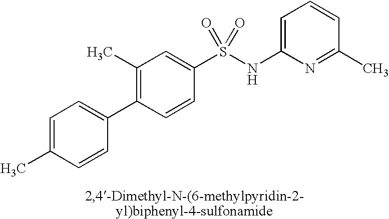 | N | NA | 353 |
| 216 | 43 | 73 | 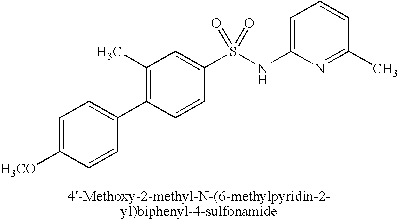 | N | NA | 368.9 |
| 217 | 5 | 95 | 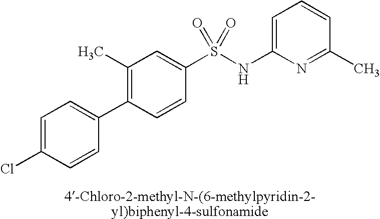 | N | NA | 373 |
| 218 | NA | 56 | 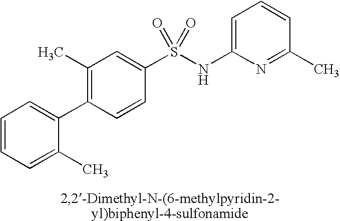 | N | NA | 353 |
| 219 | 17 | 68 | 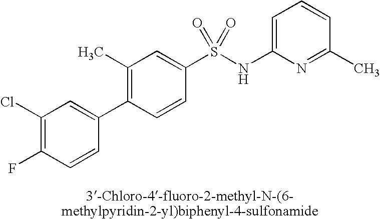 | N | NA | 391 |
| 220 | NA | 67 | 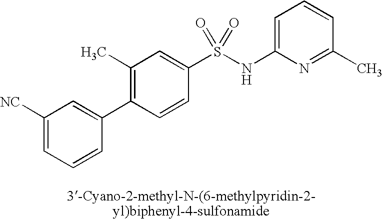 | N | NA | 364 |
| 221 | 18 | 87 | 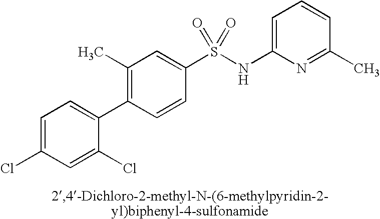 | N | NA | 406.9 |
| 222 | 10 | 90 | 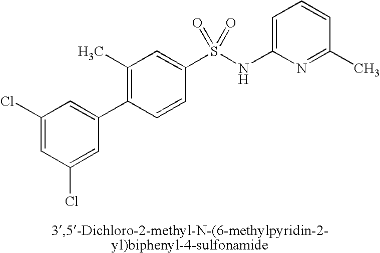 | N | NA | 406.9 |
| 223 | 320 | 79 | 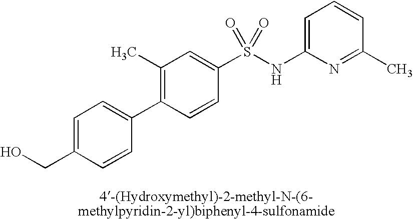 | N | NA | 368.9 |
| 224 | 19 | 93 | 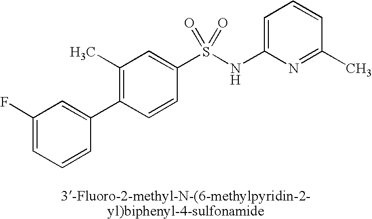 | N | NA | 356.9 |
| 225 | 16 | 28 | 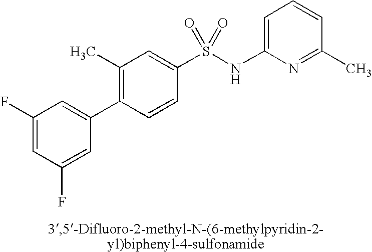 | N | NA | 374.9 |
| 226 | 460 | 94 | 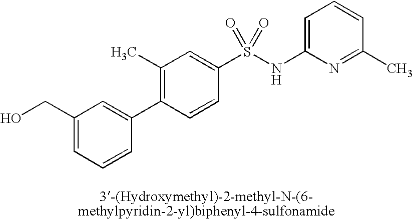 | N | NA | 369.1 |
| 227 | NA | 40 | 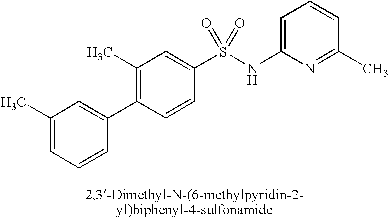 | N | NA | 353 |
| 228 | 29 | 74 | 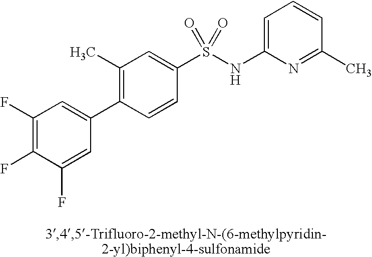 | N | NA | 392.9 |
| 229 | NA | 9 | 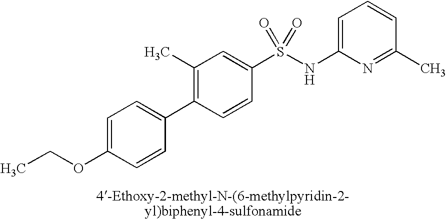 | N | NA | 383 |
| 230 | NA | 23 | 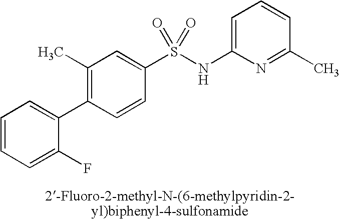 | N | NA | 356.9 |
| 231 | NA | 63 | 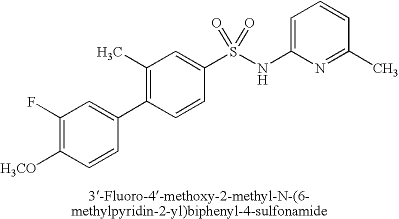 | N | NA | 386.9 |
| 232 | 660 | 78 | 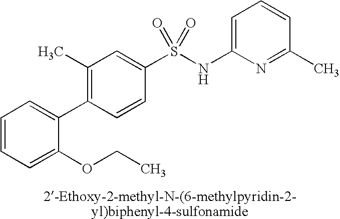 | N | NA | 383 |
| 233 | 110 | 78 | 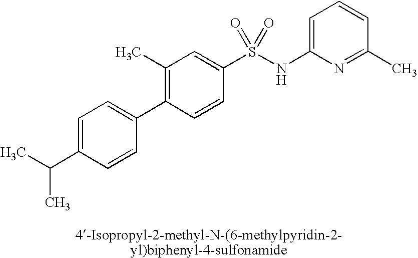 | N | NA | 381.1 |
| 234 | NA | 57 | 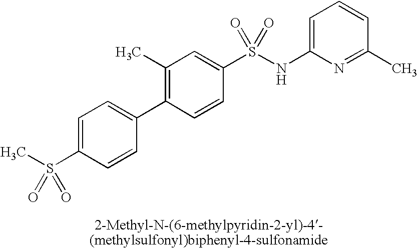 | N | NA | 416.9 |
| 235 | NA | 87 | 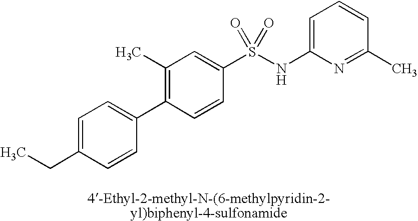 | N | NA | 367 |
| 236 | 110 | 81 | 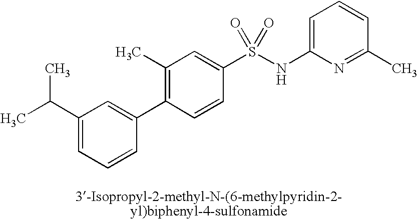 | N | NA | 381.1 |
| 237 | NA | 58 | 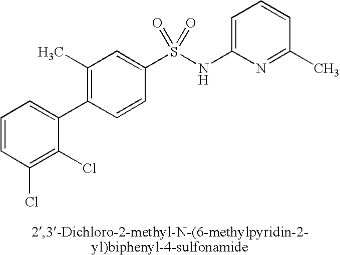 | N | NA | 406.9 |
| 238 | 27 | 82 | 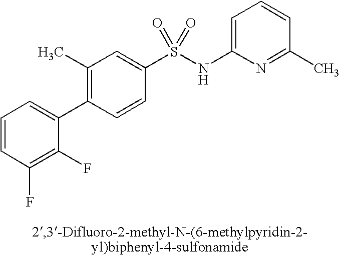 | N | NA | 374.9 |
| 239 | NA | 65 | 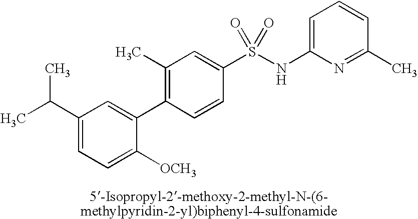 | N | NA | 411.1 |
| 240 | NA | 89 | 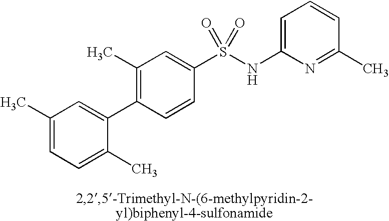 | N | NA | 367 |
| 241 | NA | 64 | 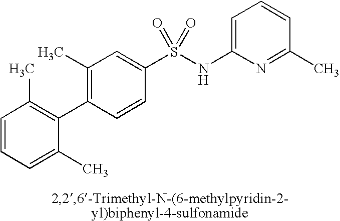 | N | NA | 367 |
| 242 | NA | 61 | 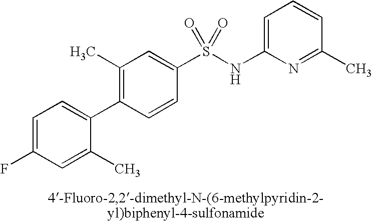 | N | NA | 371 |
| 243 | NA | 70 | 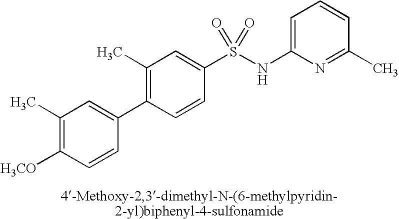 | N | NA | 383 |
| 244 | NA | 86 | 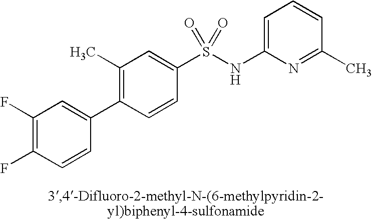 | N | NA | 374.9 |
| 245 | NA | 62 | 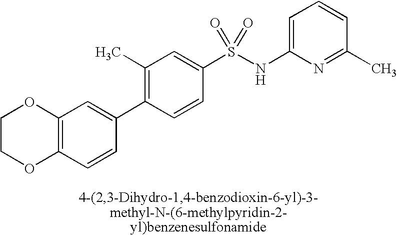 | N | NA | 397 |
| 246 | NA | 65 | 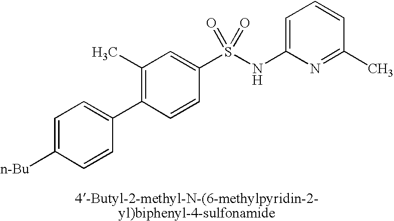 | N | NA | 395 |
| 247 | NA | 65 | 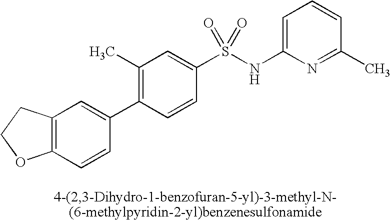 | N | NA | 360.9 |
| 248 | NA | 57 | 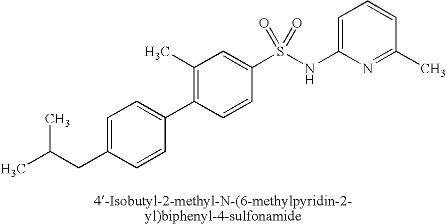 | N | NA | 395 |
| 249 | 5.8 | 96 | 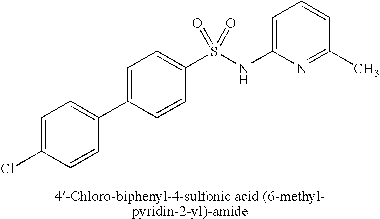 | O | (400 MHz, CD₃OD) δ: 8.01 (m, 2 H), 7.64 (m, 2 H), 7.55 (s, 1 H), 7.51 (m, 1 H), 7.44 (m, 1 H), 7.37 (m, 2 H), 7.00 (d, J=8.59 Hz, 1 H), 6.61 (d, J=7.33 Hz, 1 H), 2.42 (s, 3 H) | 359.1 |
| 250 | NA | 17 | 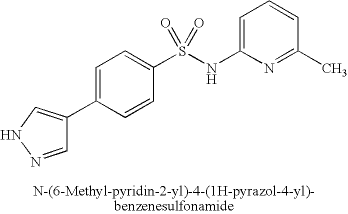 | O | (400 MHz, CDCl₃) δ: 7.97 (s, 2 H), 7.60 (d, J=8.3 Hz, 2 H), 7.62 (d, J=8.3 Hz, 2 H), 7.53 (dd, J=8.6, 7.6 Hz, 1 H), 7.03 (d, J=8.8 Hz, 1 H), 6.63 (d, J=7.3 Hz, 1 H), 2.30 (s, 3 H) | 315.1 |
| 251 | 29 | 83.6 | 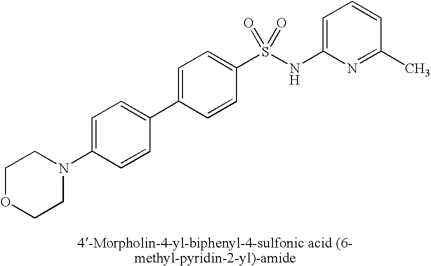 | O | (400 MHz, CDCl₃) δ: 2.54 (s, 3 H), 3.18 (m, 4 H), 3.82 (m, 4 H), 6.92 (m, 3 H), 7.46 (d, J=8.8 Hz, 2 H), 7.61 (m, 3 H), 7.84 (m, 1 H), 7.94 (d, J=8.6 Hz, 2 H) | 410.1 |
| 252 | NA | 44 | 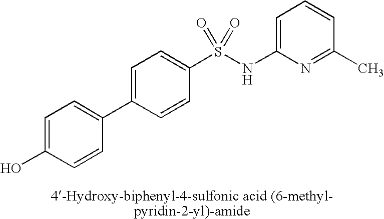 | O | (400 MHz, CDCl₃) δ: 2.34 (s, 3 H), 6.58 (d, J=7.3 Hz, 1 H), 6.78 (m, 2 H), 7.00 (d, J=8.3 Hz, 1 H), 7.35 (m, 2H), 7.45 (m, 1H), 7.49 (m, 2H), 7.85 (m, 2H) | 341.1 |
| 253 | 33.4 | 79.3 | 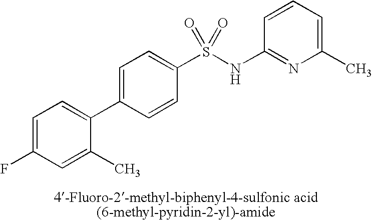 | O | (400 MHz, CDCl₃) δ 1.42 (t, J=7.0 Hz, 3 H), 6.61 (d, J=7.3 Hz, 1 H), 6.76 (dd, J=8.3, 2.5 Hz, 1 H), 6.79 (d, J=2.5 Hz, 1 H), 7.07 (d, J=8.3 Hz, 1 H), 7.19 (d, J=8.6 Hz, 1 H), 7.36 (d, J=8.3 Hz, 2 H), 7.54 (dd, J=8.6, 7.3 Hz, 1 H), 7.96 (d, J=8.3 Hz, 2 H) | 357.1 |
| 254 | 22.4 | 85.4 | 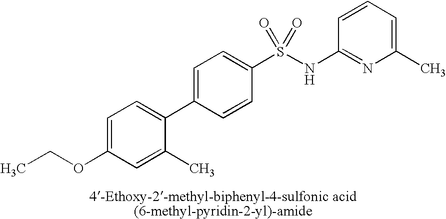 | O | (400 MHZ, CDCl₃) δ: 6.89 (d, J=1.8 Hz, 1 H), 7.38 (d, J=9.1 Hz, 1 H), 7.50-7.59 (m, 4 H), 7.63-7.68 (m, 1 H), 7.72 (d, J=8.8 Hz, 1 H), 7.74 (d, J=2.3 Hz, 1 H), 7.86 (d, J=8.3 Hz, 2 H), 9.34 (s, 1 H) | 383.1 |
| 255 | NA | 64.6 | 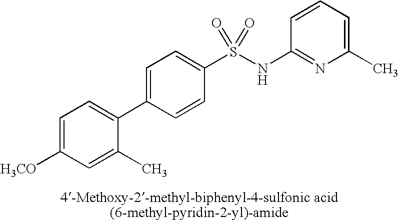 | O | (400 MHz, CDCl₃) δ: 1.42 (t, J=7.0 Hz, 3 H), 4.05 (q, 3 H), 6.61 (d, J=7.3 Hz, 1 H), 6.76 (dd, J=8.3, 2.5 Hz, 1 H), 6.79 (d, J =2.5 Hz, 1 H), 7.07 (d, J =8.3 Hz, 1 H), 7.19 (d, J =8.6 Hz, 1 H), 7.36 (d, J =8.3 Hz, 2 H), 7.54 (dd, J =8.6, 7.3 Hz, 1 H), 7.96 (d, J=8.3 Hz, 2 H) | 369.1 |
| 256 | 34.9 | 89.3 | 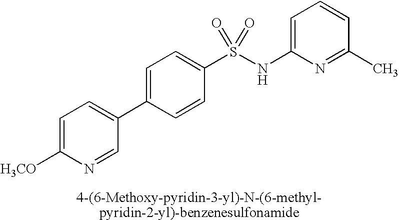 | O | (400 MHz, CDCl₃) δ: 8.38 (d, J=2.5 Hz, 1 H), 8.00 (d, J=8.1 Hz, 2 H), 7.77 (dd, J=8.6, 2.5 Hz, 1 H), 7.60 (d, J=8.1 Hz, 2 H) 7.46-7.54 (m, 1 H), 7.00 (d, J=8.6 Hz, 2 H), 6.83 (d, J=8.6 Hz, 2 H), 6.60 (d, J=7.3 Hz, 2 H) 3.98 (s, 3 H), 2.42 (s, 3 H) | 356.1 |
| 257 | 120 | 74.7 | 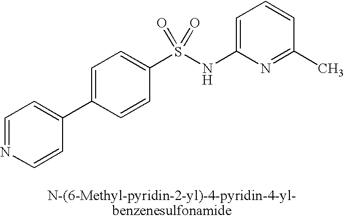 | O | (400 MHz, CDCl₃) δ: 8.83 (s, 2 H), 8.14 (d, J=8.3 Hz, 2 H), 7.69-7.77 (m, 3 H), 7.29-7.33 (m, 1 H), 6.78 (d, J=7.1 Hz, 1 H), 2.45 (m, 3 H) | NA |
| 258 | 5.9 | 100 | 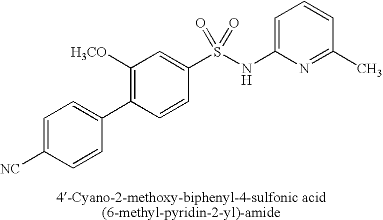 | O | (400 MHz, DMSO-d₆) δ: 7.89 (d, J=8.3 Hz, 2 H) 7.70 (d, J=8.3 Hz, 2 H) 7.66 (d, J=8.1 Hz, 1 H) 7.45-7.56 (m, 3 H) 7.13 (s, 1 H) 3.84 (s, 3 H) 2.33 (s, 3 H) | NA |
| 259 | NA | 30 | 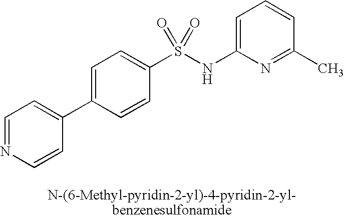 | P | (400 MHz, CDCl₃) δ 8.78 (d, J=4.80 Hz, 1 H), 8.10 (s, 2 H), 7.90 (m, 2 H), 7.81 (d, J=7.83 Hz, 1 H), 7.59 (d, J=8.84 Hz, 1 H), 7.42 (m, 1 H), 6.95 (d, J=7.58 Hz, 1 H), 2.59 (s, 3 H) | 326.1 |
| 260 | NA | 14.8 | 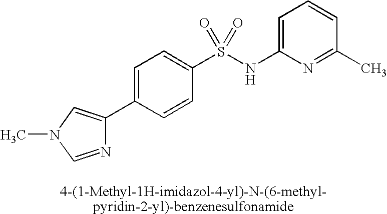 | P | (400 MHz, DMSO-d₆) δ: 9.37 (s, 1 H), 8.45 (s, 1 H), 8.15 (t, J=8.7 Hz, 4 H), 7.85 (d, J =7.8 Hz, 1 H) 7.28 (s, 1 H), 6.88 (s, 1 H), 4.09 (s, 3 H), 2.52 (s, 3 H) | 329.1 |
| 261 | NA | 2.6 | 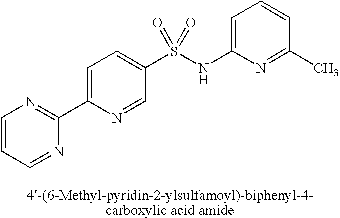 | P | (400 MHz, CDCl₃) δ: 8.97 (s, 1 H), 8.16-8.22 (m, 2 H), 8.11-8.16 (m, 2 H), 8.04 (d, J=2.3 Hz, 1 H), 7.93 (d, J=8.6 Hz, 1 H), 7.86-7.91 (m, 1 H), 7.68 (d, J8.8 Hz, 1 H), 6.99 (d, J=7.6 Hz, 1 H), 2.62 (s, 3 H) | 327.1 |
| 262 | NA | 45.7 | 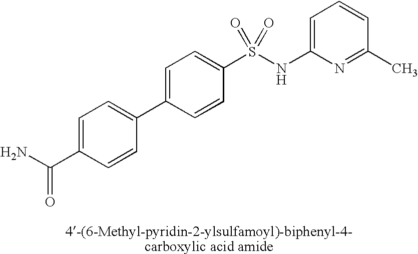 | Q | (400 MHz, CDCl₃) δ: 2.34 (s, 3 H), 6.58 (d, J=7.3 Hz, 1 H), 6.78 (m, 2 H), 7.00 (d, J=8.3 Hz, 1 H), 7.35 (m, 2H), 7.45 (m, 1H), 7.49 (m, 2H), 7.85 (m, 2H) | 368.1 |
| 263 | NA | 11.8 | 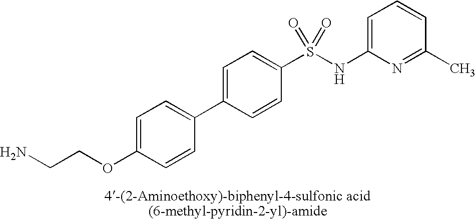 | R | (400 MHz, CDCl₃) δ: 2.30 (s, 3 H), 3.04 (m, 2 H), 4.05 (t, J=5.8 Hz, 1 H), 6.79 (m, 1 H), 7.08 (d, J =7.6 Hz, 1 H),7.40 (d, J =8.1 Hz, 2 H), 7.44 (m, 2 H), 7.55 (d, J=8.8 Hz, 2H), 7.64 (m, 3H) | 383.1 |
| 264 | NA | 39.7 | 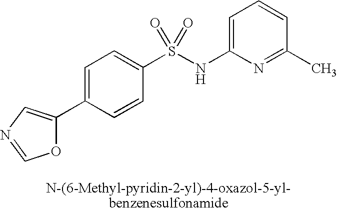 | S | (400 MHz, CDCl₃) δ: 8.21 (s, 1 H), 7.90 (d, J=8.3 Hz, 1 H), 7.62 (d, J=8.3 Hz, 1 H), 7.56 (s, 1 H), 7.54 (m, 1 H), 7.04 (m, 1 H), 6.56 (m, 1 H), 2.30 (s, 3 H) | 316.1 |
| 265 | NA | 11.4 | 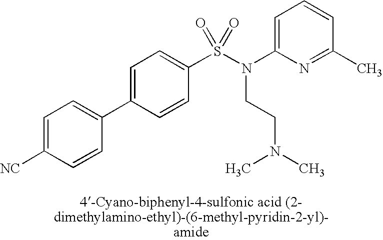 | T | (400 MHz, DMSO-d₆) δ: 10.10 (br s, J=3.8 Hz, 1 H), 7.90-6.07 (m, 6 H), 7.71-7.86 (m, 3 H), 7.41 (d, J=7.6 Hz, 1 H), 7.19 (d, J=7.6 Hz, 1 H), 4.17 (t, J=6.6 Hz, 2 H), 3.24 (m, 2 H), 2.82 (s, 6 H), 2.33 (s, 3 H) | 421.1693 |
| 266 | NA | 9.6 | 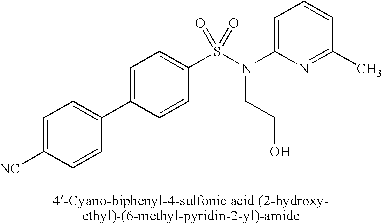 | U | (400 MHz, DMSO-d₆) δ: 7.91-8.01 (m, 6 H), 7.79 (d, J=8.3 Hz, 2 H), 7.73 (t, J=7.8 Hz, 1 H), 7.27 (d, J=7.8 Hz, 1 H), 7.11 (d, J=7.6 Hz, 1 H), 3.83 (t, J=6.3 Hz, 2 H), 3.47 (t, J=6.3 Hz, 2 H), 2.31 (s, 3 H) | 394.1218 |
| 267 | 22.6 | 81.8 | 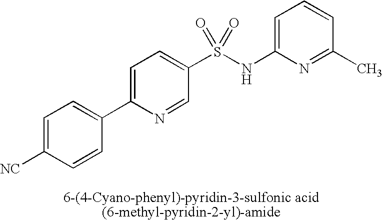 | V | (400 MHz, DMSO-d₆) δ: 2.16 (s, 3 H), 6.51 (s, 1 H), 7.01 (s, 1 H), 7.54 (s, 1 H), 7.83 (d, J=8.3 Hz, 2 H), 8.03-8.10 (m, 1 H), 8.12 (s, 2 H), 8.94 (s, 1 H) | 351.0 |
| 268 | 40 | 70 | 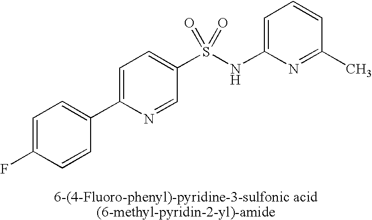 | V | (400 MHz, CDCl₃) δ: 9.18 (1H, s); 8.28 (1H, d); 8.01 (1H, dd), 7.77 (1H, d); 7.56 (1H, t); 7.18 (1H, d); 7.16 (1H, d); 7.10 (1H, d); 7.04 (1H, d); 6.58 (1H, d); 2.47 (1H, s); N-H not observed | 342 |
| 269 | NA | 2.4 | 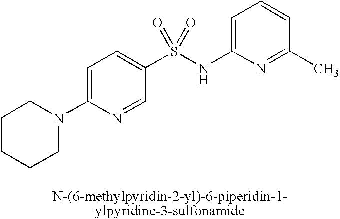 | W | (400 MHz, CDCl₃) δ: 8.65 (d, J=2.3 Hz, 1 H) 7.88 (dd, J=9.1, 2.5 Hz, 1 H) 7.26-7.46 (m, 1 H) 7.00 (d, J=8.6 Hz, 1 H) 6.40-6.61 (m, 2 H) 3.58 (d, J=5.1 Hz, 3 H) 2.93-3.14 (m, 3 H) 2.29 (s, 3 H) 1.47-1.81 (m, 6 H) | 333.1 |
| 270 | 10.7 | 94.8 | 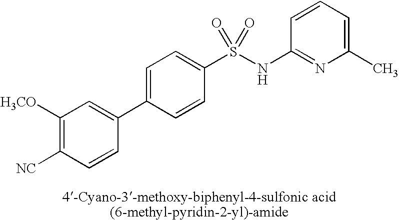 | X | (400 MHz, CDCl₃) δ: 2.46 (s, 3 H) 4.10 (s, 3 H) 6.60 (d, J=7.33 Hz, 1 H) 7.04 (d, J=8.84 Hz, 1 H) 7.10 (s, 1 H) 7.19 (dd, J=7.96, 1.14 Hz, 1 H) 7.44-7.50 (m, 1 H) 7.54 (d, J=7.58 Hz, 1 H) 7.62-7.67 (m, 2 H) 8.05 (d, J=8.34 Hz, 2 H) | 380.1 |
| 271 | 20 | 87.7 | 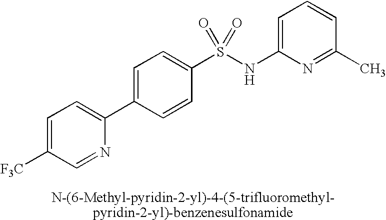 | X | (400 MHz, CDCl₃) δ: 8.97 (s, 1H), 8.19(d, J=8.3 Hz, 2 H), 8.13 (d, J=8.3 Hz, 1 H), 8.06 (dd, J=8.4, 2.15 Hz, 1 H) 7.93 (d, J =8.6 Hz, 1 H) 7.89 (m, 1 H) 7.68 (d, J=8.8 Hz, 1 H) 8.99 (d, J=7.58 Hz, 1 H) 2.62 (s, 3 H) | 393.0 |
| 272 | 12 | 100 | 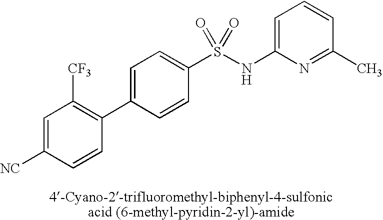 | X | (400 MHz, CDCl₃) δ: 7.98 (s, 1 H) 797-8.02 (m, 1 H) 7.95 (d, J=8.3 Hz, 2 H) 7.95 (d, J=8.3 Hz, 2 H) 7.80 (dd, J=8.0, 1.4 Hz, 1 H) 7.56-7.66 (m, 1 H) 7.44-7.52 (m, 1 H) 7.35-7.44 (m, 2 H) 7.32 (d, J=8.3 Hz, 2 H) 6.96 (d, J=8.8 Hz, 1 H) 6.52 (d, J=7.3 Hz, 1 H) 2.37-2.49 (m, 3 H) | 418.1 |
| 273 | 5.3 | 95.5 | 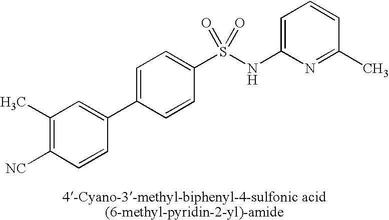 | X | (400 MHz, CDCl₃) δ: 7.95 (d, J=7.1 Hz, 2 H), 7.82-7.91 (m, 4 H), 7.72 (d, J=8.1 Hz, 1 H), 7.31 (t, J=8.0 Hz, 1 H), 6.21 (d, J=8.1 Hz, 1 H), 5.92 (d, J=7.6 Hz, 1 H), 2.56 (s, 3 H) | 364.1 |
| 274 | 3.5 | 94.4 | 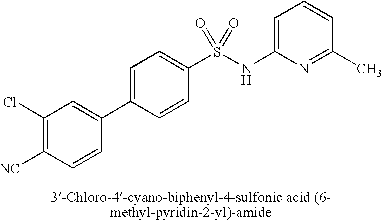 | X | (400 MHz, CDCl₃) δ: 8.11 (d, J=8.3 Hz, 2 H) 7.86 (t, J=8.1 Hz, 1 H) 7.77 (d, J=8.3 Hz, 1 H) 7.71 (s, 1 H) 7.70 (d, 1 H) 7.56 (d, J=8.6 Hz, 2 H) 6.92 (d, J=7.8 Hz, 1 H) 2.58 (s, 3 H) | 384.0 |
| 275 | NA | 85.7 | 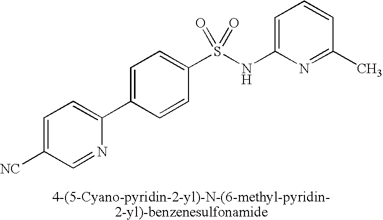 | X | (400 MHz, CDCl₃) δ: 10.19 (bs, 1 H) 8.94-8.96 (m, 1 H) 8.11-8.15 (m, 2 H) 8.02-8.09 (m, 3 H) 7.85-7.88 (m, 1 H) 7.50 (dd, J=8.6, 7.3 Hz, 1 H) 6.97 (d, J=8.0 Hz, 1 H) 6.57 (d, J=7.3 Hz, 1 H) 2.42 (s, 3 H) | 351.0910 |
| 276 | NA | 60.1 | 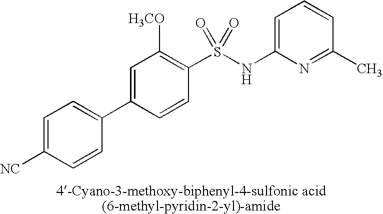 | Y | (400 MHz, CDCl₃) δ: 8.08 (d, J=8.1 Hz, 1 H), 7.74 (d, J=8.6 Hz, 2 H), 7.60-7.68 (m, 2 H), 7.43-7.51 (m, 1 H), 7.22 (dd, J=8.1, 1.5 Hz, 1 H), 7.09 (s, 1 H), 6.98 (d, J=8.8 Hz, 1 H), 6.65 (d, J=7.3 Hz, 1 H), 3.91 (s, 3 H), 2.43 (s, 2 H) | 380.1 |
| 277 | <1 | 100 | 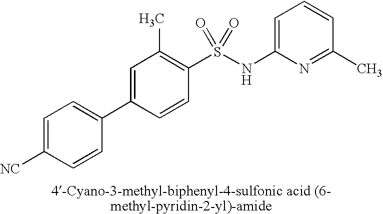 | Z | (400 MHz, DMS0-d₆) δ: 8.05 (1H, d), 7.96 (2H, d); 7.93 (2H, d); 7.89-7.68 (2H, m), 7.62 (1H, t); 7.01 (1H, bs), 6.61 (1H, bs); 2.69 (3H, s); 2.31 (3H, s), N-H proton not observed | 364 |
| 278 | NA | 42.8 | 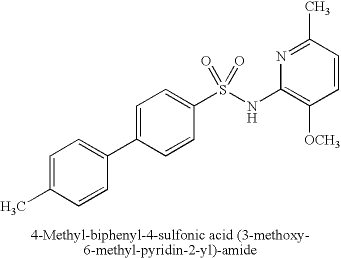 | Z | (400 MHz, CDCl₃) δ: 8.22 (s, 2H), 7.68-7.66 (m, 2H), 7.50-7.48 (m, 2H), 7.28-7.26 (2H, m), 6.90-6.88 (1H, m), 6.65 (s, 1H), 3.80 (s, 3H), 2.40 (s, 3H), 2.36 (s, 3H) | 369.1259 |
| 279 | 4.5 | 100 | 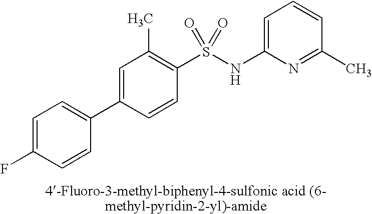 | Z | (400 MHz, DMSO-d₆) δ: 8.01 (1H, d); 7.77 (1H, d); 7.74 (1H, d); 7.65-7.56 (3H, m), 7.31 (1H, t); 6.98 (1H, bs); 6.62 (1H, bs); 2.67 (3H, s); 2.30 (3H, s), N-H proton not observed | 357 |
| 280 | 15 | 91 | 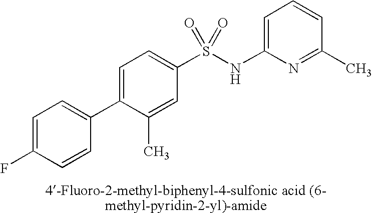 | Z | (400 MHz, DMSO-d₆) δ: 7.80 (1H, s); 7.73 (IH, d); 7.65 (1H, t); 7.42 (1H, d), 7.40 (1H, d); 7.34 (1H, d); 7.28 (1H, t); 7.09 (1H, bs); 6.68 (1H, bs); 2.32 (3H, s); 2.27 (3H, s), N-H proton not observed | 357 |
| 281 | 10 | 92.9 | 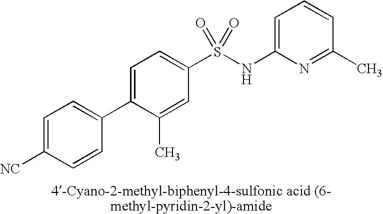 | Z | (400 MHz, DMSO-d₆) δ: 7.93 (2H, d); 7.82 (1H, s); 7.75 (1H, d); 7.65 (1H, t), 7.59 (2H, d); 7.38 (1H, d); 7.10 (1H, bs); 6.87 (1H, bs); 2.33 (3H, s); 2.28 (3H, s), N-H proton not observed | 364 |
| 282 | <1 | 100 | 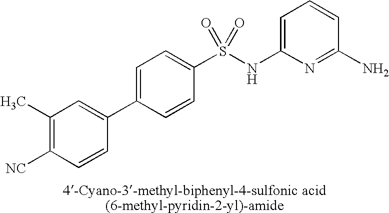 | AA | (400 MHz, DMSO-d₆) δ: 7.95 (d, J=7.1 Hz, 2 H) 7.82-7.91 (m, 4 H) 7.72 (d, J=8.1 Hz, 1 H) 7.31 (t, J=8.0 Hz, 1 H) 6.21 (d, J=8.1 Hz, 1 H) 5.92 (d, J=7.6 Hz, 1 H) 2.56 (s, 3 H) | 365.1 |
| 283 | 5.7 | 100 | 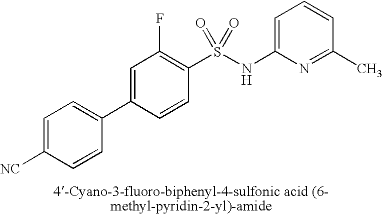 | AB | (400 MHz, CDCl₃) δ: 8.13 (t, J=7.7 Hz, 1 H) 7.77 (d, 2 H) 7.70-7.74 (m, 1 H) 7.66 (d, J=8.1 Hz, 2 H) 7.48 (d, J=8.6 Hz, 1 H) 7.35 (d, J=11.1 Hz, 1 H) 7.23 (s, 1 H) 6.78 (d, J=7.3 Hz, 1 H) 2.55 (s, 3 H) | 368.1 |
| 284 | 3.4 | 100 | 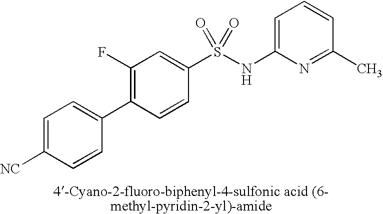 | AC | (400 MHz, CDCl₃) δ: 7.88 (dd, J=8.1, 1.5 Hz, 1 H) 7.73-7.84 (m, 4 H) 7.64 (d, J=7.3 Hz, 2 H) 7.57 (t, J=7.7 Hz, 1 H) 7.40 (d, J=8.8 Hz, 1 H) 6.84 (d, J=7.6 Hz, 1 H) 2.57 (s, 2 H) | 368.1 |
| 285 | 2.9 | 100 | 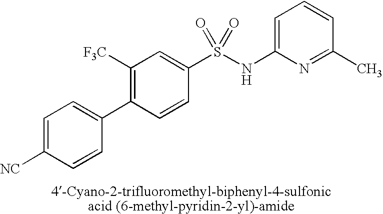 | AD | (400 MHz, CDCl₃) δ: 8.37 (d, J=1.5 Hz, 1 H) 8.23 (dd, J=8.1, 1.8 Hz, 1 H) 7.83 (dd, J=8.8, 7.6 Hz, 1 H) 7.73 (d, J=8.1 Hz, 2 H) 7.43-7.50 (m, 1 H) 7.39-7.44 (m, 2 H) 6.86 (d, J=7.3 Hz, 1 H) 2.59 (s, 3 H) | 418.1 |
| 286 | 6.2 | 100 | 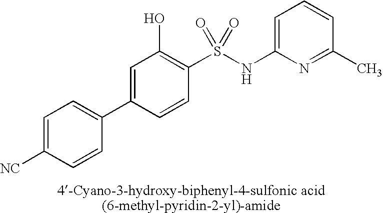 | AE | (400 MHz, DMSO-d₆) δ: 7.95 (d, J=8.6 Hz, 2 H), 7.80-7.90 (m, 3 H), 7.52-7.72 (m, 1 H), 7.30 (dd, J=8.2, 1.4 Hz, 1 H), 7.22 (d, J=1.8 Hz, 1 H), 6.72 (s, 1 H), 2.37 (m, 3 H) | NA |
| 287 | NA | 67.1 | 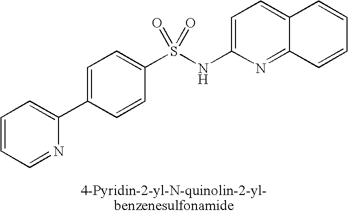 | AF | (400 MHz, Me0D) δ ppm 7.38-744 (m, 1 H) 7.57 (d, J=9.35 Hz, 2 H) 7.64-7.71 (m, 2 H) 7.78 (d, J=808 Hz, 1 H) 8.07-8.12 (m, 3 H) 8.13-8.24 (m, 4 H) 6.73 (d, J=4.29 Hz, 1 H) | 3626 |
| 288 | 11 | 91 | 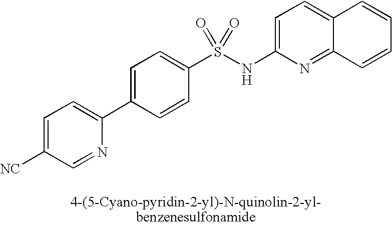 | AF | (400 MHz, CDCl₃) δ: 6.89 (d, J=9.35 Hz, 1 H) 7.38-7.46 (m, 2 H) 7.61-7.67 (m, 2 H) 7.88 (t, J=8.08 Hz, 2 H) 8.01-8.07 (m, 1 H) 8.11-8.17 (m, 4 H) 8.95 (s, 1 H) | 387.1 |
| 289 | 68 | 73.9 | 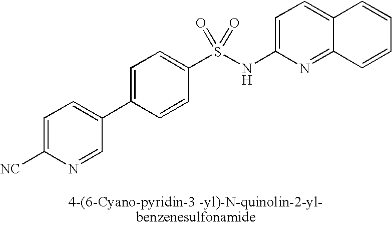 | AF | (400 MHz, CDCl₃) δ: 6.94-7.00 (m, 1 H) 7.39-7.49 (m, 2 H) 7.64-7.72 (m, 4 H) 7.79 (d, J=8.08 Hz, 1 H) 7.95 (d, J=9.35 Hz, 1 H) 8.00 (dd, J=8.08, 2.27 Hz, 1 H) 8.16 (d, J=8.59 Hz, 2 H) 8.92 (d, J=1.77 Hz, 1 H) | 387.1 |
| 290 | 12 | 100 | 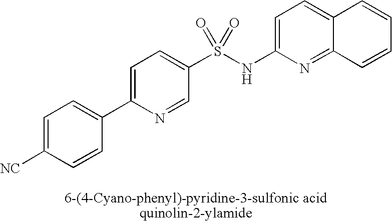 | AG | (400 MHz, DMSO-d₆) δ: 7.40 (t, J=7.58 Hz, 1 H) 7.57-7.64 (m, 2 H) 770 (t, J=7.33 Hz, 1 H) 7.86 (d, J=7.83 Hz, 1 H) 7.99 (d, J=8.59 Hz, 2 H) 8.24 (d, J=8.34 Hz, 1 H) 8.28-8.33 (m, 3 H) 8.37 (d, J=7.83 Hz, 1 H) 9.16 (s,1 H) | 387.1 |
| 291 | 27 | 92.5 | 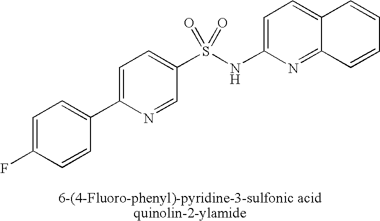 | AG | (400 MHz, DMSO-d₆) δ: 7.31-7.42 (m, 3 H) 7.56-7.62 (m, 2 H) 7.70 (t, J=7.33 Hz, 1 H) 7.85 (d, J=7.83 Hz, 1 H) 8.07-8.13 (m, 1 H) 8.14-8.20 (m, 2 H) 8.30 (d, J=9.35 Hz, 2 H) 9.10 (s, 1 H) | 380.1 |
| 292 | 49 | 72.4 | 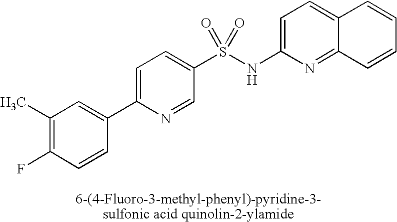 | AG | (400 MHz, DMSO-d₆) δ: 2.30 (d, J=1.26 Hz, 3 H) 7.26 (t, J=9.09 Hz, 1 H) 7.40 (t, J=7.45 Hz, 1 H) 7.59 H (d, J=8.08 Hz, 2 H) 7.69 (t, J=7.20 Hz, 1 H) 7.85 (d, J=7.58 Hz, 1 H) 7.97 (ddd, J=8.21, 5.31, 2.40 Hz, 1 H) 8.05-8.13 (m, 2 H) 8.29 (d, J=9.35 Hz, 2 H) 9.08 (s, 1 H) | NA |
| 293 | 6.4 | 100 | 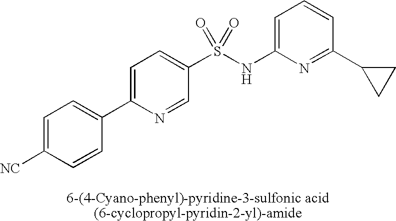 | AH | (400 MHz, CDCl₃), δ: 9.19 (d, J=1.5, 1 H), 8.35 (dd, J=10.8, 2.2 Hz, 1 H), 8.26 (d, J=7.8 Hz, 1 H), 8.07 (d, J=6.6 Hz, 1 H), 7.90 (d, J=8.6 Hz, 1 H), 7.56 (t, J=7.8 Hz, 1 H), 6.87 (d, ,J=8.4, 1 H), 6.82 (d, J =8.5, 1 H), 1.81-1.78 (m, H), 0.93-0.89 (m, 2 H), 0.73-0.70 | |
| # (m, 1 H) | 377.1072 | | | | | |
| 294 | 7.3 | 94.6 | 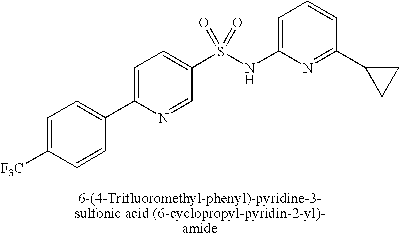 | AH | (400 MHz, CDCl₃), δ: 9.19 (d, J=1.5, 1 H), 8.35 (dd, J=10.8, 2.2 Hz, 1 H), 8.26 (d, J=7.8 Hz, 1 H), 8.07 (d, J=8.6 Hz, 1 H), 7.90 (d, J=8.6 Hz, 1 H), 7.56 (t, J=7.8 Hz, 1 H), 6.87 (d, J=8.4, 1 H), 6.82 (d, J=8.5, 1 H), 1.95-1.89 (m, 1 H), 0.93-0.89 | |
| # (m, 2 H), 0.73-0.70 (m, 1 H) | 420.0992 | | | | | |
| 295 | 2.3 | 100 | 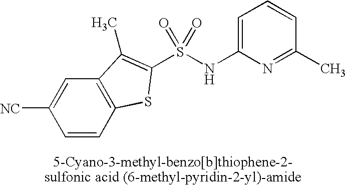 | AI | (400 MHz, DMSO-d₆), δ: 13.58 (br s, 1 H), 8.43 (s, 1 H), 8.21 (d, J=8.3 Hz, 1 H), 7.82 (dd, J=8.3, 1.3 Hz, 1 H), 7.72 (m, 1 H), 7.16 (m, 1 H), 8.68 (br d, J=7.3 Hz, 1 H), 2.63 (s, 3 H), 2.34 (s, 3 H) | 344.0522 |
| 296 | NA | 16.0 | 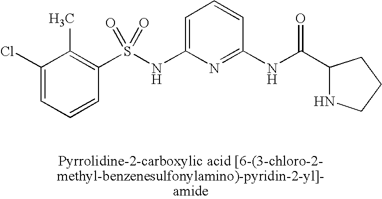 | AJ | (400 MHz, CDCl₃) δ: 7.98 (d, J=8.08 Hz, 1 H), 7.57 (s, 1H), 7.51 (m, 2H), 7.23 (t, J=8.0 Hz, 1H), 6.68 (d, J=8.34 Hz, 1 H), 3.52 (d, J=6.8 Hz, 1 H), 3.28 (m, 4 H), 2.29 (m, 1H), 2.12 (dd, J=10.99, 5.68 Hz, 1 H) | 394.0 |
| 297 | NA | 3.4 | 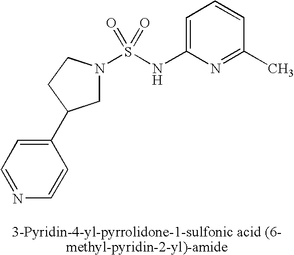 | AK | (400 MHz, CD₃CN) δ: 8.35 (br s, 1 H) 8.42 (dd, J =4.6, 1.8 Hz, 2 H), 7.53 (t, J=8.4 Hz, 1 H), 7.15 (d, J=4.8 Hz, 2 H), 6.84 (d, J=8.3 Hz, 1 H), 6.78 (d, J=7.5 Hz, 1 H); 3.90 (dd, J=6.5, 4.6 Hz, 1 H), 3.60-3.57 (m, 1 H), 3.45-3.27 (m, 4 H), 2.28-2.20 (m, 1 H) | 319.0 |
| 298 | NA | 13.3 | 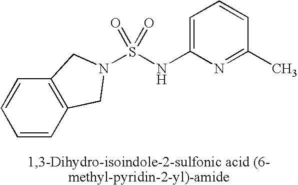 | AK | (400 MHz, CDCl₃) δ: 2.43 (3 H, s) 4.76 (4 H, s) 6.65 (1 H, d, J=7.3 Hz) 7.05 (1 H, d, J=8.6 Hz) 7.19-7.23 (2 H, m) 7.25-7.29 (2 H, m) 7.52 (1 H, dd, J=8.5, 7.4 Hz) | 288 |
| 299 | NA | 55.7 | 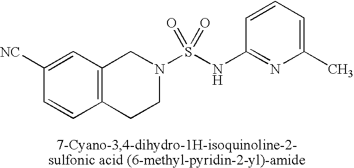 | AK | (400 MHz, CDCl₃) δ: 7.47 (dd, J=8.6, 7.3 Hz, 1 H), 7.42 (dd, J=7.8, 1.5 Hz, 1 H), 7.33 (s, 1 H), 7.21 (d, J=8.1 Hz, 1 H), 6.81 (d, J=8.6 Hz, 1 H), 6.53 (d, J=7.1 Hz, 1 H), 4.45 (s, 2 H), 3.56 (t, J=5.9 Hz, 2 H), 2.99 (t, J=5.8 Hz, 2 H), 2.39 (s, 3 H) | 304.2 |
| 300 | 11 | 88.3 | 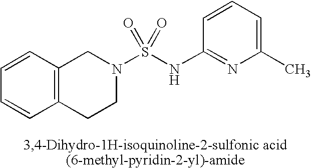 | AK | (400 MHz, CDCl₃) δ: 7.50 (dd, J=8.5, 7.4 Hz, 1 H), 7.13-7.16 (m, 2 H), 7.07-7.11 (m, 1 H), 7.01-7.05 (m, 1 H), 6.97 (s, 1 H), 6.62 (d, J=7.3 Hz, 1 H), 4.48 (s, 2 H), 3.57 (t, J =5.9 Hz, 2 H), 2.93 (t, J =5.9 Hz, 2 H), 2.41 (s, 3 H) | 329.2 |
| 301 | 4.9 | 100 | 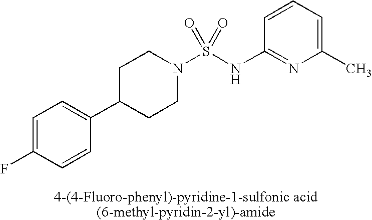 | AK | (400 MHz, CDCl₃) δ: 7.53 (dd, J=8.3, 7.6 Hz, 1 H), 7.07-7.13 (m, 2 H), 6.97 (td, J=8.8, 2.3 Hz, 3 H), 6.65 (d, J=7.3 Hz, 1 H), 3.91 (dd, J=10.2, 1.9 Hz, 2 H), 2.86 (td, J=12.2, 2.3 Hz, 2 H), 2.51-2.59 (m, 1 H), 1.87 (s, 1 H) 2.44 (s, 3 H), 1.84 (d, J=1.5 Hz, | |
| # 1 H), 1.72 (qd, J=12.7, 3.9 Hz, 2 H) | 350.1 | | | | | |
| 302 | NA | 18.1 | 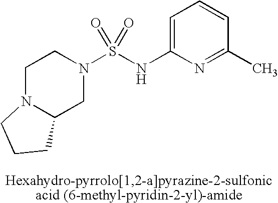 | AK | (400 MHz, CD₃CN) δ: 8.35 (br s, 1 H), 7.60 (t, J=8.4 Hz, 1 H), 7.02 (d, J=8.1 Hz, 1 H), 6.77 (d, J=7.6 Hz, 1 H), 3.77 (dd, J =10.4, 1.5 Hz, 1 H), 3.77 (dd, J=10.4, 1.5 Hz, 1 H), 3.61 (d, J=4.8 Hz, 4 H), | |
| # 3.04 (m, 3 H), 2.61 (t, J=10.4 Hz, 1 H), 2.41 (s, 3 H), 2.20-1.97 (m, 3 H), 1.86-1.71 (m, 3 H), 1.41-1.33 (m, 1 H) | 297.0 | | | | | |
| 303 | 7.6 | 89.3 | 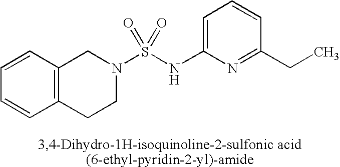 | AK | (400 MHz, CDCl₃) δ ppm 1.28 (t, J=7.58 Hz, 3 H), 2.70 (q, J=7.58 Hz, 2 H), 2.94 (t, J=5.94 Hz, 2 H), 3.57 (t, J=5.94 Hz, 2 H), 4.48 (s, 2 H), 6.63 (d, J =7.33 Hz, 1 H), 6.96-7.00 (m, 1 H), 7.03 (dd, J =5.18, 3.66 Hz, 1 H), 7.07-7.11 (m, 1 H), 7.12-7.15 (m, | |
| # 2 H), 7.55 (dd, J =8.46, 7.45 Hz, 1 H) | 318.1 | | | | | |
| 304 | 20 | 79.6 | 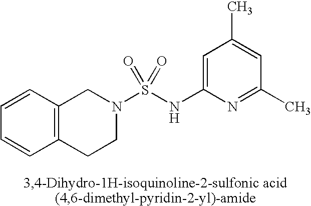 | AK | (400 MHz, CDCl₃) δ: 2.23 (s, 3 H) 2.36 (s, 3 H) 2.92 (t, J=5.81 Hz, 2 H) 3.52 (t, J=5.94 Hz, 2 H) 4.44 (s, 2 H) 6.34 (s, 1 H) 6.73 (s, 1 H) 7.00-7.05 (m, 1 H) 7.07-7.11 (m, 1 H) 7.11-7.16 (m, 2 H) | 318.2 |
| 305 | NA | 1.1 | 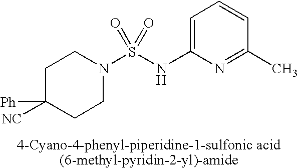 | AK | (400 MHz, CDCl₃) δ: 10.36 (br s, 1 H), 7.52 (t, J=8.0 Hz, 1H), 7.45-7.28 (m, 5H), 7.00 (d, J=8.0 Hz, 1H), 6.57 (d, J=8.0 Hz, 1H), 4.00-3.80 (m, 2 H), 3.30-3.15 (m, 2 H), 2.45 (s, 3 H), 2.20-2.05 (m, 4 H) | 357.1379 |
| 306 | 7.4 | 100 | 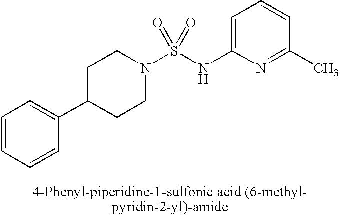 | AK | (400 MHz, CDCl₃) δ: 9.56 (br s, 1 H), 7.53 (t, J=8.0 Hz, 1H), 7.34-7.24 (m, 2H), 7.24-7.12 (m, 3 H), 7.05 (d, J=8.0 Hz, 1H), 6.67 (d, J=8.0 Hz, 1H), 3.97-3.87 (m, 2 H), 2.93-2.80 (m, 2 H), 2.62-2.50 (m, 1 H), 2.47 (s, 3 H), 1.93-1.83 (m, 2 H), 1.83-1.67 (m, 2 H) | 332.1432 |
| 307 | NA | 29.5 | 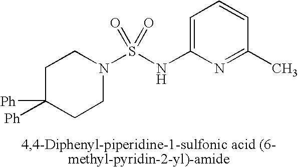 | AK | (400 MHz, CDCl₃) δ: 9.60 (br s, 1 H), 7.47 (t, J=8.0 Hz, 1 H), 7.30-7.10 (m, 10 H), 6.99 (d, J=8.0 Hz), 6.63 (d, J=8.0 Hz), 3.40-3.32 (m, 4 H), 2.48-2.41 (m, 4 H), 2.38 (s, 3 H) | 408.1739 |
| 308 | <1 | 95.7 | 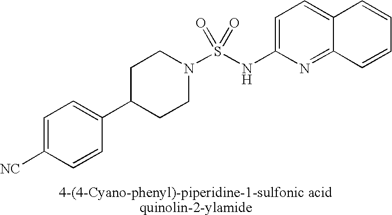 | AK | (400 MHz, CDCl₃) δ: 1.65-1.76 (m 4 H) 2.42-2.52 (m, 1 H) 2.64 (td, J=11.56, 3.66 Hz, 2 H) 3.75 (d, J =11.87 Hz, 2 H) 6.68 (d, J =9.35 Hz, 1 H) 7.11 (d, J =8.34 Hz, 2 H) 7.13-7.21 (m, 2 H) 7.37-7.46 (m, 4 H) 7.67 (d, J=9.60 Hz, 1 H) | 393.1 |
| 309 | 7.6 | 90 | 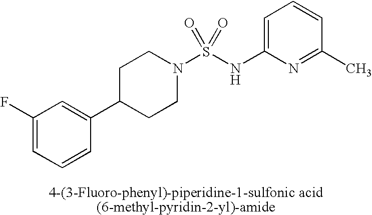 | AK | (400 MHz, CDCl₃) δ: 1.66-1.82 m, 2 H) 1.83-1.93 (m, 2 H) 2.45 (s, 3 H) 2.52-2.64 (m, 1 H) 2.83-2.98 (m, 2 H) 3.88-3.97 (m, 2 H) 6.67 (d, J=7.07 Hz, 1 H) 6.88-7.23 (overlapping m, 5 H), 7.50-7.60 (m, 1 H) | 350.1 |
| 310 | 18 | 100 | 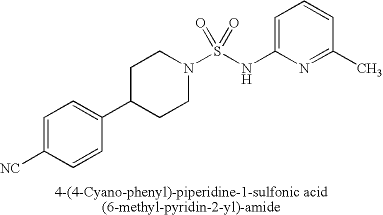 | AK | (400 MHz, CD₃CN) δ: 8.91 (br s, 1 H), 8.68 (d, J=8.3 Hz, 2 H), 7.61 (t, J=8.6, 1 H), 7.39 (d, J=8.1 Hz, 2 H), 7.01 (d, J=8.6 Hz, 1 H), 6.80 (d, J=7.3 Hz, 1 H), 3.87 (dd, J=10.1, 2.1 Hz, 2 H), 2.88 (td, J =12.3, 2.3 Hz, 2 H), 2.42 (s, 3 H), | |
| # 1.86 (bd, J=12.9 Hz, 2 H), 1.69 (qd, J=12.6, 4 Hz, 2 H) | 357.1 | | | | | |
| 311 | 2.6 | 100 | 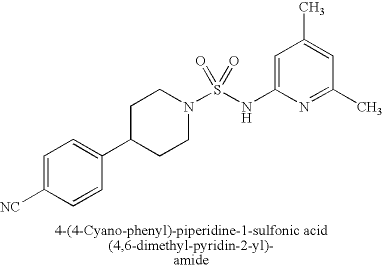 | AK | (400 MHz, DMSO-d₆) δ: 7.56 (d, J=8.4 Hz, 2 H), 7.25 (d, J=8.1 Hz, 2 H), 6.70 (bs, 1 H), 6.32 (bs, 1 H), 3.51 (d, J=7.6 Hz, 2 H), 2.64-2.42 (m, 3 H), 2.11 (s, 3 H), 2.03 (s, 3 H), 1.61 (d, J=11.4 Hz, 1 H), 1.49-1.34 (m, 2 H) | 371.1 |
| 312 | NA | 6.9 | 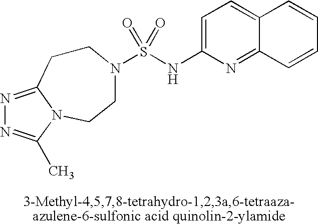 | AK | (400 MHz, CDCl₃) δ: 2.42 (s, 3 H) 334 (dd, J=6.06, 4.04 Hz, 2 H) 360 (dt, J=5.05, 2.53 Hz, 2 H) 3.72 (dd, J=5.94, 2.40 Hz, 2 H) 4.21 (d, J=7.07 Hz, 2 H) 6.93 (d, J=8.84 Hz, 1 H) 7.32-7.41 (m, 2 H) 7.57-7.65 (m, 2 H) 7.87 (d, J=9.60 Hz, 1 H) | 359.1 |
| 313 | NA | 27.2 | 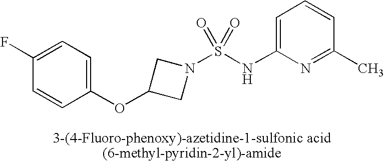 | AK | (400 MHz, DMSO-d₆) δ: 7.67 (m, 1 H), 7.04-7.17 (m, 3 H), 6.84 (dd, J = 9.1, 4.3 Hz, 2 H), 6.70 (br s, 1 H), 4.89 (m, 1 H), 4.15 (br s, 2 H), 3.82 (br s, 2 H), 2.33 (s, 3 H) | 338.0974 |
| 314 | 52 | 71.8 | 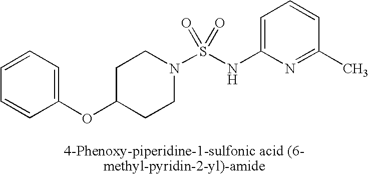 | AK | (400 MHz, CDCl₃) δ: 7.61 (dd, J=8.6, 7.6 Hz, 1 H), 7.26 (m, 2 H), 709 (d, J =8.6 Hz, 1 H), 6.94 (t, J =7.3 Hz, 1 H), 6.88 (d, J =7.8 Hz, 2 H), 6.71 (d, J =7.3 Hz, 1 H), 4.45 (m, 1 H), 3.52 (m, 2 H), 3.33 (m, 2 H), 2.48 (s, 3 H), 2.00 (m, 2 H), 1.90 (m, 2 H) | 348.1376 |
| 315 | 25 | 86.1 | 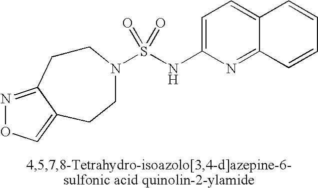 | AK | (400 MHz, DMSO-d₆) δ: 2.73-2.81 (m, 2 H) 3.01 (ddd, J=5.37, 2.59, 2.40 Hz, 2 H) 3.46 (s, 2 H) 3.52 (s, 2 H) 7.30-7.42 (m, 2 H) 7.57-7.67 (m, 2 H) 7.80 (d, J=8.08 Hz, 1 H) 8.20 (d, J=9.60 Hz, 1 H) 8.60 (s, 1 H) | 345.1 |
| 316 | 67 | 67.4 | 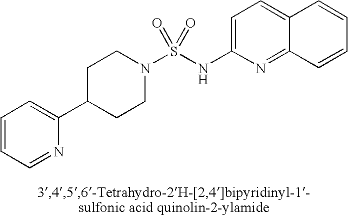 | AK | NA | 369.5 |
| 317 | <1 | 100 | 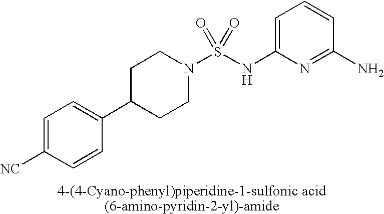 | AL | (400 MHz, CD₃OD) δ: 1.67 (qd, J=12.59, 3.92 Hz, 2 H) 1.78-1.85 (m, 2 H) 2.65-2.74 (m, J=12.16, 12.16, 3.60, 3.41 Hz, 1 H) 2.91 (td, J=12.44, 2.40 Hz, 2 H) 3.85-3.93 (m, 2 H) 6.13 (d, J=8.08 Hz, 1 H) 6.39 (dd, J=8.08, 0.51 Hz, 1 H) 7.35-7.40 (m, 3 H) 7.60-7.66 (m, 2 H) | 356.2 |

Various embodiments of the present invention have been described above but a person skilled in the art realizes further minor alterations that would fall into the scope of the present invention. The breadth and scope of the present invention should not be limited by any of the above-described exemplary embodiments, but should be defined only in accordance with the following claims and their equivalents.

1. A compound of formula (I): 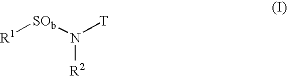
wherein:
R¹ is selected from the group consisting of (C₁-C₆)alkyl, —(CR³R⁴)ₜ(C₃-C₁₂)cycloalkyl, —(CR³R⁴)ₜ(C₆-C₁₂)aryl, and —(CR³R⁴)ₜ(4-10)-membered heterocyclyl wherein said —(CR³R⁴)ₜ(4-10)-membered heterocyclyl is optionally substituted on a nitrogen atom by a substituent selected from the group consisting of —CF₃, —CHF₂, —CH₂F, (C₁-C₆)alkyl, (C₂-C₆)alkenyl, (C₂-C₆)alkynyl, —(C═O)—R³, —(C═O)—O—R³, —(CR³R⁴)ₜ(C₆-C₁₂ aryl), —(CR³R⁴)ₜ(4-10)-membered heterocyclyl, —(CR³R⁴)ₜ—(C═O)(CR³R⁴)ₜ(C₆-C₁₂)aryl, —(CR³R⁴)ₜ—(C═O)(CR³R⁴)ᵤ(4-10)-membered heterocyclyl, —(CR³R⁴)ₜO(CR³R⁴)ᵤ(C₆-C₁₂)aryl, —(CR³R⁴)ₜO(CR₃R⁴)ᵤ(4-10)-membered heterocyclyl, —(CR³R⁴)ₜS(O)ⱼ(CR³R⁴)ₜ(C₆-C₁₂)aryl, and —(CR³R⁴)ₜS(O)ⱼ(CR³R⁴)ᵥ(4-10)-membered heterocyclyl;
b and k are each independently selected from 1 and 2;
j is selected from the group consisting of 0, 1, and 2;
t, u, p, q, and v are each independently selected from the group consisting of 0, 1, 2, 3, 4, and 5;
T is a (6-10)-membered heterocyclyl containing at least one nitrogen atom;
R² is selected from the group consisting of H, (C₁-C₆)alkyl, —(CR³R⁴)ₜ(C₃-C₁₂)cycloalkyl, —(CR³R⁴)ₜ(C₆-C₁₂)aryl, and —(CR³R⁴)ₜ(4-10)-membered heterocyclyl;
each R³ and R⁴ is independently selected from H and (C₁-C₆)alkyl;
the carbon atoms of T, R¹, R², R³ and R⁴ may each be optionally substituted by 1 to 5 R⁵ groups;
each R⁵ group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(C═O)—R⁶, —(C═O)—O—R⁶, —R⁶—O—R⁷, —R⁶—(C═O)—R⁷, —R⁶—(C═O)—OR⁷, —O—R⁶, —O—(C═O)—R⁷, —O—(C═O)—NR⁷R⁸, —NR⁸—(C═O)—R⁹, —R⁷—(C═O)—NR⁸R⁹, —(C═O)—NR⁸R⁹, —R⁶—(C═O)—NR⁸R⁹—NR⁸R⁹, —NR⁸OR⁹, —S(O)ₖNR⁸R⁹, —S(O)ⱼ(C₁-C₆)alkyl, —O—S(O)ₖ—R⁹, —NR⁸—S(O)ₖ—R⁹, —(CR⁸R⁹)ᵥ(C₃-C₁₂)cycloalkyl-(CR¹⁰R¹¹)ᵥ(C₆-C₁₂aryl), —(CR¹⁰R¹¹)ᵥ(4-10)-membered heterocyclyl, —(CR¹⁰OR¹¹)_{q}(C═O)(CR¹⁰OR¹¹)ᵥ(C₆-C₁₂)aryl, —(CR¹⁰R¹¹)_{q}—(C═O)(CR¹⁰R¹¹)ᵥ(4-10)-membered heterocyclyl, —(CR¹⁰R¹¹)ᵥO(CR¹⁰R¹¹)_{q}(C₆-C₁₂)aryl, —(CR¹⁰R¹¹)ᵥO(CR¹⁰R¹¹)_{q}(4-10)-membered heterocyclyl, —(CR¹⁰R¹¹)_{q}S(O)ⱼ(CR¹⁰OR¹¹)ᵥ(C₆-C₁₂)aryl, and —(CR¹⁰OR¹¹)_{q}S(O)ⱼ(CR¹⁰OR¹¹)ᵥ(4-10)-membered heterocyclyl;
any 1 or 2 carbon atoms of any (4-10)-membered heterocyclyl of the foregoing R⁵ groups are optionally substituted with an oxo (═O);
any nitrogen atom of any (4-10)-membered heterocyclyl of the foregoing R⁵ group is optionally substituted with (C₁-C₆)alkyl or —(CR¹⁰R¹¹)ᵥ(C₆-C₁₂)aryl;
any carbon atom of any (C₁-C₆)alkyl, any (C₆-C₁₂)aryl, and any (4-10)-membered heterocyclyl of the foregoing R⁵ groups are optionally substituted with 1 to 3 substituents independently selected from halo, hydroxy, cyano, nitro, —CF₃, —CFH₂, —CF₂H, trifluoromethoxy, azido, —OR¹², —R¹²—O—R¹³, —(C═O)—R¹², —(C═O)—O—R¹³, —O—(C═O)—R¹³, —NR¹³—(C═O)—R¹⁴, —(C═O)—NR¹⁵R¹⁶, —NR¹⁷R¹⁸, —SR¹⁴, —S(O)ⱼ(C₁-C₆)alkyl, —NR¹⁴OR¹⁵, (C₁-C₆)alkyl, (C₂-C₆)alkenyl, (C₂-C₆)alkynyl, —(CR¹⁶R¹⁷)ᵤ(C₆-C₁₂)aryl, and —(CR¹⁶R¹⁷)ᵤ(4-10)-membered heterocyclyl;
each R⁶, R⁷, R⁸, R⁹, R¹⁰, R¹¹, R¹², R¹³, R¹⁴, R¹⁵, R¹⁶ and R¹⁷ group is independently selected from the group consisting of H, (C₁-C₆)alkyl, —(C═O)N(C₁-C₆)alkyl, —(CR¹⁸R¹⁹)ₚ(C₆-C₁₂)aryl, —(CR¹⁸R¹⁹)ₚ(C₃-C₁₂)cycloalkyl, and —(CR¹⁸R¹⁹)ₚ(4-10)-membered heterocyclyl;
any 1 or 2 carbon atoms of the (4-10)-membered heterocyclyl of each said R⁶, R⁷, R⁸, R⁹, R¹⁰, R¹¹, R¹², R¹³, R¹⁴, R¹⁵, R¹⁶, R¹⁷ group is optionally substituted with an oxo (═O);
any carbon atom of any (C₁-C₆)alkyl, any (C₆-C₁₂)aryl, and any (4-10)-membered heterocyclyl of the foregoing R⁶, R⁷, R⁸, R⁹, R¹⁰, R¹¹, R¹², R¹³, R¹⁴, R¹⁵, R¹⁶, R¹⁷ groups are optionally substituted with 1 to 3 substituents independently selected from the group consisting of halo, cyano, nitro, —NR²⁰R²¹, —CF₃, —CHF₂, —CH₂F, trifluoromethoxy, (C₁-C₆)alkyl, (C₂-C₆)alkenyl, (C₂-C₆)alkynyl, hydroxy, and (C₁-C₆) alkoxy;
each R¹⁸, R¹⁹, R²⁰, and R²¹ group is independently selected from H and (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O or S atom optionally bears on said group a substituent independently selected from the group consisting of hydroxy, halo, (C₁-C₆)alkyl, (C₁-C₆)alkoxy, —NH₂, —NH(C₁-C₆)(alkyl) and —N((C₁-C₆)(alkyl))₂;
or a pharmaceutically acceptable salt or solvate thereof.

2. The compound according to claim 1, wherein b is 2.

3. The compound according to claim 1, wherein T is a 6-membered heterocyclyl containing at least one nitrogen atom.

4. The compound according to claim 1 wherein each R¹ is selected from the group consisting of phenyl, benzothiophenyl, thiazolyl, pyridinyl, piperazinyl, and napthyl, and may optionally be substituted by 1 to 5 R⁵ groups;
wherein:
each R⁵ group is independently selected from the group consisting of halo, cyano, —CF₃, hydroxy, (C₁-C₆)alkoxy, (C₁-C₆)alkyl, (C₂-C₆)alkenyl, —(CR¹⁰R¹¹)ᵥ(4-10)-membered heterocyclyl, —(C═O)—R⁶, —(C═O)—O—R⁶, —O—(C═O)—R⁷, —NR⁸—(C═O)—R⁹, —(C═O)—NR⁸R⁹, —NR⁸R⁹, —NR⁸OR⁹, —(CR¹⁰R¹¹)ᵥ—O—(CR¹⁰R¹¹)ₚ(C₆-C₁₂)aryl, and —(CR¹⁰R¹¹)ₚ—O—(CR¹⁰R¹¹)ₚ(4-10)-membered heterocyclyl.

5. A compound of formula (II): 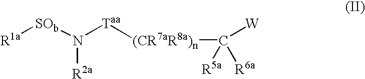
wherein:
R^{1a} is (C₁-C₆)alkyl, —(CR^{7a}R^{8a})ₜ(C₃-C₁₀)cycloalkyl, —(CR^{7a} R^{8a})ₜ(C₆-C₁₀)aryl, or —(CR^{7a}R^{8a})ₜ(4-10)-membered heterocyclyl;
b and k are each independently selected from 1 and 2;
n and j are each independently selected from the group consisting of 0, 1, and 2;
t, u, p, q and v are each independently selected from the group consisting of 0, 1, 2, 3, 4, and 5;
T^{aa} is a (6-10)-membered heterocyclyl containing at least one nitrogen atom;
W is selected from the group consisting of: 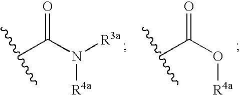
(C₁-C₆) alkyl; and a 5-membered heterocyclyl;
each R^{2a}, R^{3a}, and R^{4a} is independently selected from the group consisting of H, (C₁-C₆)alkyl, (C₂-C₆)alkenyl, (C₂-C₆)alkynyl, —(CR^{7a}R^{8a})ₜ(C₃-C₁₀)cycloalkyl, —(CR^{7a}R^{8a})ₜ(C₆-C₁₀)aryl, and —(CR^{7a}R^{8a})ₜ(4-10)-membered heterocyclyl;
or each R^{3a} and R^{4a} may optionally be taken together with the nitrogen to which they are attached to form a (4-10)-membered heterocyclyl, and the carbon atoms of the (4-10)-membered heterocyclyl may be optionally substituted by 1 to 5 R^{9a} groups;
or each R^{3a} and R^{4a} may optionally be taken together with the nitrogen to which they are attached to form a (4-10)-membered heterocyclyl containing at least one nitrogen atom wherein said at least one nitrogen atom is optionally substituted by at least one substituent selected from the group consisting of —CF₃, —CHF₂, —CH₂F, trifluoromethoxy, (C₁-C₆)alkoxy, (C₁-C₆)alkyl, (C₂-C₆)alkenyl, (C₂-C₆)alkynyl, —(C═O)—R^{7a}, —(C═O)—O—R^{7a}, —(CR^{7a}R^{8a})ᵥ(C₆-C₁₂)aryl, —(CR^{7a}R^{8a})ᵥ(4-10)-membered heterocyclyl, —(CR^{7a}R^{8a})_{q}—(C═O)(CR^{7a}R^{8a})ᵥ(C₆-C₁₂)aryl, and (CR^{7a}R^{8a})_{q}—(C═O)(CR^{7a}R^{8a})ᵥ(4-10)-membered heterocyclyl;
each R^{5a} and R^{6a} are independently selected from the group consisting of H, (C₁-C₆) alkyl, —(CR^{7a}R^{8a})ₜ(C₃-C₁₀)cycloalkyl, —(CR^{7a}R^{8a})ₜ(C₆-C₁₀)aryl, and —(CR^{7a}R^{8a})ₜ(4-10)-membered heterocyclyl;
or R^{5a} and R^{6a} may optionally be taken together with the carbon to which they are attached to form a (C₃-C₆)cycloalkyl or a (3-7)-membered heterocyclyl;
each R^{7a} and R^{8a} is independently selected from H and (C₁-C₆)alkyl;
the carbon atoms of T¹¹, R^{1a}, R^{2a}, R^{3a}, R^{4a}, R^{5a}, R^{6a}, R^{7a}, R^{8a}, and said W 5-membered heterocyclyl are optionally substituted by 1 to 5 R^{9a} groups;
each R^{9a} group is independently selected from the group consisting of halo, cyano, nitro, —CF₃, —CHF₂, —CH₂F, trifluoromethoxy, azido, hydroxy, (C₁-C₆)alkoxy, (C₁-C₆)alkyl, (C₂-C₆)alkenyl, (C₂-C₆)alkynyl, —(C═O)—R^{10a}, —(C═O)—O—R^{11a}, —O—(C═O)—R^{11a}, —NR^{11a}(C═O)—R^{12a}, —(C═O)—NR^{11a}R^{12a}, —NR^{11a}R^{12a}, —NR^{11a}OR^{12a}, —S(O)ₖNR^{11a}R^{12a}, —S(O)ⱼ(C₁-C₆)alkyl, —O—SO₂—R^{10a}, —NR^{11a}—S(O)ₖ—R^{12a}, —(CR^{13a}R^{14a})ᵥ(C₆-C₁₀)aryl, —(CR^{13a}R^{14a})ᵥ(4-10)-membered heterocyclyl, —(CR^{13a}R^{14a})_{q}—(C═O)(CR^{13a}R^{14a})ᵥ(C₆-C₁₀)aryl, —(CR^{13a}R^{14a})_{q}—(C═O)(CR^{13a}R^{14a})ᵥ(4-10)-membered heterocyclyl, —(CR^{13a}R^{14a})ᵥO(CR^{13a}R^{14a})_{q}(C₆-C₁₀)aryl, —(CR^{13a}R^{14a})ᵥO(CR^{13a}R^{14a})_{q}(4-10)-membered heterocyclyl, —(CR^{13a}R^{14a})_{q}S(O)ⱼ(CR^{13a}R^{14a})ᵥ(C₆-C₁₀)aryl, and —(CR^{13a}R^{14a})_{q}S(O)ⱼ(CR^{13a}R^{14a})ᵥ(4-10)-membered heterocyclyl;
any 1 or 2 carbon atoms of any (4-10)-membered heterocyclyl of the foregoing R^{9a} groups are optionally substituted with an oxo (═O);
any carbon atom of any (C₁-C₆)alkyl, any (C₆-C₁₀)aryl and any (4-10)-membered heterocyclyl of the foregoing R^{9a} groups are optionally substituted with 1 to 3 substituents independently selected from the group consisting of halo, cyano, nitro, —CF₃, —CFH₂, —CF₂H, trifluoromethoxy, azido, —OR^{15a}, —(C═O)—R^{15a}, —(C═O)—O—R^{15a}, —O—(C═O)—R^{15a}, —NR^{15a}—(C═O)—R^{16a}, —(C═O)—NR^{15a}R^{16a}, —NR^{15a}R^{16a}, —NR^{15a}O—R^{16a}, (C₁-C₆)alkyl, (C₂-C₆)alkenyl, (C₂-C₆)alkynyl, —(CR^{17a}R^{18a})ᵤ(C₆-C₁₀)aryl, and —(CR^{17a}R^{18a})ᵤ(4-10)-membered heterocyclyl;
each R^{10a}, R^{11a}, R^{12a}, R^{13a}, R^{14a}, R^{15a}, R^{16a}, R^{17a}, and R^{18a} group is independently selected from the group consisting of H, (C₁-C₆)alkyl, —(CR^{19a}R^{20a})ₚ(C₆-C₁₀)aryl, and —(CR^{19a}R^{20a})ₚ(4-10)-membered heterocyclyl;
any 1 or 2 carbon atoms of the (4-10)-membered heterocyclyl of said each R^{10a}, R^{11a}, R^{12a}, R^{13a}, R^{14a}, R^{15a}, R^{16a}, R^{17a}, and R^{18a} group is optionally substituted with an oxo (═O);
any carbon atom of any (C₁-C₆)alkyl, any (C₆-C₁₀)aryl and any (4-10)-membered heterocyclyl of the foregoing R^{10a}, R^{11a}, R^{12a}, R^{13a}, R^{14a}, R^{15a}, R^{16a}, R^{17a}, and R^{18a} groups are optionally substituted with 1 to 3 substituents independently selected from the group consisting of halo, cyano, nitro, —NR^{21a}R^{22a}, —CF₃, —CHF₂, —CH₂F, trifluoromethoxy, (C₁-C₆)alkyl, (C₂-C₆)alkenyl, (C₂-C₆)alkynyl, hydroxy, and (C₁-C₆) alkoxy;
each R^{19a}, R^{20a}, R^{21a}, and R^{22a} group is independently selected from H and (C₁-C₆)alkyl;
and wherein any of the above-mentioned substituents comprising a —CH₃ (methyl), —CH₂ (methylene), or —CH (methine) group which is not attached to a halo, —SO or —SO₂ group or to a N, O, or S atom optionally bears on said group a substituent independently selected from the group consisting of hydroxy, halo, (C₁-C₆)alkyl, (C₁-C₆)alkoxy, amino, —NH(C₁-C₆)(alkyl) and —N((C₁-C₆)(alkyl))₂;
or a pharmaceutically acceptable salt or solvate thereof.

6. The compound according to claim 5, wherein W is

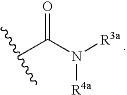

7. The compound according to claim 5, wherein W is

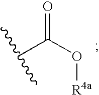

8. The compound according to claim 5, wherein W is a 5-membered heterocyclyl.

9. The compound according to claim 8, wherein said 5-membered heterocyclyl is selected from the group consisting of oxazolyl, thiazolyl, pyrazolyl, triazolyl, and oxadiazolyl.

10. The compound according to claim 5, wherein b is 2.

11. The compound according to claim 5, wherein T is a 6-membered heterocyclyl containing at least one nitrogen atom.

12. The compound according to claim 6, wherein R^{3a} and R^{4a} are taken together with the nitrogen to which they are attached to form a (4-10)-membered heterocyclyl.

13. A compound selected from the group consisting of: 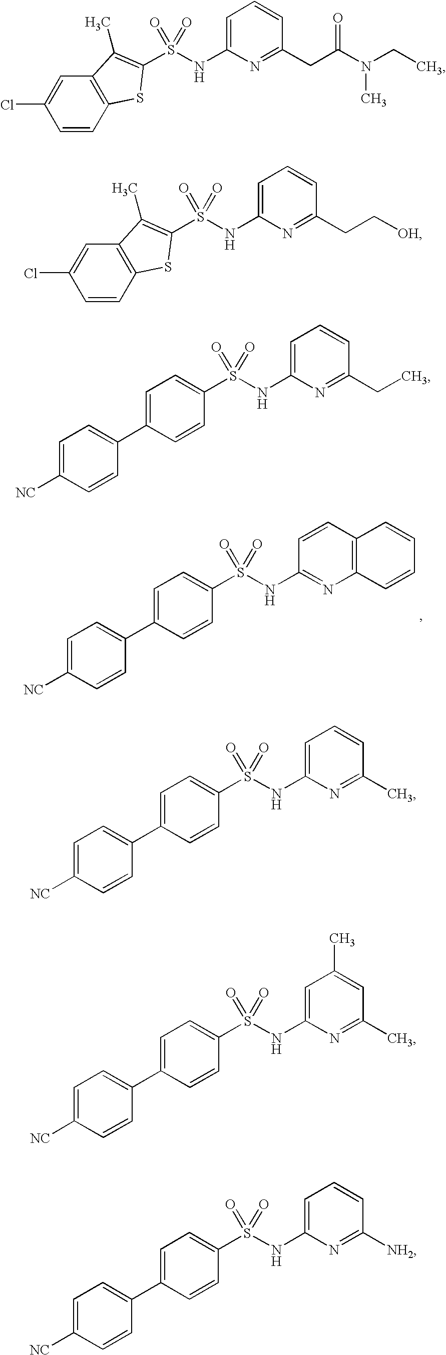 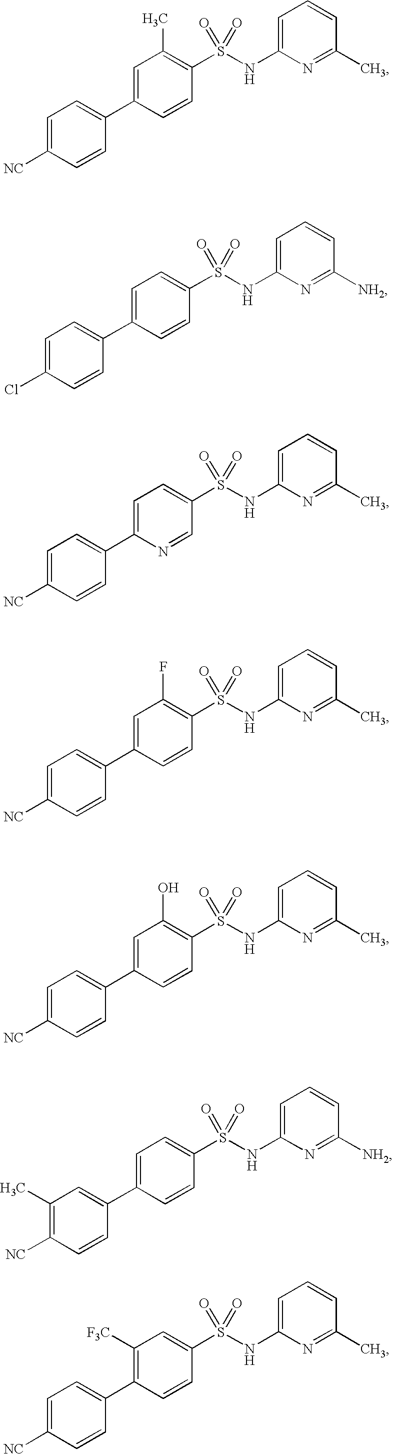 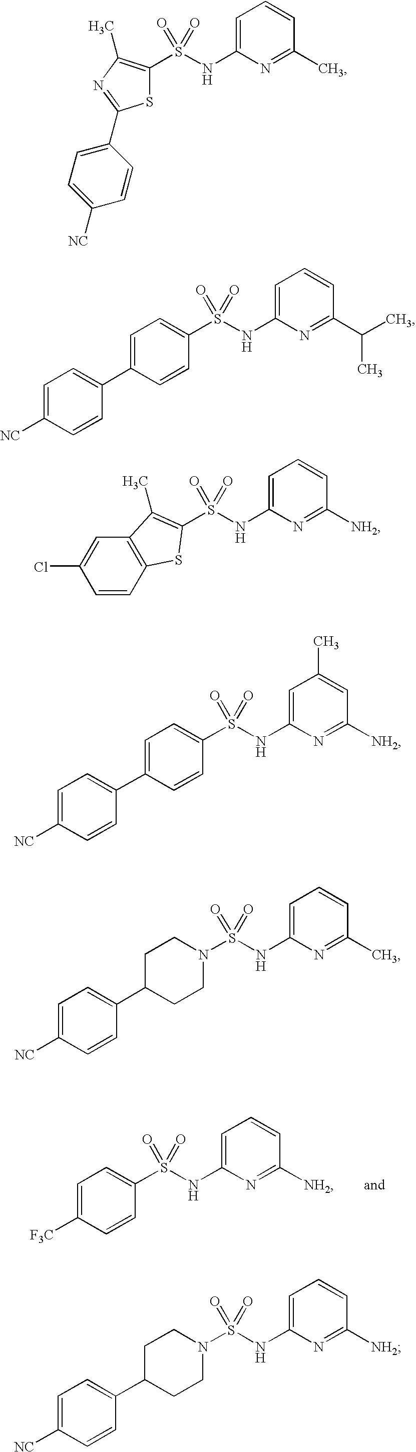
or a pharmaceutically acceptable salt or solvate thereof.

14. A compound of formula (III): 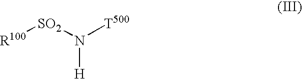
wherein:
R¹⁰⁰ is selected from the group consisting of benzothiophenyl, phenyl, pyridinyl, piperidinyl, and thiazolyl;
the carbon atoms of R¹⁰⁰ may be optionally substituted by 1 to 3 R³⁰⁰ groups;
R³⁰⁰ is selected from the group consisting of hydroxy, (C₁-C₃)alkyl, phenyl, halo, and —CF₃;
the phenyl of the foregoing R³⁰⁰ group may be optionally substituted with 1 to 3 substituents independently selected from the group consisting of halo, cyano, and (C₁-C₃)alkyl;
T⁵⁰⁰ is selected from pyridinyl or quinolinyl;
the carbon atoms of T⁵⁰⁰ may be optionally substituted by 1 to 3 R⁴⁰⁰ groups;
R⁴⁰⁰ is selected from the group consisting of CH₂CH₂—OH, —NH₂, (C₁-C₃)alkyl, and —(C₁-C₃)alkyl-(C═O)—N((C₁-C₃)alkyl)₂;
or a pharmaceutically acceptable salt thereof.

15. A compound according to claim 14, wherein R⁴⁰⁰ is —NH₂.

16. A compound according to claim 14, wherein R⁴⁰⁰ is —CH₃

17. A pharmaceutical composition comprising an effective amount of a compound according to claim 1, or a pharmaceutically acceptable salt or solvate thereof, and a pharmaceutically acceptable carrier.

18. A pharmaceutical composition comprising an effective amount of a compound according to claim 5, or a pharmaceutically acceptable salt or solvate thereof, and a pharmaceutically acceptable carrier.

19. A pharmaceutical composition comprising an effective amount of a compound according to claim 14, or a pharmaceutically acceptable salt or solvate thereof, and a pharmaceutically acceptable carrier.

20. A method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which the liver is a target organ, the method comprising administering to a mammal an effective amount of a compound according to claim 1, or a pharmaceutically acceptable salt or solvate thereof.

21. A method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which the liver is a target organ, the method comprising administering to a mammal an effective amount of a compound according to claim 5, or a pharmaceutically acceptable salt or solvate thereof.

22. A method of treating diabetes, metabolic syndrome, insulin resistance syndrome, obesity, glaucoma, hyperlipidemia, hyperglycemia, hyperinsulinemia, osteoporosis, tuberculosis, atherosclerosis, dementia, depression, virus diseases, inflammatory disorders, or diseases in which the liver is a target organ, the method comprising administering to a mammal an effective amount of a compound according to claim 14, or a pharmaceutically acceptable salt or solvate thereof.
